(19) United States
(12) Patent Application Publication
(10) Pub. No.: US 2007/0092917 A1
(43) Pub. Date: Apr. 26, 2007

(54) Biomarkers for screening, predicting, and monitoring prostate disease

(76) Inventor: Isabelle Guyon, Berkeley, CA (US)

Correspondence Address: PROCOPIO, CORY, HARGREAVES & SAVITCH LLP, 530 B STREET, SUITE 2100, SAN DIEGO, CA, 92101, US

(21) Appl. No.: 11/274,931

(22) Filed: Nov. 14, 2005

Related U.S. Application Data
(63) Continuation-in-part of application No. 10/057,849, filed on Jan. 24, 2002, now Pat. No. 7,117,188.
(63) Continuation-in-part of application No. 09/633,410, filed on Aug. 7, 2000, now Pat. No. 6,882,990.
(63) Continuation-in-part of application No. 09/578,011, filed on May 24, 2000, now Pat. No. 6,658,395.
(63) Continuation-in-part of application No. 09/568,301, filed on May 9, 2000, now Pat. No. 6,427,141.
Application No. 09/568,301 is a continuation of application No. 09/303,387, filed on May 1, 1999, now Pat. No. 6,128,608.
(60) Provisional application No. 60/627,626, filed on Nov. 12, 2004.
(60) Provisional application No. 60/651,340, filed on Feb. 9, 2005.
(60) Provisional application No. 60/263,696, filed on Jan. 24, 2001.
(60) Provisional application No. 60/298,757, filed on Jun. 15, 2001.
(60) Provisional application No. 60/275,760, filed on Mar. 14, 2001.
(60) Provisional application No. 60/161,806, filed on Oct. 27, 1999.
(60) Provisional application No. 60/168,703, filed on Dec. 2, 1999.
(60) Provisional application No. 60/207,026, filed on May 25, 2000.
(60) Provisional application No. 60/191,219, filed on Mar. 22, 2000.
(60) Provisional application No. 60/184,596, filed on Feb. 24, 2000.
(60) Provisional application No. 60/135,715, filed on May 25, 1999.
(60) Provisional application No. 60/083,961, filed on May 1, 1998.

(51) Int. Cl. G01N 33/574

(52) U.S. Cl. 435/7.23

(57) ABSTRACT

Gene expression data are analyzed using learning machines such as support vector machines (SVM) and ridge regression classifiers to rank genes according to their ability to separate prostate cancer from BPH (benign prostatic hyperplasia) and to distinguish cancer volume. Other tests identify biomarker candidates for distinguishing between tumor (Grade 3 and Grade 4 (G3/4)) and normal tissue.

### RELATED APPLICATIONS

The present application claims priority to each of U.S. Provisional Applications No. 60/627,626, filed Nov. 12, 2004, and No. 60/651,340, filed Feb. 9, 2005, and is a continuation-in-part of U.S. application Ser. No. 10/057/849, which claims priority to each of U.S. Provisional Applications No. 60/263,696, filed Jan. 24, 2001, No. 60/298,757, filed Jun. 15, 2001, and No. 60/275,760, filed Mar. 14, 2001, and is a continuation-in-part of U.S. patent application Ser. No. 09/633,410, filed Aug. 7, 2000, now issued as U.S. Pat. No. 6,882,990, which claims priority to each of U.S. Provisional Applications No. 60/161,806, filed Oct. 27, 1999, No. 60/168,703, filed Dec. 2, 1999, No. 60/184,596, filed Feb. 24, 2000, No. 60/191,219, filed Mar. 22, 2000, and No. 60/207,026, filed May 25, 2000, and is a continuation-in-part of U.S. patent application Ser. No. 09/578,011, filed May 24, 2000, now issued as U.S. Pat. No. 6,658,395, which claims priority to U.S. Provisional Application No. 60/135,715, filed May 25, 1999, and is a continuation-in-part of application Ser. No. 09/568,301, filed May 9, 2000, now issued as U.S. Pat. No. 6,427,141, which is a continuation of application Ser. No. 09/303,387, filed May 1, 1999, now issued as U.S. Pat. No. 6,128,608, which claims priority to U.S. Provisional Application No. 60/083,961, filed May 1, 1998. This application is related to co-pending application Ser. No. 09/633,615, now abandoned, Ser. No. 09/633,616, now issued as U.S. Pat. No. 6,760,715, Ser. No. 09/633,627, now issued as U.S. Pat. No. 6,714,925, and Ser. No. 09/633,850, now issued as U.S. Pat. No. 6,789,069, all filed Aug. 7, 2000, which are also continuations-in-part of application Ser. No. 09/578,011. Each of the above cited applications and patents are incorporated herein by reference.

### FIELD OF THE INVENTION

The present invention relates to the use of learning machines to identify relevant patterns in datasets containing large quantities of gene expression data, and more particularly to biomarkers so identified for use in screening, predicting, and monitoring prostate cancer.

### BACKGROUND OF THE INVENTION

Enormous amounts of data about organisms are being generated in the sequencing of genomes. Using this information to provide treatments and therapies for individuals will require an in-depth understanding of the gathered information. Efforts using genomic information have already led to the development of gene expression investigational devices. One of the most currently promising devices is the gene chip. Gene chips have arrays of oligonucleotide probes attached a solid base structure. Such devices are described in U.S. Pat. Nos. 5,837,832 and 5,143,854, herein incorporated by reference in their entirety. The oligonucleotide probes present on the chip can be used to determine whether a target nucleic acid has a nucleotide sequence identical to or different from a specific reference sequence. The array of probes comprise probes that are complementary to the reference sequence as well as probes that differ by one of more bases from the complementary probes.

The gene chips are capable of containing large arrays of oliogonucleotides on very small chips. A variety of methods for measuring hybridization intensity data to determine which probes are hybridizing is known in the art. Methods for detecting hybridization include fluorescent, radioactive, enzymatic, chemoluminescent, bioluminescent and other detection systems.

Older, but still usable, methods such as gel electrophosesis and hybridization to gel blots or dot blots are also useful for determining genetic sequence information. Capture and detection systems for solution hybridization and in situ hybridization methods are also used for determining information about a genome. Additionally, former and currently used methods for defining large parts of genomic sequences, such as chromosome walking and phage library establishment, are used to gain knowledge about genomes.

Large amounts of information regarding the sequence, regulation, activation, binding sites and internal coding signals can be generated by the methods known in the art. In fact, the voluminous amount of data being generated by such methods hinders the derivation of useful information. Human researchers, when aided by advanced learning tools such as neural networks can only derive crude models of the underlying processes represented in the large, feature-rich datasets.

In recent years, technologies have been developed that can relate gene expression to protein production structure and function. Automated high-throughput analysis, nucleic acid analysis and bioinformatics technologies have aided in the ability to probe genomes and to link gene mutations and expression with disease predisposition and progression. The current analytical methods are limited in their abilities to manage the large amounts of data generated by these technologies.

Machine-learning approaches for data analysis have been widely explored for recognizing patterns which, in turn, allow extraction of significant information contained within a large data set which may also include data that provide nothing more than irrelevant detail. Learning machines comprise algorithms that may be trained to generalize using data with known outcomes. Trained learning machine algorithms may then be applied to predict the outcome in cases of unknown outcome. Machine-learning approaches, which include neural networks, hidden Markov models, belief networks, and support vector machines, are ideally suited for domains characterized by the existence of large amounts of data, noisy patterns, and the absence of general theories.

Support vector machines were introduced in 1992 and the “kernel trick” was described. See Boser, B, et al., in Fifth Annal Workship on Computational Learning Theory, p 144-152, Pittsburgh, ACM which is herein incorporated in its entirety. A training algorithm that maximizes the margin between the training patterns and the decision boundary was presented. The techniques was applicable to a wide variety of classification functions, including Perceptrons, polynomials, and Radial Basis Functions. The effective number of parameters was adjusted automaticaly to match the complexity of the problem. The solution was expressed as a linear combination of supporting patterns. These are the subset of training patterns that are closest to the decision boundary. Bounds on the generalization performance based on the leave-one-out method and the VC-dimension are given. Experimental results on optical character recognition problems demonstrate the good generalization obtained when compared with other learning algorithms.

Once patterns or the relationships between the data are identified by the support vector machines and are used to detect or diagnose a particular disease state, diagnostic tests, including gene chips and tests of bodily fluids or bodily changes, and methods and compositions for treating the condition, and for monitoring the effectiveness of the treatment, are needed

A significant fraction of men (20%) in the U.S. are diagnosed with prostate cancer during their lifetime, with nearly 300,000 men diagnosed annually, a rate second only to skin cancer. However, only 3% of those die from the disease. About 70% of all diagnosed prostate cancers are found in men aged 65 years and older. Many prostate cancer patients have undergone aggressive treatments that can have life-altering side effects such as incontinence and sexual dysfunction. It is believed that a large fraction of the cancers are over-treated. Currently, most early prostate cancer identification is done using prostate-specific antigen (PSA) screening, but few indicators currently distinguish between progressive prostate tumors that may metastasize and escape local treatment and indolent cancers of benign prostate hyperplasia (BPH). Further, some studies have shown that PSA is a poor predictor of cancer, instead tending to predict BPH, which requires no treatment.

The development of diagnosis assays in a rapidly changing technology environment is challenging. Collecting samples and processing them with genomics or proteomics measurement instruments is costly and time consuming, so the development of a new assay is often done with as little as 100 samples. Statisticians warn of the sad reality of statistical significance, which means that with so few samples, biomarker discovery is very unreliable. Furthermore, no accurate prediction of diagnosis accuracy can be made. There is an urgent need for new biomarkers for distinguishing between normal, benign, and malignant prostate tissue and for predicting the size and malignancy of prostate cancer. Blood serum biomarkers would be particularly desirable for screening prior to biopsy, however, evaluation of gene expression microarrays from biopsied prostate tissue is also useful.

### SUMMARY OF THE INVENTION

Gene expression data are analyzed using learning machines such as support vector machines (SVM) and ridge regression classifiers to rank genes according to their ability to separate prostate cancer from BPH (benign prostatic hyperplasia) and to distinguish cancer volume. Other tests identify biomarker candidates for distinguishing between tumor (Grade 3 and Grade 4 (G3/4)) and normal tissue.

The present invention comprises systems and methods for enhancing knowledge discovered from data using a learning machine in general and a support vector machine in particular. In particular, the present invention comprises methods of using a learning machine for diagnosing and prognosing changes in biological systems such as diseases. Further, once the knowledge discovered from the data is determined, the specific relationships discovered are used to diagnose and prognose diseases, and methods of detecting and treating such diseases are applied to the biological system. In particular, the invention is directed to detection of genes involved with prostate cancer and determining methods and compositions for treatment of prostate cancer.

In a preferred embodiment, the support vector machine is trained using a pre-processed training data set. Each training data point comprises a vector having one or more coordinates. Pre-processing of the training data set may comprise identifying missing or erroneous data points and taking appropriate steps to correct the flawed data or, as appropriate, remove the observation or the entire field from the scope of the problem, i.e., filtering the data. Pre-processing the training data set may also comprise adding dimensionality to each training data point by adding one or more new coordinates to the vector. The new coordinates added to the vector may be derived by applying a transformation to one or more of the original coordinates. The transformation may be based on expert knowledge, or may be computationally derived. In this manner, the additional representations of the training data provided by preprocessing may enhance the learning machine's ability to discover knowledge therefrom. In the particular context of support vector machines, the greater the dimensionality of the training set, the higher the quality of the generalizations that may be derived therefrom.

A test data set is pre-processed in the same manner as was the training data set. Then, the trained learning machine is tested using the pre-processed test data set. A test output of the trained learning machine may be post-processing to determine if the test output is an optimal solution. Post-processing the test output may comprise interpreting the test output into a format that may be compared with the test data set. Alternative postprocessing steps may enhance the human interpretability or suitability for additional processing of the output data.

The process of optimizing the classification ability of a support vector machine includes the selection of at least one kernel prior to training the support vector machine. Selection of a kernel may be based on prior knowledge of the specific problem being addressed or analysis of the properties of any available data to be used with the learning machine and is typically dependant on the nature of the knowledge to be discovered from the data. Optionally, an iterative process comparing postprocessed training outputs or test outputs can be applied to make a determination as to which kernel configuration provides the optimal solution. If the test output is not the optimal solution, the selection of the kernel may be adjusted and the support vector machine may be retrained and retested. When it is determined that the optimal solution has been identified, a live data set may be collected and pre-processed in the same manner as was the training data set. The pre-processed live data set is input into the learning machine for processing. The live output of the learning machine may then be post-processed to generate an alphanumeric classifier or other decision to be used by the researcher or clinician, e.g., yes or no, or, in the case of cancer diagnosis, malignent or benign.

A preferred embodiment comprises methods and systems for detecting genes involved with prostate cancer and determination of methods and compositions for treatment of prostate cancer. In one embodiment, to improve the statistical significance of the results, supervised learning techniques can analyze data obtained from a number of different sources using different microarrays, such as the Affymetrix U95 and U133A chip sets.

### BRIEF DESCRIPTION OF THE DRAWINGS

FIG. 1 is a functional block diagram illustrating an exemplary operating environment for an embodiment of the present invention.

FIG. 2 is a functional block diagram illustrating a hierarchical system of multiple support vector machines.

FIG. 3 illustrates a binary tree generated using an exemplary SVM-RFE.

FIGS. 4a-4d illustrate an observation graph used to generate the binary tree of FIG. 3, where FIG. 4a shows the oldest descendents of the root labeled by the genes obtained from regular SVM-RFE gene ranking; FIG. 4b shows the second level of the tree filled with top ranking genes from root to leaf after the top ranking gene of FIG. 4a is removed, and SVM-RFE is run again; FIG. 4c shows the second child of the oldest node of the root and its oldest descendents labeled by using constrained RFE; and FIG. 4d shows the first and second levels of the tree filled root to leaf and the second child of each root node filled after the top ranking genes in FIG. 4c are removed.

FIG. 5 is a plot showing the results based on LCM data preparation for prostate cancer analysis.

FIG. 6 is a plot graphically comparing SVM-RFE of the present invention with leave-one-out classifier for prostate cancer.

FIG. 7 graphically compares the Golub and SVM methods for prostate cancer.

FIGS. 8a and 8b combined are a table showing the ranking of the top 50 genes using combined criteria for selecting genes according to disease severity.

FIGS. 9a and 9b combined are a table showing the ranking of the top 50 genes for disease progression obtained using Pearson correlation criterion.

FIGS. 10a-10e combined are a table showing the ranking of the top 200 genes separating BPH from other tissues.

FIG. 11a-11e combined are a table showing the ranking of the top 200 genes for separating prostate tumor from other tissues.

FIG. 12a-12e combined are a table showing the top 200 genes for separating G4 tumor from other tissues.

FIG. 13a-c combined are a table showing the top 100 genes separating normal prostate from all other tissues.

FIG. 14 is a table listing the top 10 genes separating G3 tumor from all other tissues.

FIG. 15 is a table listing the top 10 genes separating Dysplasia from all other tissues.

FIG. 16 is a table listing the top 10 genes separating G3 prostate tumor from G3 tumor.

FIG. 17 is a table listing the top 10 genes separating normal tissue from Dysplasia.

FIG. 18 is a table listing the top 10 genes for separating transition zone G4 from peripheral zone G4 tumor.

FIG. 19 is a table listing the top 9 genes most correlated with cancer volume in G3 and G4 samples.

FIG. 20a-20o combined are two tables showing the top 200 genes for separating G3 and G4 tumor from all others for each of the 2001 study and the 2003 study.

FIG. 21 is a scatter plot showing the correlation between the 2001 study and the 2003 study for tumor versus normal.

FIG. 22 is a plot showing reciprocal feature set enrichment for the 2001 study and the 2003 study for separating tumor from normal.

FIG. 23a-23g combined are a table showing the top 200 genes for separating G3 and G4 tumor versus others using feature ranking by consensus between the 2001 study and the 2003 study.

FIG. 24a-24s combined are two tables showing the top 200 genes for separating BPH from all other tissues that were identified in each of the 2001 study and the 2003 study.

FIG. 25a-25h combined are a table showing the top 200 genes for separating BPH from all other tissues using feature ranking by consensus between the 2001 study and the 2003 study.

FIG. 26a-26bb combined are a table showing the top 200 genes for separating G3 and G4 tumors from all others that were identified in each of the public data sets and the 2003 study.

FIG. 27a-27l combined are a table showing the top 200 genes for separating tumor from normal using feature ranking by consensus between the public data and the 2003 study.

FIG. 28 is a diagram of a hierarchical decision tree for BPH, G3 & G4, Dysplasia, and Normal cells.

### DETAILED DESCRIPTION OF THE PREFERRED EMBODIMENT

The present invention utilizes learning machine techniques, including support vector machines and ridge regression, to discover knowledge from gene expression data obtained by measuring hybridization intensity of gene and gene fragment probes on microarrays. The knowledge so discovery can be used for diagnosing and prognosing changes in biological systems, such as diseases. Preferred embodiments comprise identification of genes involved with prostate disorders including benign prostate hyperplasy and cancer and use of such information for decisions on treatment of patients with prostate disorders.

The problem of selection of a small amount of data from a large data source, such as a gene subset from a microarray, is particularly solved using the methods described herein. Preferred methods described herein use support vector machines methods based on recursive feature elimination (RFE). In examining genetic data to find determinative genes, these methods eliminate gene redundancy automatically and yield better and more compact gene subsets.

According to the preferred embodiment, gene expression data is pre-processed prior to using the data to train a learning machine. Generally stated, pre-processing data comprises reformatting or augmenting the data in order to allow the learning machine to be applied most advantageously. In a manner similar to pre-processing, post-processing involves interpreting the output of a learning machine in order to discover meaningful characteristics thereof. The meaningful characteristics to be ascertained from the output may be problem- or data-specific. Post-processing involves interpreting the output into a form that, for example, may be understood by or is otherwise useful to a human observer, or converting the output into a form which may be readily received by another device for, e.g., archival or transmission.

There are many different methods for analyzing large data sources. Errorless separation can be achieved with any number of genes greater than one. Preferred methods comprise use of a smaller number of genes. Classical gene selection methods select the genes that individually best classify the training data. These methods include correlation methods and expression ratio methods. While the classical methods eliminate genes that are useless for discrimination (noise), they do not yield compact gene sets because genes are redundant. Moreover, complementary genes that individually do not separate well are missed.

A simple feature (gene) ranking can be produced by evaluating how well an individual feature contributes to the separation (e.g. cancer vs. normal). Various correlation coefficients have been used as ranking criteria. See, e.g., T. K. Golub, et al, “Molecular classification of cancer: Class discovery and class prediction by gene expression monitoring”, Science 286, 531-37 (1999), incorporated herein by reference. The method described by Golub, et al. for feature ranking is to select an equal number of genes with positive and with negative correlation coefficients. Each coefficient is computed with information about a single feature (gene) and, therefore, does not take into account mutual information between features.

One use of feature ranking is in the design of a class predictor (or classifier) based on a pre-selected subset of genes. Each feature that is correlated (or anti-correlated) with the separation of interest is by itself such a class predictor, albeit an imperfect one. A simple method of classification comprises a method based on weighted voting: the features vote in proportion to their correlation coefficient. Such is the method used by Golub, et al.

Another classifier or class predictor is Fisher's linear discriminant, which is similar to that of Golub et al. This method yields an approximation that may be valid if the features are uncorrelated, however, features in gene expression data usually are correlated and, therefore, such an approximation is not valid.

The present invention uses the feature ranking coefficients as classifier weights. Reciprocally, the weights multiplying the inputs of a given classifier can be used as feature ranking coefficients. The inputs that are weighted by the largest values have the most influence in the classification decision. Therefore, if the classifier performs well, those inputs with largest weights correspond to the most informative features, or in this instance, genes. Other methods, known as multivariate classifiers, comprise algorithms to train linear discriminant functions that provide superior feature ranking compared to correlation coefficients. Multivariate classifiers, such as the Fisher's linear discriminant (a combination of multiple univariate classifiers) and methods disclosed herein, are optimized during training to handle multiple variables or features simultaneously.

For classification problems, the ideal objective function is the expected value of the error, i.e., the error rate computed on an infinite number of examples. For training purposes, this ideal objective is replaced by a cost function J computed on training examples only. Such a cost function is usually a bound or an approximation of the ideal objective, selected for convenience and efficiency. For linear SVMs, the cost function is:
Error! Objects cannot be created from editing field codes., (1)
which is minimized, under constraints, during training. The criteria (wᵢ)² estimates the effect on the objective (cost) function of removing feature i.

A good feature ranking criterion is not necessarily a good criterion for ranking feature subsets. Some criteria estimate the effect on the objective function of removing one feature at a time. These criteria become suboptimal when several features are removed at one time, which is necessary to obtain a small feature subset. Recursive Feature Elimination (RFE) methods can be used to overcome this problem. RFE methods comprise iteratively 1) training the classifier, 2) computing the ranking criterion for all features, and 3) removing the feature having the smallest ranking criterion. This iterative procedure is an example of backward feature elimination. For computational reasons, it may be more efficient to remove several features at a time at the expense of possible classification performance degradation. In such a case, the method produces a “feature subset ranking”, as opposed to a “feature ranking”. Feature subsets are nested, e.g., F₁⊂F₂⊂ . . . ⊂F.

If features are removed one at a time, this results in a corresponding feature ranking. However, the features that are top ranked, i.e., eliminated last, are not necessarily the ones that are individually most relevant. It may be the case that the features of a subset Fₘ are optimal in some sense only when taken in some combination. RFE has no effect on correlation methods since the ranking criterion is computed using information about a single feature.

In general, RFE can be computationally expensive when compared against correlation methods, where several thousands of input data points can be ranked in about one second using a Pentium® processor, and weights of the classifier trained only once with all features, such as SVMs or pseudo-inverse/mean squared error (MSE). A SVM implemented using non-optimized MatLab® code on a Pentium® processor can provide a solution in a few seconds. To increase computational speed, RFE is preferrably implemented by training multiple classifiers on subsets of features of decreasing size. Training time scales linearly with the number of classifiers to be trained. The trade-off is computational time versus accuracy. Use of RFE provides better feature selection than can be obtained by using the weights of a single classifier. Better results are also obtained by eliminating one feature at a time as opposed to eliminating chunks of features. However, significant differences are seen only for a smaller subset of features such as fewer than 100. Without trading accuracy for speed, RFE can be used by removing chunks of features in the first few iterations and then, in later iterations, removing one feature at a time once the feature set reaches a few hundreds. RFE can be used when the number of features, e.g., genes, is increased to millions. Furthermore, RFE consistently outperforms the naïve ranking, particularly for small feature subsets. (The naïve ranking comprises ranking the features with (wᵢ)², which is computationally equivalent to the first iteration of RFE.) The naïve ranking orders features according to their individual relevance, while RFE ranking is a feature subset ranking. The nested feature subsets contain complementary features that individually are not necessarily the most relevant. An important aspect of SVM feature selection is that clean data is most preferred because outliers play an essential role. The selection of useful patterns, support vectors, and selection of useful features are connected.

The data is input into computer system, preferably a SVM-RFE. The SVM-RFE is run one or more times to generate the best features selections, which can be displayed in an observation graph. The SVM may use any algorithm and the data may be preprocessed and postprocessed if needed. Preferably, a server contains a first observation graph that organizes the results of the SVM activity and selection of features.

The information generated by the SVM may be examined by outside experts, computer databases, or other complementary information sources. For example, if the resulting feature selection information is about selected genes, biologists or experts or computer databases may provide complementary information about the selected genes, for example, from medical and scientific literature. Using all the data available, the genes are given objective or subjective grades. Gene interactions may also be recorded.

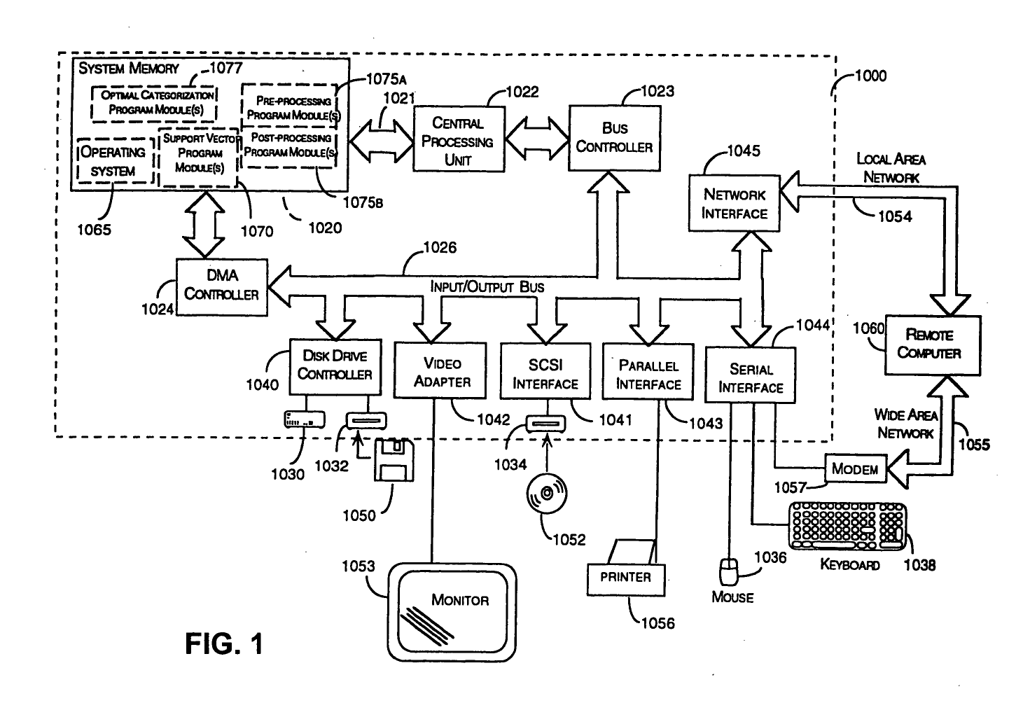

FIG. 1 and the following discussion are intended to provide a brief and general description of a suitable computing environment for implementing biological data analysis according to the present invention. Although the system shown in FIG. 1 is a conventional personal computer 1000, those skilled in the art will recognize that the invention also may be implemented using other types of computer system configurations. The computer 1000 includes a central processing unit 1022, a system memory 1020, and an Input/Output (“I/O”) bus 1026. A system bus 1021 couples the central processing unit 1022 to the system memory 1020. A bus controller 1023 controls the flow of data on the I/O bus 1026 and between the central processing unit 1022 and a variety of internal and external I/O devices. The I/O devices connected to the I/O bus 1026 may have direct access to the system memory 1020 using a Direct Memory Access (“DMA”) controller 1024.

The I/O devices are connected to the I/O bus 1026 via a set of device interfaces. The device interfaces may include both hardware components and software components. For instance, a hard disk drive 1030 and a floppy disk drive 1032 for reading or writing removable media 1050 may be connected to the I/O bus 1026 through disk drive controllers 1040. An optical disk drive 1034 for reading or writing optical media 1052 may be connected to the I/O bus 1026 using a Small Computer System Interface (“SCSI”) 1041. Alternatively, an IDE (Integrated Drive Electronics, i.e., a hard disk drive interface for PCs), ATAPI (ATtAchment Packet Interface, i.e., CD-ROM and tape drive interface), or EIDE (Enhanced IDE) interface may be associated with an optical drive such as may be the case with a CD-ROM drive. The drives and their associated computer-readable media provide nonvolatile storage for the computer 1000. In addition to the computer-readable media described above, other types of computer-readable media may also be used, such as ZIP drives, or the like.

A display device 1053, such as a monitor, is connected to the I/O bus 1026 via another interface, such as a video adapter 1042. A parallel interface 1043 connects synchronous peripheral devices, such as a laser printer 1056, to the I/O bus 1026. A serial interface 1044 connects communication devices to the I/O bus 1026. A user may enter commands and information into the computer 1000 via the serial interface 1044 or by using an input device, such as a keyboard 1038, a mouse 1036 or a modem 1057. Other peripheral devices (not shown) may also be connected to the computer 1000, such as audio input/output devices or image capture devices.

A number of program modules may be stored on the drives and in the system memory 1020. The system memory 1020 can include both Random Access Memory (“RAM”) and Read Only Memory (“ROM”). The program modules control how the computer 1000 functions and interacts with the user, with I/O devices or with other computers. Program modules include routines, operating systems 1065, application programs, data structures, and other software or firmware components. In an illustrative embodiment, the learning machine may comprise one or more pre-processing program modules 1075A, one or more post-processing program modules 1075B, and/or one or more optimal categorization program modules 1077 and one or more SVM program modules 1070 stored on the drives or in the system memory 1020 of the computer 1000. Specifically, pre-processing program modules 1075A, post-processing program modules 1075B, together with the SVM program modules 1070 may comprise computer-executable instructions for pre-processing data and post-processing output from a learning machine and implementing the learning algorithm. Furthermore, optimal categorization program modules 1077 may comprise computer-executable instructions for optimally categorizing a data set.

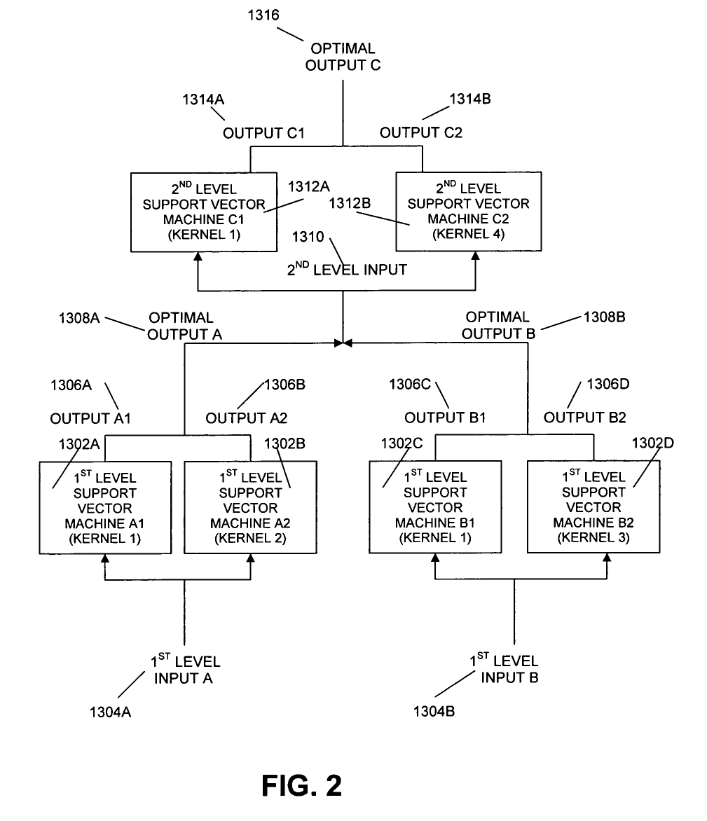

The computer 1000 may operate in a networked environment using logical connections to one or more remote computers, such as remote computer 1060. The remote computer 1060 may be a server, a router, a peer to peer device or other common network node, and typically includes many or all of the elements described in connection with the computer 1000. In a networked environment, program modules and data may be stored on the remote computer 1060. The logical connections depicted in FIG. 2 include a local area network (“LAN”) 1054 and a wide area network (“WAN”) 1055. In a LAN environment, a network interface 1045, such as an Ethernet adapter card, can be used to connect the computer 1000 to the remote computer 1060. In a WAN environment, the computer 1000 may use a telecommunications device, such as a modem 1057, to establish a connection. It will be appreciated that the network connections shown are illustrative and other devices of establishing a communications link between the computers may be used.

A preferred selection browser is preferably a graphical user interface that would assist final users in using the generated information. For example, in the examples herein, the selection browser is a gene selection browser that assists the final user is selection of potential drug targets from the genes identified by the SVM RFE. The inputs are the observation graph, which is an output of a statistical analysis package and any complementary knowledge base information, preferably in a graph or ranked form. For example, such complementary information for gene selection may include knowledge about the genes, functions, derived proteins, measurement assays, isolation techniques, etc. The user interface preferably allows for visual exploration of the graphs and the product of the two graphs to identify promising targets. The browser does not generally require intensive computations and if needed, can be run on other computer means. The graph generated by the server can be precomputed, prior to access by the browser, or is generated in situ and functions by expanding the graph at points of interest.

In a preferred embodiment, the server is a statistical analysis package, and in the gene feature selection, a gene selection server. For example, inputs are patterns of gene expression, from sources such as DNA microarrays or other data sources. Outputs are an observation graph that organizes the results of one or more runs of SVM RFE. It is optimum to have the selection server run the computationally expensive operations.

A preferred method of the server is to expand the information acquired by the SVM. The server can use any SVM results, and is not limited to SVM RFE selection methods. As an example, the method is directed to gene selection, though any data can be treated by the server. Using SVM RFE for gene selection, gene redundancy is eliminated, but it is informative to know about discriminant genes that are correlated with the genes selected. For a given number N of genes, only one combination is retained by SVM-RFE. In actuality, there are many combinations of N different genes that provide similar results.

A combinatorial search is a method allowing selection of many alternative combinations of N genes, but this method is prone to overfitting the data. SVM-RFE does not overfit the data. SVM-RFE is combined with supervised clustering to provide lists of alternative genes that are correlated with the optimum selected genes. Mere substitution of one gene by another correlated gene yields substantial classification performance degradation.

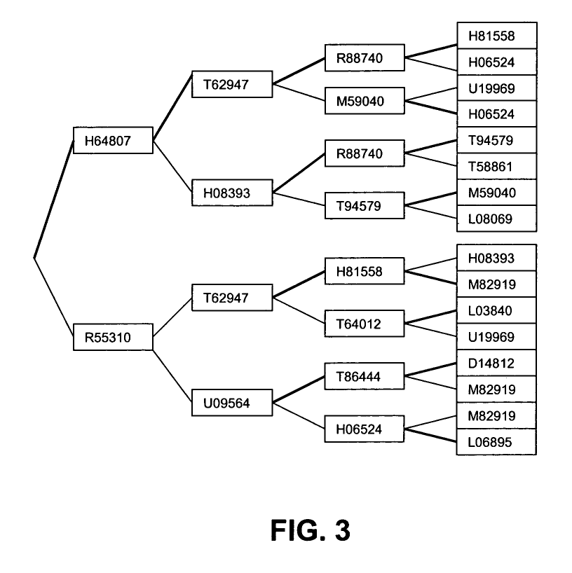

An example of an observation graph containing several runs of SVM-RFE for colon data is shown in FIG. 3. A path from the root node to a given node in the tree at depth D defines a subset of D genes. The quality of every subset of genes can be assessed, for example, by the success rate of a classifier trained with these genes.

The graph has multiple uses. For example, in designing a therapeutic composition that uses a maximum of four proteins, the statistical analysis does not take into account which proteins are easier to provide to a patient. In the graph, the preferred unconstrained path in the tree is indicated by the bold edges in the tree, from the root node to the darkest leaf node. This path corresponds to running a SVM-RFE. If it is found that the gene selected at this node is difficult to use, a choice can be made to use the alternative protein, and follow the remaining unconstrained path, indicated by bold edges. This decision process can be optimized by using the notion of search discussed below in a product graph.

In FIG. 3, a binary tree of depth 4 is shown. This means that for every gene selection, there are only two alternatives and selection is limited to four genes. Wider trees allow for selection from a wider variety of genes. Deeper trees allow for selection of a larger number of genes.

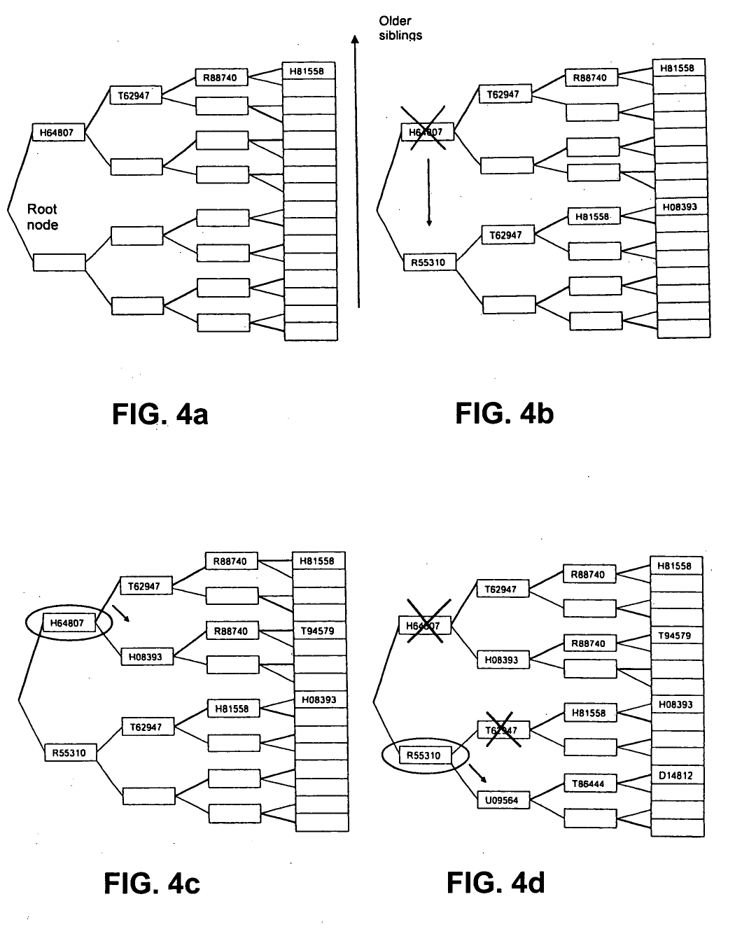

An example of construction of the tree of the observation graph is presented herein and shown in FIGS. 4a-d, which show the steps of the construction of the tree of FIG. 3. In FIG. 4a, all of the oldest descendents of the root are labeled by the genes obtained from regular SVM-RFE gene ranking. The best ranking gene is closest to the root node. The other children of the root, from older to younger, and all their oldest decendents are then labeled. In the case of a binary tree, there are only two branches, or children, of any one node (4b). The top ranking gene of FIG. 4a is removed, and SVM-RFE is run again. This second level of the tree is filled with the top ranking genes, from root to leaf. At this stage, all the nodes that are at depth 1 are labeled with one gene. In moving to fill the second level, the SVM is run using constrained RFE. The constraint is that the gene of the oldest node must never be eliminated. The second child of the oldest node of root and all its oldest descendents are labeled by running the constrained RFE.

The examples included herein show preferred methods for determining the genes that are most correlated to the presence of cancer or can be used to predict cancer occurrence in an individual. There is no limitation to the source of the data and the data can be combinations of measurable criteria, such as genes, proteins or clinical tests, that are capable of being used to differentiate between normal conditions and changes in conditions in biological systems.

In the following examples, preferred numbers of genes were determined that result from separation of the data that discriminate. These numbers are not limiting to the methods of the present invention. Preferably, the preferred optimum number of genes is a range of approximately from 1 to 500, more preferably, the range is from 10 to 250, from 1 to 50, even more preferably the range is from 1 to 32, still more preferably the range is from 1 to 21 and most preferably, from 1 to 10. The preferred optimum number of genes can be affected by the quality and quantity of the original data and thus can be determined for each application by those skilled in the art.

Once the determinative genes are found by the learning machines of the present invention, methods and compositions for treatments of the biological changes in the organisms can be employed. For example, for the treatment of cancer, therapeutic agents can be administered to antagonize or agonize, enhance or inhibit activities, presence, or synthesis of the gene products. Therapeutic agents and methods include, but are not limited to, gene therapies such as sense or antisense polynucleotides, DNA or RNA analogs, pharmaceutical agents, plasmaphoresis, antiangiogenics, and derivatives, analogs and metabolic products of such agents.

Such agents may be administered via parenteral or noninvasive routes. Many active agents are administered through parenteral routes of administration, intravenous, intramuscular, subcutaneous, intraperitoneal, intraspinal, intrathecal, intracerebroventricular, intraarterial and other routes of injection. Noninvasive routes for drug delivery include oral, nasal, pulmonary, rectal, buccal, vaginal, transdermal and ocular routes.

The following examples illustrate the use of SVMs and other learning machines for the purpose of identifying genes associated with disorders of the prostate. Such genes may be used for diagnosis, treatment, in terms of identifying appropriate therapeutic agents, and for monitoring the progress of treatment.

### EXAMPLE 1

### Isolation of Genes Involved with Prostate Cancer

Using the methods disclosed herein, genes associated with prostate cancer were isolated. Various methods of treating and analyzing the cells, including SVM, were utilized to determine the most reliable method for analysis.

Tissues were obtained from patients that had cancer and had undergone prostatectomy. The tissues were processed according to a standard protocol of Affymetrix and gene expression values from 7129 probes on the Affymetrix U95 GeneChip® were recorded for 67 tissues from 26 patients.

Specialists of prostate histology recognize at least three different zones in the prostate: the peripheral zone (PZ), the central zone (CZ), and the transition zone (TZ). In this study, tissues from all three zones are analyzed because previous findings have demonstrated that the zonal origin of the tissue is an important factor influencing the genetic profiling. Most prostate cancers originate in the PZ. Cancers originating in the PZ have worse prognosis than those originating in the TZ. Contemporary biopsy strategies concentrate on the PZ and largely ignored cancer in the TZ. Benign prostate hyperplasia (BPH) is found only in the TZ. BPH is a suitable control used to compare cancer tissues in genetic profiling experiments. BPH is convenient to use as control because it is abundant and easily dissected. However, controls coming from normal tissues microdissected with lasers in the CZ and PZ provide also important complementary controls. The gene expression profile differences have been found to be larger between PZ-G4-G5 cancer and CZ-normal used as control, compared to PZ-normal used as control. A possible explanation comes from the fact that is presence of cancer, even normal adjacent tissues have undergone DNA changes (Malins et al, 2003-2004). Table 1 gives zone properties.

TABLE 1

| Zone | Properties |
|---|---|
| PZ | From apex posterior to base, surrounds transition and |
| | central zones. |
| | Largest zone (70% in young men). |
| | Largest number cancers (60-80%). |
| | Dysplasia and atrophy common in older men. |
| CZ | Surrounds transition zone to angle of urethra to |
| | bladder base. |
| | Second largest zone (25% in young men to 30% at 40 |
| | year old). |
| | 50% of PSA secreting epithelium. |
| | 5-20% of cancers. |
| TZ | Two pear shaped lobes surrounding the proximal urethra. |
| | Smallest zone in young men (less than 5%). |
| | Gives rise to BPH in older men. May expand to the bulk |
| | of the gland. |
| | 10-18% of cancers. |
| | Better cancer prognosis than PZ cancer. |

Classification of cancer determines appropriate treatment and helps determine the prognosis. Cancer develops progressively from an alteration in a cell's genetic structure due to mutations, to cells with uncontrolled growth patterns. Classification is made according to the site of origin, histology (or cell analysis; called grading), and the extent of the disease (called staging).

Prostate cancer specialists classify cancer tissues according to grades, called Gleason grades, which are correlated with the malignancy of the diseases. The larger the grade, the poorer the prognosis (chances of survival). In this study, tissues of grade 3 and above are used. Grades 1 and 2 are more difficult to characterize with biopsies and not very malignant. Grades 4 and 5 are not very differentiated and correspond to the most malignant cancers: for every 10% increase in the percent of grade 4/5 tissue found, there is a concomitant increase in post radical prostatectomy failure rate. Each grade is defined in Table 2.

TABLE 2

| | |
|---|---|
| Grade 1 | Single, separate, uniform, round glands closely packed with |
| | a definite rounded edge limiting the area of the tumor. Sepa- |
| | ration of glands at the periphery from the main collection |
| | by more than one gland diameter indicates a component of at |
| | least grade 2. Uncommon pattern except in the TZ. Almost |
| | never seen in needle biopsies. |
| Grade 2 | Like grade 1 but more variability in gland shape and more |
| | stroma separating glands. Occasional glands show angulated |
| | or distorted contours. More common in TZ than PZ. Patholo- |
| | gists don't diagnose Gleason grades 1 or 2 on prostate |
| | needle biopsies since they are uncommon in the PZ, there is |
| | inter-pathologist variability and poor correlation with radical |
| | prostatectomy. |
| Grade 3 | G3 is the most commonly seen pattern. Variation in size, shape |
| | (may be angulated or compressed), and spacing of glands (may |
| | be separated by >1 gland diameter). Many small glands have |
| | occluded or abortive lumens (hollow areas). There is no |
| | evidence of glandular fusion. The malignant glands infiltrate |
| | between benign glands. |
| Grade 4 | The glands are fused and there is no intervening stroma. |
| Grade 5 | Tumor cells are arranged in solid sheets with no attempts at |
| | gland formation. The presence of Gleason grade 5 and high |
| | percent carcinoma at prostatectomy predicts early death. |

Staging is the classification of the extent of the disease. There are several types of staging methods. The tumor, node, metastases (TNM) system classifies cancer by tumor size (T), the degree of regional spread or lymph node involvement (N), and distant metastasis (M). The stage is determined by the size and location of the cancer, whether it has invaded the prostatic capsule or seminal vesicle, and whether it has metastasized. For staging, MRI is preferred to CT because it permits more accurate T staging. Both techniques can be used in N staging, and they have equivalent accuracy. Bone scintigraphy is used in M staging.

The grade and the stage correlate well with each other and with the prognosis. Adenocarcinomas of the prostate are given two grade based on the most common and second most common architectural patterns. These two grades are added to get a final score of 2 to 10. Cancers with a Gleason score of <6 are generally low grade and not aggressive.

The samples collected included tissues from the Peripheral Zone (PZ); Central Zone (CZ) and Transition Zone (TZ). Each sample potentially consisted of four different cell types: Stomal cells (from the supporting tissue of the prostate, not participating in its function); Normal organ cells; Benign prostatic hyperplasia cells (BPH); Dysplasia cells (cancer precursor stage) and Cancer cells (of various grades indicating the stage of the cancer). The distribution of the samples in Table 3 reflects the difficulty of obtaining certain types of tissues:

TABLE 3

| | | | | | Cancer | Cancer | |
|---|---|---|---|---|---|---|---|
| | Stroma | Normal | BPH | Dysplasia | G3 | G4 | G3 + G4 |
| PZ | 1 | 5 | | 3 | 10 | 24 | 3 |
| CZ | | 3 | | | | | |
| TZ | | | 18 | | | | |

Benign Prostate Hyperplasia (BPH), also called nodular prostatic hyperplasia, occurs frequently in aging men. By the eighth decade, over 90% of males will have prostatic hyperplasia. However, in only a minority of cases (about 10%) will this hyperplasia be symptomatic and severe enough to require surgical or medical therapy. BPH is not a precursor to carcinoma.

It has been argued in the medical literature that TZ BPH could serve as a good reference for PZ cancer. The highest grade cancer (G4) is the most malignant. Part of these experiments are therefore directed towards the separation of BPH vs. G4.

Some of the cells were prepared using laser confocal microscopy (LCM which was used to eliminate as much of the supporting stromal cells as possible and provides purer samples.

Gene expression was assessed from the presence of mRNA in the cells. The mRNA is converted into cDNA and amplified, to obtain a sufficient quantity. Depending on the amount of mRNA that can be extracted from the sample, one or two amplifications may be necessary. The amplification process may distort the gene expression pattern. In the data set under study, either 1 or 2 amplifications were used. LCM data always required 2 amplifications. The treatment of the samples is detailed in Table 4.

TABLE 4

| | 1 amplification | 2 amplifications |
|---|---|---|
| No LCM | 33 | 14 |
| LCM | | 20 |

The end result of data extraction is a vector of 7129 gene expression coefficients.

Gene expression measurements require calibration. A probe cell (a square on the array) contains many replicates of the same oligonucleotide (probe) that is a 25 bases long sequence of DNA. Each “perfect match” (PM) probe is designed to complement a reference sequence (piece of gene). It is associated with a “mismatch” (MM) probe that is identical except for a single base difference in the central position. The chip may contain replicates of the same PM probe at different positions and several MM probes for the same PM probe corresponding to the substitution of one of the four bases. This ensemble of probes is referred to as a probe set. The gene expression is calculated as:
Average Difference=1/pair num Error! Objects cannot be created from editing field codes._{prob set}(PM−MM)

If the magnitude of the probe pair values is not contrasted enough, the probe pair is considered dubious. Thresholds are set to accept or reject probe pairs. Affymetrix considers samples with 40% or over acceptable probe pairs of good quality. Lower quality samples can also be effectively used with the SVM techniques.

A simple “whitening” was performed as pre-processing, so that after pre-processing, the data matrix resembles “white noise”. In the original data matrix, a line of the matrix represented the expression values of 7129 genes for a given sample (corresponding to a particular combination of patient/tissue/preparation method). A column of the matrix represented the expression values of a given gene across the 67 samples. Without normalization, neither the lines nor the columns can be compared. There are obvious offset and scaling problems. The samples were pre-processed to: normalize matrix columns; normalize matrix lines; and normalize columns again. Normalization consists of subtracting the mean and dividing by the standard deviation. A further normalization step was taken when the samples are split into a training set and a test set.

The mean and variance column-wise was computed for the training samples only. All samples (training and test samples) were then normalized by subtracting that mean and dividing by the standard deviation.

Samples were evaluated to determine whether LCM data preparation yields more informative data than unfiltered tissue samples and whether arrays of lower quality contain useful information when processed using the SVM technique.

Two data sets were prepared, one for a given data preparation method (subset 1) and one for a reference method (subset 2). For example, method 1=LCM and method 2=unfiltered samples. Golub's linear classifiers were then trained to distinguish between cancer and normal cases using subset 1 and another classifier using subset 2. The classifiers were then tested on the subset on which they had not been trained (classifier 1 with subset 2 and classifier 2 with subset 1).

If classifier 1 performs better on subset 2 than classifier 2 on subset 1, it means that subset 1 contains more information to do the separation cancer vs. normal than subset 2.

The input to the classifier is a vector of n “features” that are gene expression coefficients coming from one microarray experiment. The two classes are identified with the symbols (+) and (−) with “normal” or reference samples belong to class (+) and cancer tissues to class (−). A training set of a number of patterns {x₁, x₂, . . . xₖ, . . . x_{Error! Objects cannot be created from editing field codes.}} with known class labels {y₁, y₂, . . . yₖ, . . . y_{Error! Objects cannot be created from editing field codes.}}, yₖError! Objects cannot be created from editing field codes. {−1,+1}, is given. The training samples are used to build a decision function (or discriminant function) D(x), that is a scalar function of an input pattern x. New samples are classified according to the sign of the decision function:
D(x)>0 Error! Objects cannot be created from editing field codes.

class (+)
D(x)<0 Error! Objects cannot be created from editing field codes.

class (−)
D(x)=0, decision boundary.
Decision functions that are simple weighted sums of the training patterns plus a bias are called linear discriminant functions.
D(x)=w·x+b,
where w is the weight vector and b is a bias value.

In the case of Golub's classifier, each weight is computed as:
Wᵢ=(Error! Objects cannot be created from editing field codes.ᵢ(+)−Error! Objects cannot be created from editing field codes.ᵢ(−))/(Error! Objects cannot be created from editing field codes.ᵢ(+)+Error! Objects cannot be created from editing field codes.ᵢ(−))
where (Error! Objects cannot be created from editing field codes.ᵢ and Error! Objects cannot be created from editing field codes.ᵢ are the mean and standard deviation of the gene expression values of gene i for all the patients of class (+) or class (−), i=1, . . . n. Large positive wᵢ values indicate strong correlation with class (+) whereas large negative wᵢ values indicate strong correlation with class (−). Thus the weights can also be used to rank the features (genes) according to relevance. The bias is computed as b=−w·Error! Objects cannot be created from editing field codes., where Error! Objects cannot be created from editing field codes.=(Error! Objects cannot be created from editing field codes.(+)+Error! Objects cannot be created from editing field codes.(−))/2.

Golub's classifier is a standard reference that is robust against outliers. Once a first classifier is trained, the magnitude of wᵢ is used to rank the genes. The classifiers are then retrained with subsets of genes of different sizes, including the best ranking genes.

To assess the statistical significance of the results, ten random splits of the data including samples were prepared from either preparation method and submitted to the same method. This allowed the computation of an average and standard deviation for comparison purposes.

Tissue from the same patient was processed either directly (unfiltered) or after the LCM procedure, yielding a pair of microarray experiments. This yielded 13 pairs, including: four G4; one G3+4; two G3; four BPH; one CZ (normal) and one PZ (normal).

For each data preparation method (LCM or unfiltered tissues), the tissues were grouped into two subsets:
Cancer=G4+G3(7 cases)
Normal=BPH+CZ+PZ(6 cases).

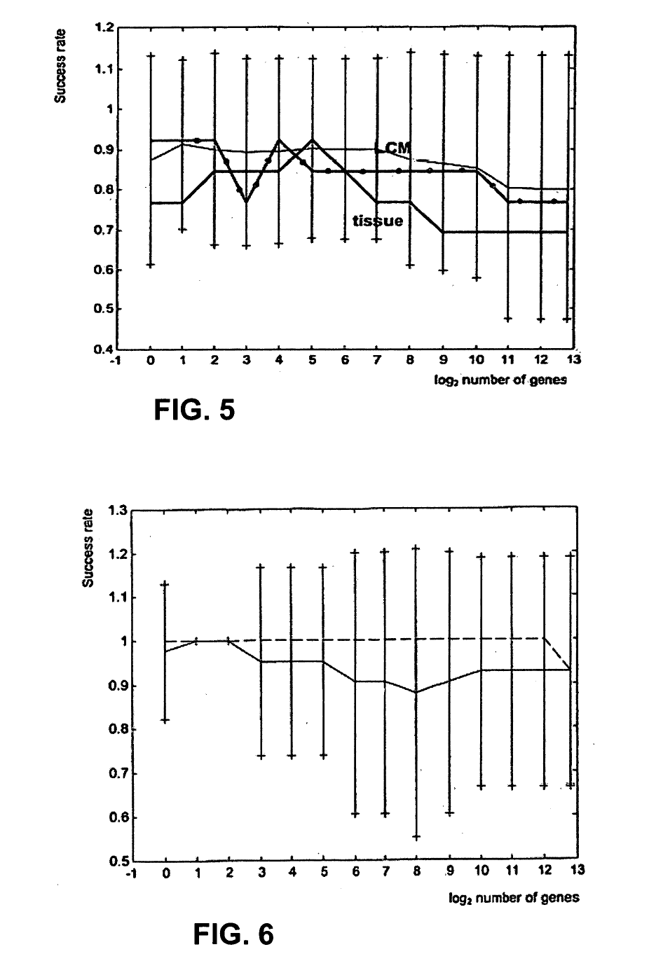

The results are shown in FIG. 5. The large error bars are due to the small size. However, there is an indication that LCM samples are better than unfiltered tissue samples. It is also interesting to note that the average curve corresponding to random splits of the data is above both curves. This is not surprising since the data in subset 1 and subset 2 are differently distributed. When making a random split rather than segregating samples, both LCM and unfiltered tissues are represented in the training and the test set and performance on the test set are better on average.

The same methods were applied to determine whether microarrays with gene expression data rejected by the Affymetrix quality criterion contained useful information by focusing on the problem of separating BPH tissue vs. G4 tissue with a total of 42 arrays (18 BPH and 24 G4).

The Affymetrix criterion identified 17 good quality arrays, 8 BPH and 9 G4. Two subsets were formed:
Subset 1=“good” samples, 8 BPH+9 G4
Subset 2=“mediocre” samples, 10 BPH+15 G4

For comparison, all of the samples were lumped together and 10 random subset 1 containing 8 BPH+9 G4 of any quality were selected. The remaining samples were used as subset 2 allowing an average curve to be obtained. Additionally the subsets were inverted with training on the “mediocre” examples and testing on the “good” examples.

When the mediocre samples are trained, perfect accuracy on the good samples is obtained, whereas training on the good examples and testing on the mediocre yield substantially worse results.

All the BPH and G4 samples were divided into LCM and unfiltered tissue subsets to repeat similar experiments as in the previous Section:
Subset1=LCM samples (5 BPH+6 LCM)
Subset2=unfiltered tissue samples (13 BPH+18 LCM)

There, in spite of the difference in sample size, training on LCM data yields better results. In spite of the large error bars, this is an indication that the LCM data preparation method might be of help in improving sample quality.

BPH vs. G4

The Affymetrix data quality criterion were irrelevant for the purpose of determining the predictive value of particular genes and while the LCM samples seemed marginally better than the unfiltered samples, it was not possible to determine a statistical significance. Therefore, all samples were grouped together and the separation BHP vs. G4 with all 42 samples (18 BPH and 24 G4) was preformed.

To evaluate performance and compare Golub's method with SVMs, the leave-one-out method was used. The fraction of successfully classified left-out examples gives an estimate of the success rate of the various classifiers.

In this procedure, the gene selection process was run 41 times to obtain subsets of genes of various sizes for all 41 gene rankings. One classifier was then trained on the corresponding 40 genes for every subset of genes. This leave-one-out method differs from the “naive” leave-one-out that consists of running the gene selection only once on all 41 examples and then training 41 classifiers on every subset of genes. The naive method gives overly optimistic results because all the examples are used in the gene selection process, which is like “training on the test set”. The increased accuracy of the first method is illustrated in FIG. 6. The method used in the figure is SVM-RFE and the classifier used is an SVM. All SVMs are linear with soft margin parameters C=100 and t=10¹⁴. The dashed line represents the “naive” leave-one-out (loo), which consists in running the gene selection once and performing loo for classifiers using subsets of genes thus derived, with different sizes. The solid line represents the more computationally expensive “true” loo, which consists in running the gene selection 41 times, for every left out example. The left out example is classified with a classifier trained on the corresponding 40 examples for every selection of genes. If f is the success rate obtained (a point on the curve), the standard deviation is computed as sqrt(f(1−f)).

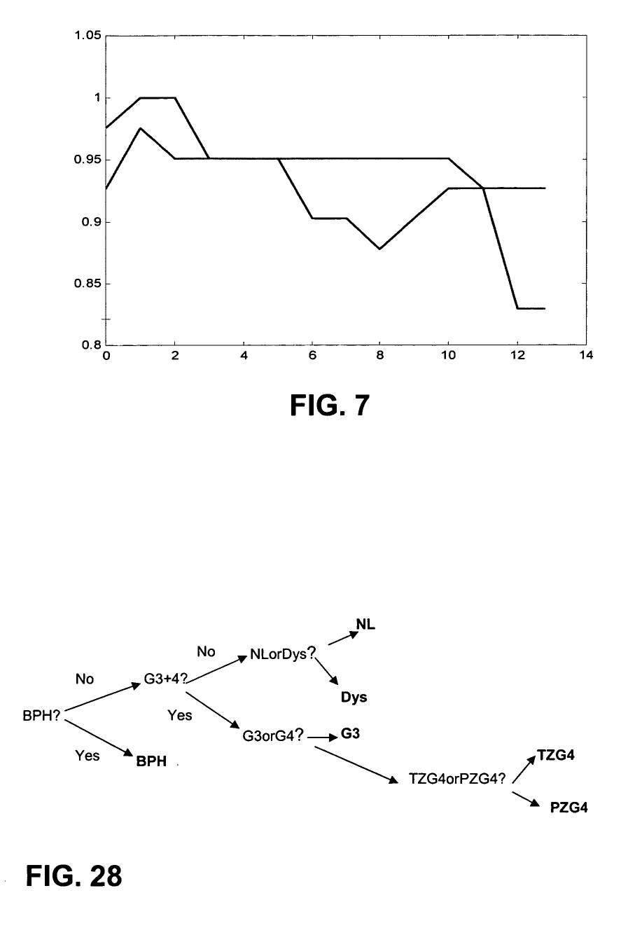

The “true” leave-one-out method was used to evaluate both Golub's method and SVMs. The results are shown in FIG. 7. SVMs outperform Golub's method for the small number of examples. However, the difference is not statistically significant in a sample of this size (1 error in 41 examples, only 85% confidence that SVMs are better).

### EXAMPLE 2

### Analyzing Small Data sets with Multiple Features

Small data sets with large numbers of features present several problems. In order to address ways of avoiding data overfitting and to assess the significance in performance of multivariate and univariate methods, the samples from Example 1 that were classified by Affymetrix as high quality samples were further analyzed. The samples included 8 BPH and 9 G4 tissues. Each microarray recorded 7129 gene expression values. The methods described herein can use the ⅔ of the samples in the BHP/G4 subset that were considered of inadequate quality for use with standard methods.

The first method is used to solve a classical machine learning problem. If only a few tissue examples are used to select best separating genes, these genes are likely to separate well the training examples but perform poorly on new, unseen examples (test examples). Single-feature SVM performs particularly well under these adverse conditions. The second method is used to solve a problem of classical statistics and requires a test that uses a combination of the McNemar criterion and the Wilcoxon test. This test allows the comparison of the performance of two classifiers trained and tested on random splits of the data set into a training set and a test set.

The method of classifying data has been disclosed elsewhere and is repeated here for clarity. The problem of classifying gene expression data can be formulated as a classical classification problem where the input is a vector, a “pattern” of n components is called “features”. F is the n-dimensional feature space. In the case of the problem at hand, the features are gene expression coefficients and patterns correspond to tissues. This is limited to two-class classification problems. The two classes are identified with the symbols (+) and (−). A training set of a number of patterns {x₁, x₂, . . . xₖ, . . . xₚ} with known class labels {y₁ y₂, . . . yₖ, . . . yₚ}, yₖError! Objects cannot be created from editing field codes. {−1,+1}, is given. The training set is usually a subset of the entire data set, some patterns being reserved for testing. The training patterns are used to build a decision function (or discriminant function) D(x), that is a scalar function of an input pattern x. New patterns (e.g. from the test set) are classified according to the sign of the decision function:
D(x)<0 Error! Objects cannot be created from editing field codes.

x Error! Objects cannot be created from editing field codes. class (−)
D(x)>0Error! Objects cannot be created from editing field codes.

x Error! Objects cannot be created from editing field codes. class (+)
D(x)=0, decision boundary.
Decision functions that are simple weighted sums of the training patterns plus a bias are called linear discriminant functions.
D(x)=w·x+b, (2)
where w is the weight vector and b is a bias value.

A data set such as the one used in these experiments, is said to be “linearly separable” if a linear discriminant function can separate it without error. The data set under study is linearly separable. Moreover, there exist single features (gene expression coefficients) that alone separate the entire data set. This study is limited to the use of linear discriminant functions. A subset of linear discriminant functions are selected that analyze data from different points of view:

One approach used multivariate methods, which computed every component of the weight w on the basis of all input variables (all features), using the training examples. For multivariate methods, it does not make sense to intermix features from various rankings as feature subsets are selected for the complementarity of their features, not for the quality of the individual features. The combination is then in selecting the feature ranking that is most consistent with all other ranking, i.e., contains in its top ranking features the highest density of features that appear at the top of other feature rankings. Two such methods were selected:
LDA: Linear Discriminant Analysis, also called Fisher's linear discriminant (see e.g. (Duda, 73)). Fisher's linear discriminant is a method that seeks for w the direction of projection of the examples that maximizes the ratio of the between class variance over the within class variance. It is an “average case” method since w is chosen to maximally separate the class centroids.
SVM: The optimum margin classifier, also called linear Support Vector Machine (linear SVM). The optimum margin classifiers seeks for w the direction of projection of the examples that maximizes the distance between patterns of opposite classes that are closest to one another (margin). Such patterns are called support vector. They solely determine the weight vector w. It is an “extreme case” method as w is determined by the extremes or “borderline” cases, the support vectors.

A second approach, multiple univariate methods, was also used. Such methods computed each component wᵢ of the weight vectors on the basis of the values that the single variable xᵢ takes across the training set. The ranking indicates relevance of individual features. One method was to combine rankings to derive a ranking from the average weight vectors of the classifiers trained on different training sets. Another method was to first create the rankings from the weight vectors of the individual classifiers. For each ranking, a vector is created whose components are the ranks of the features. Such vectors are then averaged and a new ranking is derived from this average vector. This last method is also applicable to the combination of rankings coming from different methods, not necessarily based on the weights of a classifier. Two univariate methods, the equivalents of the multivariate methods were selected:

SF-LDA: Single Feature Linear
Discriminant Analysis:
   wᵢ=(μᵢ(+)−μᵢ(−))/sqrt(p(+)σᵢ(+)²+p(−)σᵢ(−)²) (3)

SF-SVM: Single Feature Support
Vector Machine:
   wᵢ=(sᵢ(+)−sᵢ(−), if sign (sᵢ₍₊₎₋sᵢ₍₋₎)=sign(σᵢ(+)−σᵢ(−)) (4)
wᵢ=0 otherwise.

The parameters μᵢ and σᵢ are the mean and standard deviation of the gene expression values of gene i for all the tissues of class (+) or class (−), i=1, . . . , n. p(+) and p(−) are the numbers of examples of class (+) or class (−).

The single feature Fisher discriminant (SF-LDA) is very similar the method of Golub et al (Golub, 1999). This latter method computes the weights according to wᵢ=(μᵢ(+)−μᵢ(−))/σᵢ(+)+σᵢ(−)). The two methods yield similar results.

Feature normalization plays an important role for the SVM methods. All features were normalized by subtracting their mean and dividing by their standard deviation. The mean and standard deviation are computed on training examples only. The same values are applied to test examples. This is to avoid any use of the test data in the learning process.

The bias value can be computed in several ways. For LDA methods, it is computed as: b=−(m(+)+m(−))/2, where m(+)=w·μ(+) and m(−)=w·μ(−). This way, the decision boundary is in the middle of the projection of the class means on the direction of w. For SVMs, it is computed as b=−(s(+)+s(−))/2, where s(+)=min w.x(+) and s(−)=max w.x(−), the minimum and maximum being taken over all training examples x(+) and x(−) in class (+) and (−) respectively. This way, the decision boundary is in the middle of the projection of the support vectors of either class on the direction of w, which is in the middle of the margin.

The magnitude of the weight vectors of trained classifiers was used to rank features (genes). Intuitively, those features with smallest weight contribute least to the decision function and therefore can be spared.

For univariate methods, such ranking corresponds to ranking features (genes) individually according to their relevance. Subsets of complementary genes that together separate best the two classes cannot be found with univariate methods.

For multivariate methods, each weight wᵢ is a function of all the features of the training examples. Therefore, removing one or several such features affects the optimality of the decision function. The decision function must be recomputed after feature removal (retraining). Recursive Feature Elimination (RFE), the iterative process alternating between two steps is: (1) removing features and (2) retraining, until all features are exhausted. For multiple univariate methods, retraining does not change the weights and is therefore omitted. The order of feature removal defines a feature ranking or, more precisely, nested subsets of features. Indeed, the last feature to be removed with RFE methods may not be the feature that by itself best separates the data set. Instead, the last 2 or 3 features to be removed may form the best subset of features that together separate best the two classes. Such a subset is usually better than a subset of 3 features that individually rank high with a univariate method.

For very small data sets, it is particularly important to assess the statistical significance of the results. Assume that the data set is split into 8 examples for training and 9 for testing. The conditions of this experiment often results in a 1 or 0 error on the test set. A z-test with a standard definition of “statistical significance” (95% confidence) was used. For a test set of size t=9 and a true error rate p=1/9, the difference between the observed error rate and the true error rate can be as large as 17%. The formula ε=z_{η}sqrt(p(1−p)/t), where z_{η}=sqrt(2)erfinv(−2(η−0.5)), η=0.05, was used, where erfinv is the inverse error function, which is tabulated.

The error function is defined as:
erf(x)=Error! Objects cannot be created from editing field codes.exp(−t²)dt.
This estimate assumes i.i.d. errors (where the data used in training and testing were independently and identically distributed), one-sided risk and the approximation of the Binomial law by the Normal law. This is to say that the absolute performance results (question 1) should be considered with extreme care because of the large error bars.

In contrast, it is possible to compare the performance of two classification systems (relative performance, question 2) and, in some cases, assert with confidence that one is better than the other. One of the most accurate tests is the McNemar test, which proved to be particularly well suited to comparing classification systems in a recent benchmark. The McNemar test assesses the significance of the difference between two dependent samples when the variable of interest is a dichotomy. With confidence (1−η) it can be accepted that one classifier is better than the other, using the formula:
(1−η)=0.5+0.5erf(z/sqrt(2)) (5)
where z=εt/sqrt(v); t is the number of test examples, v is the total number of errors (or rejections) that only one of the two classifiers makes, E is the difference in error rate, and erf is the error function
erf(x)=Error! Objects cannot be created from editing field codes.exp(−t²)dt.

This assumes i.i.d. errors, one-sided risk and the approximation of the Binomial law by the Normal law. The comparison of two classification systems and the comparison of two classification algorithms need to be distinguished. The first problem addresses the comparison of the performance of two systems on test data, regardless of how these systems were obtained, i.e., they might have not been obtained by training. This problem arises, for instance, in the quality comparison of two classification systems packaged in medical diagnosis tests ready to be sold. A second problem addresses the comparison of the performance of two algorithms on a given task. It is customary to average the results of several random splits of the data into a training set and a test set of a given size. The proportion of training and test data are varied and results plotted as a function of the training set size. Results are averaged over s=20 different splits for each proportion (only 17 in the case of a training set of size 16, since there are only 17 examples). To compare two algorithms, the same data sets to train and test are used with the two algorithms, therefore obtaining paired experiments. The Wilcoxon signed rank test is then used to evaluate the significance of the difference in performance. The Wilcoxon test tests the null hypothesis two treatments applied to N individuals do not differ significantly. It assumes that the differences between the treatment results are meaningful. The Wilcoxon test is applied as follows: For each paired test i, i=1, . . . s, the difference εᵢ in error rate of the two classifiers trained is computed in the two algorithms to be compared. The test first orders the absolute values of Error! Objects cannot be created from editing field codes.ᵢ the from the least to the greatest. The quantity T to be tested is the sums the ranks of the absolute values of εᵢ over all positive εᵢ. The distribution of T can easily be calculated exactly of be approximated by the Normal law for large values of s. The test could also be applied by replacing εᵢ by the normalized quantity εᵢ/sqrt(vᵢ) used in (5) for the McNemar test, computed for each paired experiment. In this study, the difference in error rate εᵢ is used. The p value of the test is used in the present experiments: the probability of observing more extreme values than T by chance if Hₒ is true: Proba(TestStatistic>Observed T).

If the p value is small, this sheds doubt on Hₒ, which states that the medians of the paired experiments are equal. The alternative hypothesis is that one is larger than the other.

Normalized arrays as provided by Affymetrix were used. No other preprocessing is performed on the overall data set. However, when the data was split into a training set and a test set, the mean of each gene is subtracted over all training examples and divided by its standard deviation. The same mean and standard deviation are used to shift and scale the test examples. No other preprocessing or data cleaning was performed.

It can be argued that genes that are poorly contrasted have a very low signal/noise ratio. Therefore, the preprocessing that divides by the standard deviation just amplifies the noise. Arbitrary patterns of activities across tissues can be obtained for a given gene. This is indeed of concern for unsupervised learning techniques. For supervised learning techniques however, it is unlikely that a noisy gene would by chance separate perfectly the training data and it will therefore be discarded automatically by the feature selection algorithm. Specifically, for an over-expressed gene, gene expression coefficients took positive values for G4 and negative values for BPH. Values are drawn at random with a probability ½ to draw a positive or negative value for each of the 17 tissues. The probability of drawing exactly the right signs for all the tissues is (½)″. The same value exists for an under-expressed gene (opposite signs). Thus the probability for a purely noisy gene to separate perfectly all the BPH from the G4 tissues is p=2(%2)″=1.5.10−5. There are m=7129-5150=1979 presumably noisy genes. If they were all just pure noise, there would be a probability (1−p)ₘ that none of them separate perfectly all the BPH from the G4 tissues. Therefore, a probability 1-(1-p)ₘ-3% that at least one of them does separate perfectly all the BPH from the G4 tissues.

For single feature algorithms, none of a few discarded genes made it to the top, so the risk is irrelevant. For SVM and LDA, there is a higher risk of using a “bad” gene since gene complementarity is used to obtain good separations, not single genes. However, in the best gene list, no gene from the discarded list made it to the top.

Simulations resulting from multiple splits of the data set of 17 examples (8 BPH and 9 G4) into a training set and a test set were run. The size of the training set is varied. For each training set drawn, the remaining data are used for testing.

For number of training examples greater than 4 and less than 16, 20 training sets were selected at random. For 16 training examples, the leave-one-out method was used, in that all the possible training sets obtained by removing 1 example at a time (17 possible choices) were created. The test set is then of size 1. Note that the test set is never used as part of the feature selection process, even in the case of the leave-one-out method.

For 4 examples, all possible training sets containing 2 examples of each class (2 BPH and 2 G4), were created and 20 of them were selected at random.

For SVM methods, the initial training set size is 2 examples, one of each class (1 BPH and 1 G4). The examples of each class are drawn at random. The performance of the LDA methods cannot be computed with only 2 examples, because at least 4 examples (2 of each class) are required to compute intraclass standard deviations. The number of training examples is incremented by steps of 2.

Overall, SF-SVM performs best, with the following four quadrants distinguished. Table 5 shows the best performing methods of feature selection/classification.

TABLE 5

| | Num. Ex. | |
|---|---|---|
| Num. Genes | small | large |
| Large | SF-SVM is best; single | Multivariate methods may |
| | feature methods (SF-SVM | be best; differences not |
| | and SF-LDA) outperform | statistically significant. |
| | multivariate methods | |
| | (SVM and LDA). | |
| Small | SF-LDA is best; LDA is | LDA performs worst; un- |
| | worst; single feature | clear whether single |
| | methods outperform multi- | feature methods perform |
| | variate methods. | better; SF-SVM may have |
| | | an advantage. |

The choice of wᵢ=0 (the coefficient used by Golub et al.) for negative margin genes in SF-SVM corresponds to an implicit pre-selection of genes and partially explains why SF-SVM performs do well for large numbers of genes. In fact, no genes are added beyond the total number of genes that separate perfectly G4 from BPH.

All methods were re-run using the entire data set. The top ranked genes are presented in Tables 6-9. Having determined that the SVM method provided the most compact set of features to achieve 0 leave-one-out error and that the SF-SVM method is the best and most robust method for small numbers of training examples, the top genes found by these methods were researched in the literature. Most of the genes have a connection to cancer or more specifically to prostate cancer.

Table 6 shows the top ranked genes for SF LDA using 17 best BHP/G4.

TABLE 6

| Rank | GAN | EXP | Description |
|---|---|---|---|
| 10 | X83416 | −1 | H. sapiens PrP gene |
| 9 | U50360 | −1 | Human calcium calmodulin-dependent protein |
| | | | kinase II gamma mRNA |
| 8 | U35735 | −1 | Human RACH1 (RACH1) mRNA |
| 7 | M57399 | −1 | Human nerve growth factor (HBNF-1) mRNA |
| 6 | M55531 | −1 | Human glucose transport-like 5 (GLUT5) |
| | | | mRNA |
| 5 | U48959 | −1 | Human myosin light chain kinase (MLCK) |
| | | | mRNA |
| 4 | Y00097 | −1 | Human mRNA for protein p68 |
| 3 | D10667 | −1 | Human mRNA for smooth muscle myosin |
| | | | heavy chain |
| 2 | L09604 | −1 | Homo sapiens differentiation-dependent A4 |
| | | | protein MRNA |
| 1 | HG1612- | 1 | McMarcks |
| | HT1612 | | |

where GAN = Gene Acession Number; EXP = Expression (−1 = underexpressed in cancer (G4) tissues, +1 = overexpressed in cancer tissues).

Table 7 lists the top ranked genes obtained for LDA using 17 best BHP/G4.

TABLE 7

| Rank | GAN | EXP | Description |
|---|---|---|---|
| 10 | J03592 | 1 | Human ADP/ATP translocase mRNA |
| 9 | U40380 | 1 | Human presenilin I-374 (AD3-212) mRNA |
| 8 | D31716 | −1 | Human mRNA for GC box bindig protein |
| 7 | L24203 | −1 | Homo sapiens ataxia-telangiectasia |
| | | | group D |
| 6 | J00124 | −1 | Homo sapiens 50 kDa type I epidermal |
| | | | keratin gene |
| 5 | D10667 | −1 | Human mRNA for smooth muscle myosin |
| | | | heavy chain |
| 4 | J03241 | −1 | Human transforming growth factor-beta |
| | | | 3 (TGF-beta3) MRNA |
| 3 | 017760 | −1 | Human laminin S B3 chain (LAMB3) gene |
| 2 | X76717 | −1 | H. sapiens MT-11 mRNA |
| 1 | X83416 | −1 | H. sapiens PrP gene |

Table 8 lists the top ranked genes obtained for SF SVM using 17 best BHP/G4.

TABLE 8

| Rank | GAN | EXP | Description |
|---|---|---|---|
| 10 | X07732 | 1 | Human hepatoma mRNA for serine protease |
| | | | hepsin |
| 9 | J03241 | −1 | Human transforming growth factor-beta 3 |
| | | | (TGF-beta3) |
| 8 | X83416 | −1 | H. sapiens PrP gene |
| 7 | X14885 | −1 | H. sapiens gene for transforming growth |
| | | | factor-beta 3 |
| 6 | U32114 | −1 | Human caveolin-2 mRNA |
| 5 | M16938 | 1 | Human homeo-box c8 protein |
| 4 | L09604 | −1 | H. sapiens differentiation-dependent A4 |
| | | | protein MRNA |
| 3 | Y00097 | −1 | Human mRNA for protein p68 |
| 2 | D88422 | −1 | Human DNA for cystatin A |
| 1 | U35735 | −1 | Human RACH1 (RACH1) mRNA |

Table 9 provides the top ranked genes for SVM using 17 best BHP/G4.

TABLE 9

| Rank | GAN | EXP | Description |
|---|---|---|---|
| 10 | X76717 | −1 | H. sapiens MT-11 mRNA |
| 9 | U32114 | −1 | Human caveolin-2 mRNA |
| 8 | X85137 | 1 | H. sapiens mRNA for kinesin-related |
| | | | protein |
| 7 | D83018 | −1 | Human mRNA for nel-related protein 2 |
| 6 | D10667 | −1 | Human mRNA for smooth muscle myosin |
| | | | heavy chain |
| 5 | M16938 | 1 | Human homeo box c8 protein |
| 4 | L09604 | −1 | Homo sapiens differentiation-dependent |
| | | | A4 protein |
| 3 | HG1612 | 1 | McMarcks |
| 2 | M10943 | −1 | Human metaIlothionein-If gene (hMT-If) |
| 1 | X83416 | −1 | H. sapiens PrP gene |

Using the “true” leave-one-out method (including gene selection and classification), the experiments indicated that 2 genes should suffice to achieve 100% prediction accuracy. The two top genes were therefore more particularly researched in the literature. The results are summarized in Table 11. It is interesting to note that the two genes selected appear frequently in the top 10 lists of Tables 6-9 obtained by training only on the 17 best genes.

Table 10 is a listing of the ten top ranked genes for SVM using all 42 BHP/G4.

TABLE 10

| Rank | GAN | EXP | Description |
|---|---|---|---|
| 10 | X87613 | −1 | H. sapiens mRNA for skeletal muscle |
| | | | abundant |
| 9 | X58072 | −1 | Human hGATA3 mRNA for trans-acting |
| | | | T-cell specific |
| 8 | M33653 | −1 | Human alpha-2 type IV collagen (COL4A2) |
| 7 | S76473 | 1 | trkB [human brain mRNA] |
| 6 | X14885 | −1 | H. sapiens gene for transforming growth |
| | | | factor-beta 3 |
| 5 | S83366 | −1 | region centromeric to t(12; 17) brake- |
| | | | point |
| 4 | X15306 | −1 | H. sapiens NF-H gene |
| 3 | M30894 | 1 | Human T-cell receptor Ti rearranged |
| | | | gamma-chain |
| 2 | M16938 | 1 | Human homeo box c8 protein |
| 1 | U35735 | −1 | Human RACH1 (RACH1) mRNA |

Table 11 provides the findings for the top 2 genes found by SVM using all 42 BHP/G4. Taken together, the expression of these two genes is indicative of the severity of the disease.

TABLE 11

| GAN | Synonyms | Possible function/link to prostate cancer |
|---|---|---|
| M16938 | HOXC8 | Hox genes encode transcriptional regulatory |
| | | proteins that are largely responsible for |
| | | establishing the body plan of all metazoan |
| | | organisms. There are hundreds of papers |
| | | in PubMed reporting the role of HOX genes |
| | | in various cancers. HOXC5 and HOXC8 |
| | | expression are selectively turned on in |
| | | human cervical cancer cells compared to |
| | | normal keratinocytes. Another homeobox gene |
| | | (GBX2) may participate in metastatic pro- |
| | | gression in prostatic cancer. Another HOX |
| | | protein (hoxb-13) was identified as an |
| | | androgen-independent gene expressed in |
| | | adult mouse prostate epithelial cells. The |
| | | authors indicate that this provides a new |
| | | potential target for developing therapeutics |
| | | to treat advanced prostate cancer |
| U35735 | Jk | Overexpression of RACH2 in human tissue |
| | Kidd | culture cells induces apoptosis. RACH1 is |
| | RACH1 | downregulated in breast cancer cell line |
| | RACH2 | MCF-7. RACH2 complements the RAD1 protein. |
| | SLC14A1 | RAM is implicated in several cancers. |
| | UT1 | Significant positive lod scores of 3.19 for |
| | UTE | linkage of the Jk (Kidd blood group) with |
| | | cancer family syndrome (CFS) were obtained. |
| | | CFS gene(s) may possibly be located on |
| | | chromosome 2, where Jk is located. |

Table 12 shows the severity of the disease as indicated by the top 2 ranking genes selected by SVMs using all 42 BPH and G4 tissues.

TABLE 12

| | HOXC8 | HOXC8 |
|---|---|---|
| | Underexpressed | Overexpressed |
| RACH1Overexpressed | Benign | N/A |
| RACH1 Underexpressed | Grade 3 | Grade 4 |

One of the reasons for choosing SF-LDA as a reference method to compare SVMs against is that SF-LDA is similar to one of the gene ranking techniques used by Affymetrix. (Affymetrix uses that p value of the T-test to rank genes.) While not wishing to be bound by any particular theory, it is believed that the null hypothesis to be tested is the equality of the two expected values of the expressions of a given gene for class (+) BPH and class (−) G4. The alternative hypothesis is that the one with largest average value has the largest expected value. The p value is a monotonically varying function of the quantity to be tested:
Tᵢ=(Error! Objects cannot be created from editing field codes.ᵢ(+)−Error! Objects cannot be created from editing field codes._{I}(−))/(Error! Objects cannot be created from editing field codes.ᵢsqrt(1/p(+)+1/p(−))
where (Error! Objects cannot be created from editing field codes.ᵢ(+)−Error! Objects cannot be created from editing field codes._{I}(−) are the means of the gene expression values of gene i for all the tissues of class (+) or class (−), i=1, . . . , n. p(+) and p(−) are the number of examples of class (+) or class (−); Error! Objects cannot be created from editing field codes.ᵢ²=(p(+) Error! Objects cannot be created from editing field codes.ᵢ(+)²+p(−) Error! Objects cannot be created from editing field codes.ᵢ(−)²)/p is the intra-class variance. Up to a constant factor, which does not affect the ranking, Tᵢ is the same criterion as wᵢ in Equation (3) used for ranking features by SF-LDA.

It was pointed out by Affymetrix that the p value may be used as a measure of risk of drawing the wrong conclusion that a gene is relevant to prostate cancer, based on examining the differences in the means. Assume that all the genes with p value lower than a threshold Error! Objects cannot be created from editing field codes. are selected. At most, a fraction Error! Objects cannot be created from editing field codes. of those genes should be bad choices. However, this interpretation is not quite accurate since the gene expression values of different genes on the same chip are not independent experiments. Additionally, this assumes the equality of the variances of the two classes, which should be tested.

There are variants in the definition of Tᵢ that may account for small differences in gene ranking. Another variant of the method is to restrict the list of genes to genes that are overexpressed in all G4 tissues and underexpressed in all BPH tissues (or vice versa). For purposes of comparison, a variant of SF-LDA was also applied in which only genes that perfectly separate BPH from G4 in the training data were used. This variant performed similarly to SF-LDA for small numbers of genes (as it is expected that a large fraction of the genes ranked high by SF-LDA also separate perfectly the training set). For large numbers of genes, it performed similarly to SF-SVM (all genes that do not separate perfectly the training set get a weight of zero, all the others are selected, like for SF-SVM). But it did not perform better than SF-SVM, so it was not retained.

Another technique that Affymetrix uses is clustering, and more specifically Self Organizing Maps (SOM). Clustering can be used to group genes into clusters and define “super-genes” (cluster centers). The super-genes that are over-expressed for G4 and underexpressed for BPH examples (or vice versa) are identified (visually). Their cluster members are selected. The intersection of these selected genes and genes selected with the T-test is taken to obtain the final gene subset.

Clustering is a means of regularization that reduces the dimensionality of feature space prior to feature selection. Feature selection is performed on a smaller number of “super-genes”.

In summary, meaningful feature selection can be performed with as few as 17 examples and 7129 features. On this data set, single feature SVM performs the best.

### EXAMPLE 3

### Prostate Cancer Study on Affymetrix Gene Expression Data (09-2004)

A set of Affymetrix microarray GeneChip® experiments from prostate tissues were obtained from Professor Stamey at Stanford University. The data statistics from samples obtained for the prostate cancer study are summarized in Table 13. Preliminary investigation of the data included determining the potential need for normalizations. Classification experiments were run with a linear SVM on the separation of Grade 4 tissues vs. BPH tissues. In a 32×3-fold experiment, an 8% error rate could be achieved with a selection of 100 genes using the multiplicative updates technique (similar to RFE-SVM). Performances without feature selection are slightly worse but comparable. The gene most often selected by forward selection was independently chosen in the top list of an independent published study, which provided an encouraging validation of the quality of the data.

TABLE 13

| Prostate zone | Histological classification | No. of samples |
|---|---|---|
| Central (CZ) | Normal (NL) | 9 |
| | Dysplasia (Dys) | 4 |
| | Grade 4 cancer (G4) | 1 |
| Peripheral (PZ) | Normal (NL) | 13 |
| | Dysplasia (Dys) | 13 |
| | Grade 3 cancer (G3) | 11 |
| | Grade 4 cancer (G4) | 18 |
| Transition (TZ) | Benign Prostate Hyperplasia (BPH) | 10 |
| | Grade 4 cancer (G4) | 8 |
| Total | | 87 |

As controls, normal tissues and two types of abnormal tissues are used in the study: BPH and Dysplasia.

To verify the data integrity, the genes were sorted according to intensity. For each gene, the minimum intensity across all experiments was taken. The top 50 most intense values were taken. Heat maps of the data matrix were made by sorting the lines (experiments) according to zone, grade, and time processed. No correlation was found with zone or grade, however, there was a significant correlation with the time the sample was processed. Hence, the arrays are poorly normalized.

In other ranges of intensity, this artifact is not seen. Various normalization techniques were tried, but no significant improvements were obtained. It has been observed by several authors that microarray data are log-normal distributed. A qqplot of all the log of the values in the data matrix confirms that the data are approximately log-normal distributed. Nevertheless, in preliminary classification experiments, there was not a significant advantage of taking the log.

Tests were run to classify BPH vs. G4 samples. There were 10 BPH samples and 27 G4 samples. 32×3fold experiments were performed in which the data was split into 3 subsets 32 times. Two of the subsets were used for training while the third was used for testing. The results were averaged. A feature selection was performed for each of the 32×3 data splits; the features were not selected on the entire dataset.

A linear SVM was used for classification, with ridge parameter 0.1, adjusted for each class to balance the number of samples per class. Three feature selection methods were used: (1) multiplicative updates down to 100 genes (MU100); (2) forward selection with approximate gene orthogonalisation up to 2 genes (FS2); and (3) no gene selection (NO).

The data was either raw or after taking the log (LOG). The genes were always standardized (STD: the mean over all samples is subtracted and the result is divided by the standard deviation; mean and stdev are computed on training data only, the same coefficients are applied to test data).

The results for the performances for the BPH vs. G4 separation are shown in Table 14 below, with the standard errors are shown in parentheses. “Error rate” is the average number of misclassification errors; “Balanced errate” is the average of the error rate of the positive class and the error rate of the negative class; “AUC” is the area under the ROC curves that plots the sensitivity (error rate of the positive class, G4) as a function of the specificity (error rate of the negative class, BPH). It was noted that the SVM performs quite well without feature selection, and MU 100 performs similarly, but slightly better. The number of features was not adjusted—100 was chosen arbitrarily.

TABLE 14

| | | | Balanced | |
|---|---|---|---|---|
| Preprocessing | Feat. Select. | Error rate | errate | AUC |
| Log + STD | MU 100 | 8.09 (0.66) | 11.68 (1.09) | 98.93 (0.2) |
| Log + STD | FS 2 | 13.1 (1.1) | 15.9 (1.3) | 92.02 (1.15) |
| Log + STD | No selection | 8.49 (0.71) | 12.37 (1.13) | 97.92 (0.33) |
| STD | No selection | 8.57 (0.72) | 12.36 (1.14) | 97.74 (0.35) |

In Table 14, the good AUC and the difference between the error rate and the balanced error rate show that the bias of the classifier must be optimized to obtained a desired tradeoff between sensitivity and specificity.

Two features are not enough to match the best performances, but do quite well already.

It was determined that features were selected most often with the FS 2 method. The first gene (3480) was selected 56 times, while the second best one (5783) was selected only 7 times. The first one is believed to be relevant to cancer, while the second one has probably been selected for normalization purpose. It is interesting that the first gene (Hs.79389) is among the top three genes selected in another independent study (Febbo-Sellers, 2003).

The details of the two genes are as follows:
Gene 3480: gb:NM_{—}006159.1/DEF=Homo sapiens nel (chicken)-like 2 (NELL2), mRNA./FEA=mRNA/GEN=NELL2/PROD=nel (chicken)-like 2/DB_XREF=gi:5453765/UG=Hs.79389 nel (chicken)-like 2/FL=gb:D83018.1 gb:NM_{—}006159.1
Gene 5783: gb:NM_{—}018843.1/DEF=Homo sapiens mitochondrial carrier family protein(LOC55972), mRNA./FEA=mRNA/GEN=LOC55972/PROD=mitochondrial carrier family protein /DB_XREF=gi:10047121/UG=Hs.172294 mitochondrial carrier family protein /FL=gb:NM_{—}018843.1 gb:AF125531.1.

### EXAMPLE 4

### Prostate Cancer Study from Affymetrix Gene Expression Data (10-2004)

This example is a continuation of the analysis of Example 3 above on the Stamey prostate cancer microarray data. PSA has long been used as a biomarker of prostate cancer in serum, but is no longer useful. Other markers have been studied in immunohistochemical staining of tissues, including p27, Bcl-2, E-catherin and P53. However, to date, no marker has gained use in routine clinical practice.

The gene rankings obtained correlate with those of the Febbo paper, confirming that the top ranking genes found from the Stamey data have a significant intersection with the genes found in the Febbo study. In the top 1000 genes, about 10% are Febbo genes. In comparison, a random ordering would be expected to have less than 1% are Febbo genes.

BPH is not by itself an adequate control. When selecting genes according to how well they separate grade 4 cancer tissues (G4) from BPH, one can find genes that group all non-BPH tissues with the G4 tissues (including normal, dysplasia and grade 3 tissues). However, when BPH is excluded from the training set, genes can be found that correlate well with disease severity. According to those genes, BPH groups with the low severity diseases, leading to a conclusion that BPH has its own molecular characteristics and that normal adjacent tissues should be used as controls.

TZG4 is less malignant than PZG4. It is known that TZ cancer has a better prognosis than PZ cancer. The present analysis provides molecular confirmation that TZG4 is less malignant than PZG4. Further, TZG4 samples group with the less malignant samples (grade 3, dysplasia, normal, or BPH) than with PZG4. This differentiated grouping is emphasized in genes correlating with disease progression (normal<dysplasia<g3<g4) and selected to provide good separation of TZG4 from PZG4 (without using an ordering for TZG4 and PZG4 in the gene selection criterion).

Ranking criteria implementing prior knowledge about disease malignancy are more reliable. Ranking criteria validity was assessed both with p values and with classification performance. The criterion that works best implements a tissue ordering normal<dysplasia<G3<G4 and seeks a good separation TZG4 from PZG4. The second best criterion implements the ordering normal<dysplasia<G3<TZG4<PZG4.

Comparing with other studies may help reducing the risk of overfitting. A subset of 7 genes was selected that ranked high in the present study and that of Febbo et al. 2004. Such genes yield good separating power for G4 vs. other tissues. The training set excludes BPH samples and is used both to select genes and train a ridge regression classifier. The test set includes 10 BPH and 10 G4 samples (½ from the TZ and ½ from the PZ). Success was evaluated with the area under the ROC curve (“AUC”)(sensitivity vs. specificity) on test examples. AUCs between 0.96 and 1 are obtained, depending on the number of genes. Two genes are of special interest (GSTP1 and PTGDS) because they are found in semen and could be potential biomarkers that do not require the use of biopsied tissue.

The choice of the control may influence the findings (normal tissue or BPH). as may the zones from which the tissues originate. The first test sought to separate Grade 4 from BPH. Two interesting genes were identified by forward selection as gene 3480 (NELL2) and gene 5783 (LOC55972). As explained in Example 3, gene 3480 is the informative gene, and it is believed that gene 5783 helps correct local on-chip variations. Gene 3480, which has Unigene cluster id. Hs.79389, is a Nel-related protein, which has been found at high levels in normal tissue by Febbo et al.

All G4 tissues seem intermixed regardless of zone. The other tissues are not used for gene selection and they all fall on the side of G4. Therefore, the genes found characterize BPH, not G4 cancer, such that it is not sufficient to use tissues of G4 and BPH to find useful genes to characterize G4 cancer.

For comparison, two filter methods were used: the Fisher criterion and the shrunken centroid criterion (Tibshirani et al, 2002). Both methods found gene 3480 to be highly informative (first or second ranking). The second best gene is 5309, which has Unigene cluster ID Hs. 100431 and is described as small inducible cytokine B subfamily (Cys-X-Cys motif). This gene is highly correlated to the first one.

The Fisher criterion is implemented by the following routine:
A vector x containing the values of a given feature for all patt_num samples
cl_num classes, k=1, 2, . . . cl_num, grouping the values of x
mu_val(k) is the mean of the x values for class k
var_val(k) is the variance of the x values for class k
patt_per_class(k) is the number of elements of class k
Unbiased_within_var is the unbiased pooled within class variance, i.e., we make a weighted average of var_val(k) with coefficients patt_per_class(k)/(patt_num-cl_num)
Unbiased_between_var=var(mu_val); % Divides by cl_num-1 then Fisher_crit=Unbiased_between_var/Unbiased_within_var

Although the shrunken centroid criterion is somewhat more complicated that the Fisher criterion, it is quite similar. In both cases, the pooled within class variance is used to normalize the criterion. The main difference is that instead of ranking according to the between class variance (that is, the average deviation of the class centroids to the overall centroid), the shrunken centroid criterion uses the maximum deviation of any class centroid to the global centroid. In doing so, the criterion seeks features that well separate at least one class, instead of features that well separate all classes (on average).

The other small other differences are:

A fudge factor is added to Unbiased_within_std=sqrt(Unbiased_within var) to prevent divisions by very small values. The fudge factor is computed as: fudge=mean(Unbiased_within_std); the mean being taken over all the features.
Each class is weighted according to its number of elements cl_elem(k). The deviation for each class is weighted by 1/sqrt(1/cl_elem(k)+1/patt_num). Similar corrections could be applied to the Fisher criterion.

The two criteria are compared using pvalues. The Fisher criterion produces fewer false positive in the top ranked features. It is more robust, however, it also produces more redundant features. It does not find discriminant features for the classes that are least abundant or hardest to separate.

Also for comparison, the criterion of Golub et al., also known as signal to noise ratio, was used. This criterion is used in the Febbo paper to separate tumor vs. normal tissues. On this data that the Golub criterion was verified to yield a similar ranking as the Pearson correlation coefficient. For simplicity, only the Golub criterion results are reported. To mimic the situation, three binary separations were run: (G3+4 vs. all other tissues), (G4 vs. all other tissues), and (G4 vs. BPH). As expected, the first gene selected for the G4 vs. BPH is 3480, but it does not rank high in the G3+4 vs. all other and G4 vs. all other.

Compared to a random ranking, the genes selected using the various criteria applied are enriched in Febbo genes, which cross-validates the two study. For the multiclass criteria, the shrunken centroid method provides genes that are more different from the Febbo genes than the Fisher criterion. For the two-class separations, the tumor vs normal (G3+4 vs others) and the G4 vs. BPH provide similar Febbo enrichment while the G4 vs. all others gives gene sets that depart more from the Febbo genes. Finally, it is worth noting that the initial enrichment up to 1000 genes is of about 10% of Febbo genes in the gene set. After that, the enrichment decreases. This may be due to the fact that the genes are identified by their Unigene Ids and more than one probe is attributed to the same Id. In any case, the enrichment is very significant compared to the random ranking.

A number of probes do not have Unigene numbers. Of 22,283 lines in the Affymetrix data, 615 do not have Unigene numbers and there are only 14,640 unique Unigene numbers. In 10,130 cases, a unique matrix entry corresponds to a particular Unigene ID. However, 2,868 Unigene IDs are represented by 2 lines, 1,080 by 3 lines, and 563 by more than 3 lines. One Unigene ID covers 13 lines of data. For example, Unigene ID Hs.20019, identifies variants of Homo sapiens hemochromatosis (HFE) corresponding to GenBank assession numbers: AF115265.1, NM_{—}000410.1, AF144240.1, AF150664.1, AF149804.1, AF144244.1, AF115264.1, AF144242.1, AF144243.1, AF144241.1, AF079408.1, AF079409.1, and (consensus) BG402460.

The Unigene IDs of the paper of Febbo et al. (2003) were compared using the U95AV2 Affymetrix array and the IDs found in the U133A array under study. The Febbo paper reported 47 unique Unigene IDs for tumor high genes, 45 of which are IDs also found in the U133A array. Of the 49 unique Unigene IDs for normal high genes, 42 are also found in the U133A array. Overall, it is possible to see cross-correlations between the findings. There is a total of 96 Febbo genes that correspond to 173 lines (some genes being repeated) in the current matrix.

Based on the current results, one can either conclude that the “normal” tissues that are not BPH and drawn near the cancer tissues are on their way to cancer, or that BPH has a unique molecular signature that, although it may be considered “normal”, makes it unfit as a control. A test set was created using 10 BPH samples and 10 grade 4 samples. Naturally, all BPH are in the TZ. The grade 4 are ½ in the TZ and ½ in the PZ.

Gene selection experiments were performed using the following filter methods:

(1)—Pearsons correlation coefficient to correlate with disease severity, where disease severity is coded as normal=1, dysplasia=2, grade3=3, grade4=4.

(2)—Fisher's criterion to separate the 4 classes (normal, dysplasia, grade3, grade4) with no consideration of disease severity.

(3)—Fisher's criterion to separate the 3 classes (PZ, CZ, TZ)

(4)—Relative Fisher criterion by computing the ratio of the between class variances of the disease severity and the zones, in an attempt to de-emphasize the zone factor.

(5)—Fisher's criterion to separate 8 classes corresponding to all the combinations of zones and disease severity found in the training data.

(6)—Using the combination of 2 rankings: the ranking of (1) and a ranking by zone for the grade 4 samples only. The idea is to identify genes that separate TZ from PZ cancers that have a different prognosis.

For each experiment, scatter plots were analyzed for the two best selected genes, the heat map of the 50 top ranked genes was reviewed, and p values were compared. The conclusions are as follows:

The Pearson correlation coefficient tracking disease severity (Experiment (1)) gives a similar ranking to the Fisher criterion, which discriminates between disease classes without ranking according to severity. However, the Pearson criterion has slightly better p values and, therefore, may give fewer false positives. The two best genes found by the Pearson criterion are gene 6519, ranked 6^{th} by the Fisher criterion, and gene 9457, ranked 1^{st} by the Fisher criterion. The test set examples are nicely separated, except for one outlier.

The zonal separation experiments were not conclusive because there are only 3 TZ examples in the training set and no example of CZ in the test set. Experiment (3) revealed a good separation of PZ and CZ on training data. TZ was not very well separated. Experiments (4) and (5) did not show very significant groupings. Experiment (6) found two genes that show both disease progression and that TZ G4 is grouped with “less severe diseases” than PZ G4, although that constraint was not enforced. To confirm the latter finding, the distance for the centroids of PZG4 and TZG4 were compared to control samples. Using the test set only (controls are BPH), 63% of all the genes show that TZG4 is closer to the control than PZG4. That number increases to 70% if the top 100 genes of experiment (6) are considered. To further confirm, experiment (6) was repeated with the entire dataset (without splitting between training and test). TZG4 is closer to normal than PZG4 for most top ranked genes. In the first 15 selected genes, 100% have TZG4 closer to normal than PZG4. This finding is significant because TZG4 has better prognosis than PZG4.

Classification experiments were performed to assess whether the appropriate features had been selected using the following setting:

The data were split into a training set and a test set. The test set consists of 20 samples: 10 BPH, 5 TZG4 and 5 PZG4. The training set contains the rest of the samples from the data set, a total of 67 samples (9 CZNL, 4 CZDYS, 1 CZG4, 13 PZNL, 13 PZDYS, 11 PZG3, 13 PZG4, 3 TZG4). The training set does not contain any BPH.

Feature selection was performed on training data only. Classification was performed using linear ridge regression. The ridge value was adjusted with the leave-one-out error estimated using training data only. The performance criterion was the area under the ROC curve (AUC), where the ROC curve is a plot of the sensitivity as a function of the specificity. The AUC measures how well methods monitor the tradeoff sensitivity/specificity without imposing a particular threshold.

P values are obtained using a randomization method proposed by Tibshirani et al. Random “probes” that have a distribution similar to real features (gene) are obtained by randomizing the columns of the data matrix, with samples in lines and genes in columns. The probes are ranked in a similar manner as the real features using the same ranking criterion. For each feature having a given score s, where a larger score is better, a p value is obtained by counting the fraction of probes having a score larger than s. The larger the number of probes, the more accurate the p value.

For most ranking methods, and for forward selection criteria using probes to compute p values does not affect the ranking. For example, one can rank the probes and the features separately for the Fisher and Pearson criteria.

P values measure the probability that a randomly generated probe imitating a real gene, but carrying no information, gets a score larger or equal to s. Considering a single gene, if it has a score of s, the p value test can be used to test whether to reject the hypothesis that it is a random meaningless gene by setting a threshold on the p value, e.g., 0.0. The problem is that many genes of interest (in the present study, N=22,283.) Therefore, it become probable that at least one of the genes having a score larger than s will be meaningless. Considering many genes simultaneously is like doing multiple testing in statistics. If all tests are independent, a simple correction known as the Bonferroni correction can be performed by multiplying the p values by N. This correction is conservative when the test are not independent.

From p values, one can compute a “false discovery rate” as FDR(s)=pvalue(s)*N/r, where r is the rank of the gene with score s, pvalue(s) is the associated p value, N is the total number of genes, and pvalue(s)*N is the estimated number of meaningless genes having a score larger than s. FDR estimates the ratio of the number of falsely significant genes over the number of genes call significant.

Of the classification experiments described above, the method that performed best was the one that used the combined criteria of the different classification experiments. In general, imposing meaningful constraints derived from prior knowledge seems to improve the criteria. In particular, simply applying the Fisher criterion to the G4 vs. all-the-rest separation (G4vsAll) yields good separation of the training examples, but poorer generalization than the more constrained criteria. Using a number of random probes equal to the number of genes, the G4vsAll identifies 170 genes before the first random probe, multiclass Fisher obtains 105 and the Pearson criterion measuring disease progression gets 377. The combined criteria identifies only 8 genes, which may be attributed to the different way in which values are computed. With respect to the number of Febbo genes found in the top ranking genes, G4vsAll has 20, multiclass Fisher 19, Pearson 19, and the combined criteria 8. The combined criteria provide a characterization of zone differentiation. On the other hand, the top 100 ranking genes found both by Febbo and by criteria G4vsAll, Fisher or Pearson have a high chance of having some relevance to prostate cancer. These genes are listed in Table 15.

TABLE 15

| Order Num | Unigene ID | Fisher | Pearson | G4vs ALL | AUC | Description |
|---|---|---|---|---|---|---|
| 12337 | Hs.7780 | 11 | 6 | 54 | 0.96 | cDNA DKFZp56A072 |
| 893 | Hs.226795 | 17 | 7 | 74 | 0.99 | Glutathione S-transferase pi (GSTP1) |
| 5001 | Hs.823 | 41 | 52 | 72 | 0.96 | Hepsin (transmembrance protease, |
| | | | | | | serine 1) (HPN) |
| 1908 | Hs.692 | 62 | 34 | 111 | 0.96 | Tumor-associated calcium signal |
| | | | | | | transducer 1 (TACSTD1) |
| 5676 | Hs.2463 | 85 | 317 | 151 | 1 | Angiopoietin 1 (ANGPT1) |
| 12113 | Hs.8272 | 181 | 93 | 391 | 1 | Prostaglandin D2 synthase (21 kD, |
| | | | | | | brain) (PTGDS) |
| 12572 | Hs.9651 | 96 | 131 | 1346 | 0.99 | RAS related viral oncogene homolog |
| | | | | | | (RRAS) |

Table 15 shows genes found in the top 100 as determined by the three criteria, Fisher, Pearson and G4vsALL, that were also reported in the Febbo paper. In the table, Order num is the order in the data matrix. The numbers in the criteria columns indicate the rank. The genes are ranked according to the sum of the ranks of the 3 criteria. Classifiers were trained with increasing subset sizes showing that a test AUC of 1 is reached with 5 genes.

The published literature was checked for the genes listed in Table 15. Third ranked Hepsin has been reported in several papers on prostate cancer: Chen et al. (2003) and Febbo et al. (2003) and is picked up by all criteria. Polymorphisms of second ranked GSTP 1 (also picked by all criteria) are connected to prostate cancer risk (Beer et al, 2002). The fact that GSTP1 is found in semen (Lee (1978)) makes it a potentially interesting marker for non-invasive screening and monitoring. The clone DKFZp564A072, ranked first, is cited is several gene expression studies.

Fourth ranked Gene TACSTD1 was also previously described as more-highly expressed in prostate adenocarcinoma (see Lapointe et al, 2004 and references therein). Angiopoietin (ranked fifth) is involved in angiogenesis and known to help the blood irrigation of tumors in cancers and, in particular, prostate cancer (see e.g. Cane, 2003). Prostaglandin D2 synthase (ranked sixth) has been reported to be linked to prostate cancer in some gene expression analysis papers, but more interestingly, prostaglandin D synthase is found in semen (Tokugawa, 1998), making it another biomarker candidate for non-invasive screening and monitoring. Seventh ranked RRAS is an oncogene, so it makes sense to find it in cancer, however, its role in prostate cancer has not been documented.

A combined criterion was constructed for selecting genes according to disease severity NL<DYS<G3<G4 and simultaneously tries to differentiate TZG4 from PZG4 without ordering them. This following procedure was used:
Build an ordering using the Pearson criterion with encoded target vector having values NL=1, DYS=2, G3=3, G4=4 (best genes come last.)
Build an ordering using the Fisher criterion to separate TZG4 from PZG$ (best genes come last.)
Obtain a combined criterion by adding for each gene its ranks obtained with the first and second criterion.
Sort according to the combined criterion (in descending order, best first).
   P values can be obtained for the combined criterion as follows:
Unsorted score vectors for real features (genes) and probes are concatenated for both criteria (Pearson and Fisher).
Genes and probes are sorted together for both criteria, in ascending order (best last).
The combined criterion is obtained by summing the ranks, as described above.
For each feature having a given combined criterion value s (larger values being better), a p value is obtained by counting the fraction of probes a having a combined criterion larger than s.

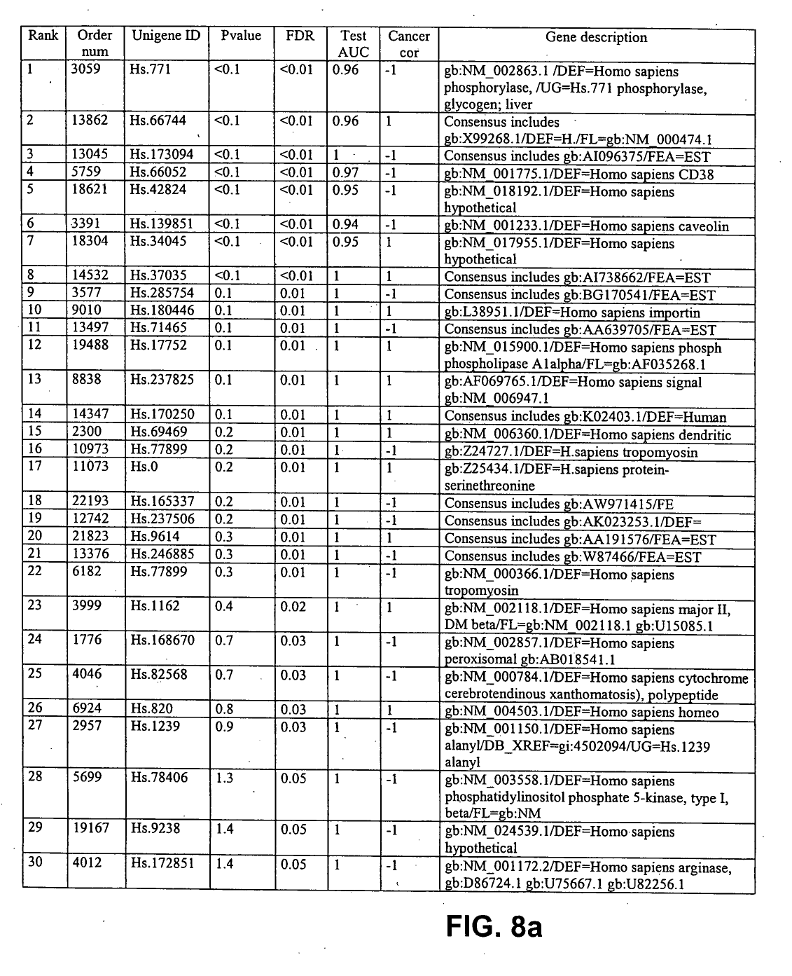

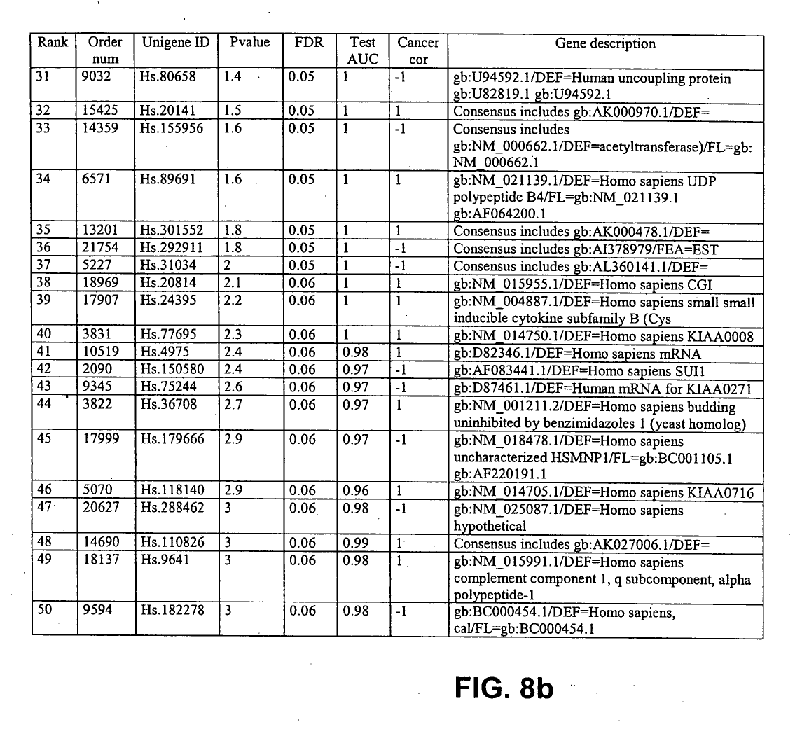

Note that this method for obtaining p values disturbs the ranking, so the ranking that was obtained without the probes in the table in FIG. 8 was used.

A listing of genes obtained with the combined criterion are shown in FIG. 8. The ranking is performed on training data only. “Order num” designates the gene order number in the data matrix; p values are adjusted by the Bonferroni correction; “FDR” indicates the false discovery rate; “Test AUC” is the area under the ROC curve computed on the test set; and “Cancer cor” indicates over-expression in cancer tissues.

From FIGS. 8a-8b, the combined criteria give an AUC of 1 between 8 and 40 genes. This indicates that subsets of up to 40 genes taken in the order of the criteria have a high predictive power. However, genes individually can also be judged for their predictive power by estimating p values. P values provide the probability that a gene is a random meaningless gene. A threshold can be set on that p value, e.g. 0.05.

Using the Bonferroni correction ensures that p values are not underestimated when a large number of genes are tested. This correction penalizes p values in proportion to the number of genes tested. Using 10*N probes (N=number of genes) the number of genes that score higher than all probes are significant at the threshold 0.1. Eight such genes were found with the combined criterion, while 26 genes were found with a p value<1.

It may be useful to filter out as many genes as possible before ranking them in order to avoid an excessive penalty. When the genes were filtered with the criterion that the standard deviation should exceed twice the mean (a criterion not involving any knowledge of how useful this gene is to predict cancer). This reduced the gene set to N′=571, but there were also only 8 genes at the significance level of 0.1 and 22 genes had p value<1.

The 8 first genes found by this method are given in Table 16. Genes over-expressed in cancer are under Rank 2, 7, and 8 (underlined). The remaining genes are under-expressed.

TABLE 16

| Rank | Unigene ID | Description and findings |
|---|---|---|
| 1 | Hs.771 | Phosphorylase, glycogen; liver (Hers disease, |
| | | glycogen storage disease type VI) (PYGL). |
| 2 | Hs.66744 | B-HLH DNA binding protein. H-twist. |
| 3 | Hs.173094 | KIAA1750 |
| 4 | Hs.66052 | CD38 antigen (p45) |
| 5 | Hs.42824 | FLJ10718 hypothetical protein |
| 6 | Hs.139851 | Caveolin 2 (CAV2) |
| 7 | Hs.34045 | FLJ20764 hypothetical protein |
| 8 | Hs.37035 | Homeo box HB9 |

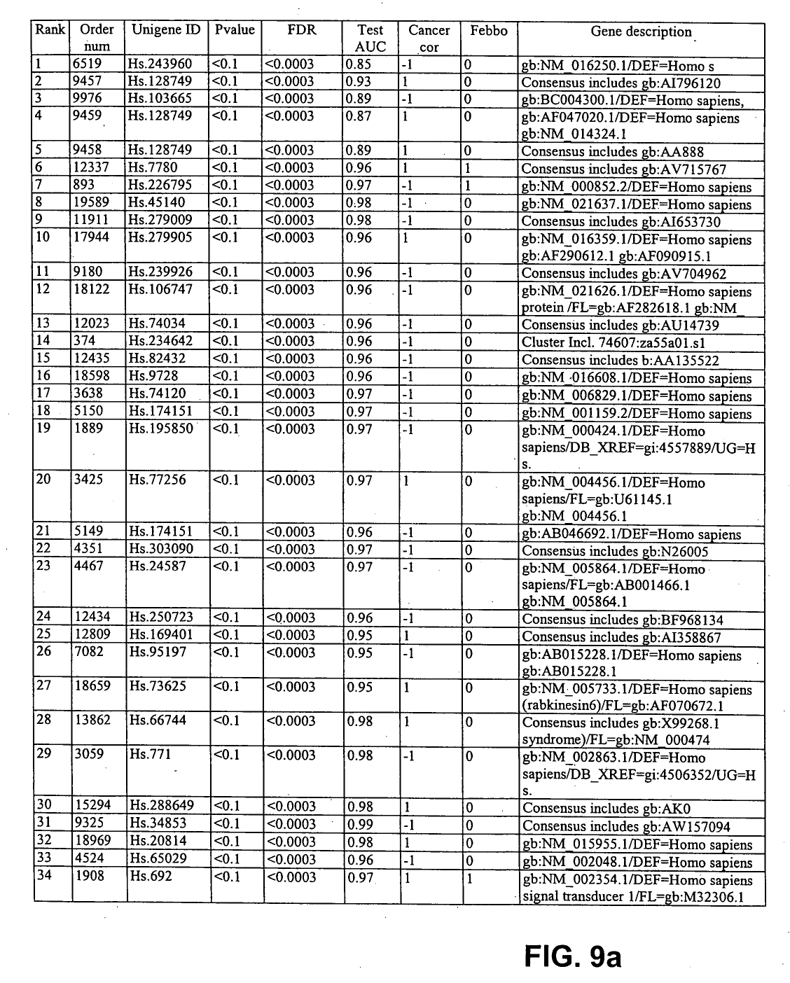

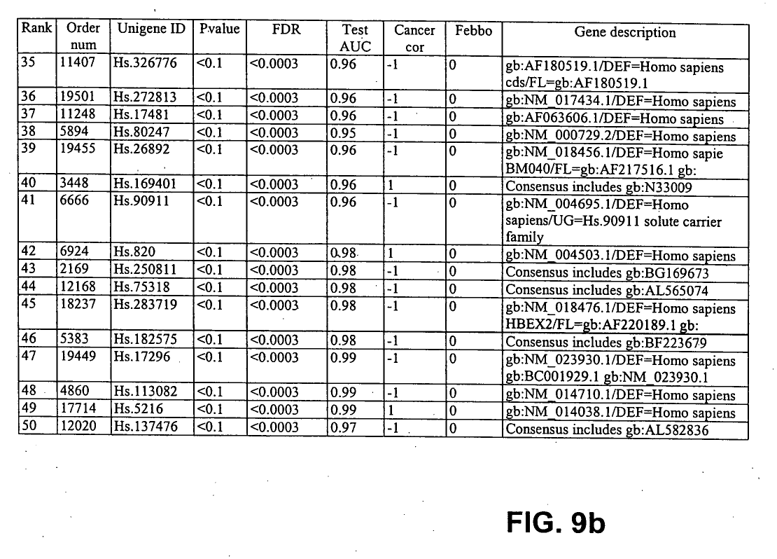

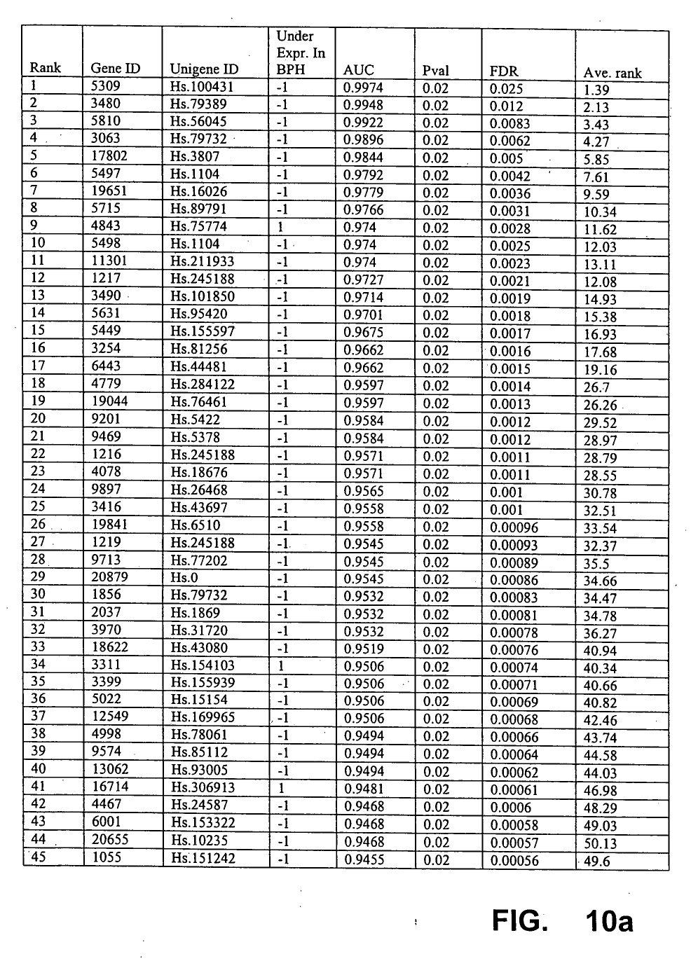

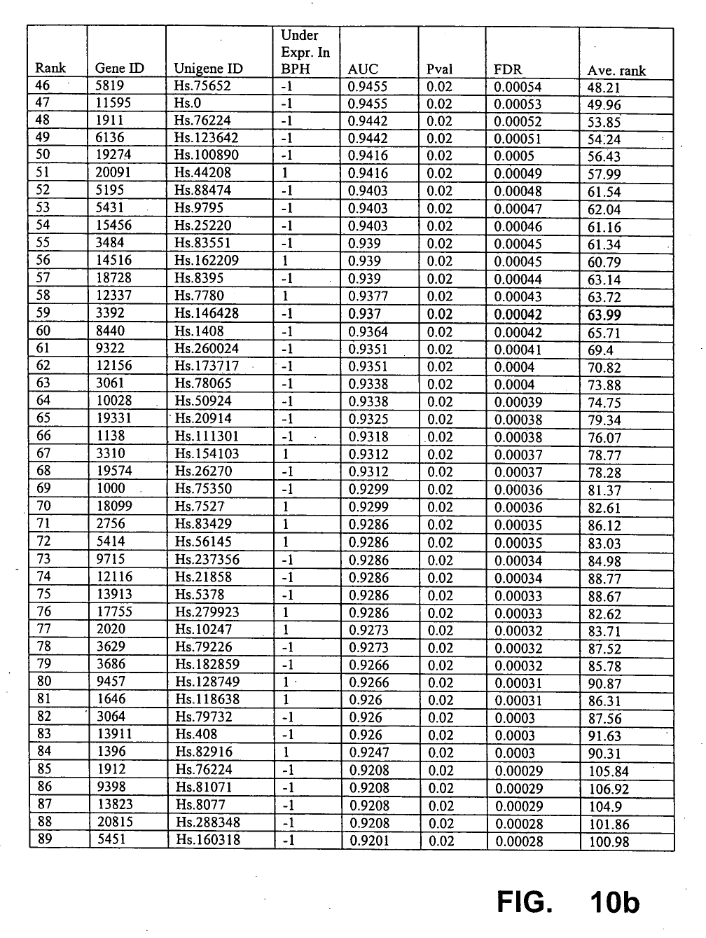

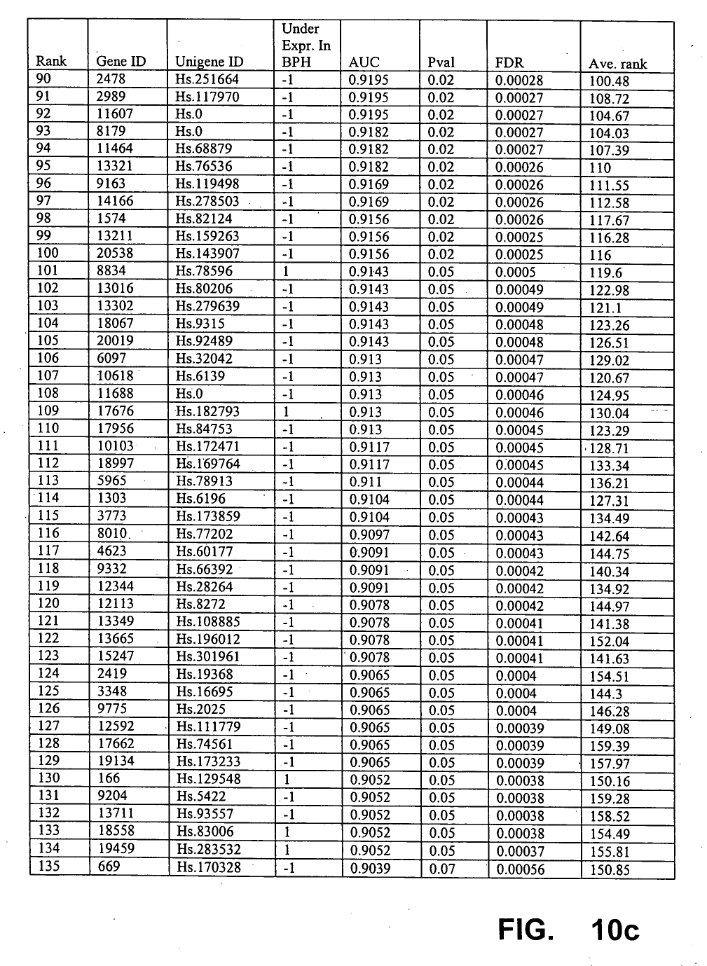

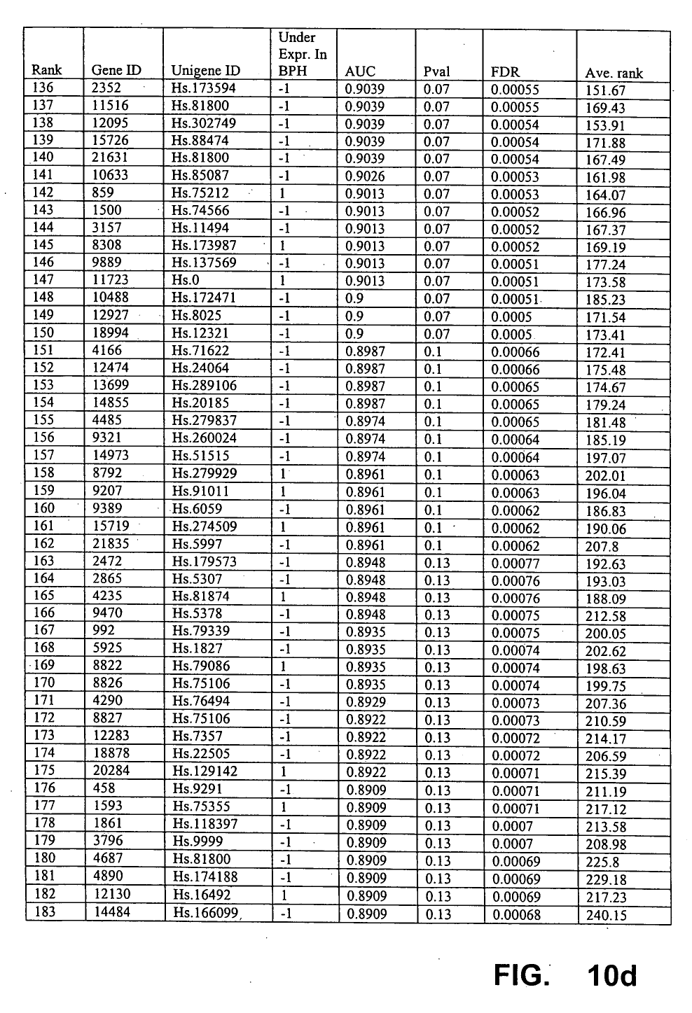

Genes were ranked using the Pearson correlation criterion, see FIG. 9a-9b, with disease progression coded as Normal=1, Dysplasia=2, Grade3=3, Grade4=4. The p values are smaller than in the genes of FIG. 8a-8b, but the AUCs are worse. Three Febbo genes were found, corresponding to genes ranked 6^{th}, 7^{th} and 34^{th}.

The data is rich in potential biomarkers. To find the most promising markers, criteria were designed to implement prior knowledge of disease severity and zonal information. This allowed better separation of relevant genes from genes that coincidentally well separate the data, thus alleviating the problem of overfitting. To further reduce the risk of overfitting, genes were selected that were also found in an independent study (FIG. 8a-8b). Those genes include well-known proteins involved in prostate cancer and some potentially interesting targets.

### EXAMPLE 5

### Prostate Cancer Gene Expression Microarray Data (11-2004)

Several separations of class pairs were performed including “BPH vs. non-BPH” and “tumor (G3+4) vs. all other tissues”. These separations are relatively easy and can be performed with less than 10 genes, however, hundreds of significant genes were identified. The best AUCs (Area under the ROC curve) and BER (balanced error rate) in 10×10-fold cross-validation experiments are on the order of AUC_{BPH}=0.995, BER_{BPH}=5%, AUC_{G34}=0.94, BER_{G34}=9%.

Separations of “G4 vs. all others”, “Dysplasia vs. all others”, and “Normal vs. all others” are less easy (best AUCs between 0.75 and 0.85) and separation of “G3 vs. all others” is almost impossible in this data (AUC around 0.5). With over 100 genes, G4 can be separated from all other tissues with about 10% BER. Hundreds of genes separate G4 from all other tissues significantly, yet one cannot find a good separation with just a few genes.

Separations of “TZG4 vs. PZG4”, “Normal vs. Dysplasia” and “G3 vs. G4” are also hard. 10×10-fold CV yielded very poor results. Using leave-one out CV and under 20 genes, we separated some pairs of classes: ERR_{TZG4/PZG4}≈6%, ERR_{NL/Dys} and ERR_{G3/G4}≈9%. However, due to the small sample sizes, the significance of the genes found for those separations is not good, shedding doubt on the results.

Pre-operative PSA was found to correlate poorly with clinical variables (R²=0.316 with cancer volume, 0.025 with prostate weight, and 0.323 with CAvol/Weight). Genes were found with activity that correlated with pre-operative PSA either in BPH samples or G34 samples or both. Possible connections of those genes were found to cancer and/or prostate in the literature, but their relationship to PSA is not documented. Genes associated to PSA by their description do not have expression values correlated with pre-operative PSA. This illustrates that gene expression coefficients do not necessarily reflect the corresponding protein abundance.

Genes were identified that correlate with cancer volume in G3+4 tissues and with cure/fail prognosis. Neither are statistically significant, however, the gene most correlated with cancer volume has been reported in the literature as connected to prostate cancer. Prognosis information can be used in conjunction with grade levels to determine the significance of genes. Several genes were identified for separating G4 from non-G4 and G3 from G3 in the group the samples of patients with the poor prognosis in regions of lowest expression values.

The following experiments were performed using data consisting of a matrix of 87 lines (samples) and 22283 columns (genes) obtained from an Affymetrix U133A GeneChip®. The distributions of the samples of the microarray prostate cancer study are provided in Table 17.

TABLE 17

| Prostate zone | Histological classification | No. of samples |
|---|---|---|
| Central (CZ) | Normal(NL) | 9 |
| | Dysplasia (Dys) | 4 |
| | Grade 4 cancer (G4) | 1 |
| Peripheral (PZ) | Normal (NL) | 13 |
| | Dysplasia (Dys) | 13 |
| | Grade 3 cancer (G3) | 11 |
| | Grade 4 cancer (G4) | 18 |
| Transition (TZ) | Benign Prostate Hyperplasia (BPH) | 10 |
| | Grade 4 cancer (G4) | 8 |

are used

Genes were selected on the basis of their individual separating power, as measured by the AUC (area under the ROC curve that plots sensitivity vs. specificity).

Similarly “random genes” that are genes obtained by permuting randomly the values of columns of the matrix are ranked. Where N is the total number of genes (here, N=22283, 40 times more random genes than real genes are used to estimate p values accurately (Nᵣ=40*22283). For a given AUC value A, nᵣ(A) is the number of random genes that have an AUC larger than A. The p value is estimated by the fraction of random genes that have an AUC larger than A, i.e.:
Pvalue=(1+nᵣ(A))/Nᵣ

Adding 1 to the numerator avoids having zero p values for the best ranking genes and accounts for the limited precision due to the limited number of random genes. Because the pvalues of a large number of genes are measured simultaneously, correction must be applied to account for this multiple testing. As in the previous example, the simple Bonferroni correction is used:
Bonferroni_pvalue=N*(1+nᵣ(A))/Nᵣ

Hence, with a number of probes that is 40 times the number of genes, the p values are estimated with an accuracy of 0.025.

For a given gene of AUC value A, one can also compute the false discovery rate (FDR), which is an estimate of the ratio of the number of falsely significant genes over the number of genes called significant. Where n(A) is the number of genes found above A, the FDR is computed as the ratio of the p value (before Bonferroni correction) and the fraction of real genes found above A:
FDR=pvalue*N/n(A)=((1+nᵣ(A))*N)/(n(A)*Nᵣ).

Linear ridge regression classifiers (similar to SVMs) were trained with 10×10-fold cross validation, i.e., the data were split 100 times into a training set and a test set and the average performance and standard deviation were computed. In these experiments, the feature selection is performed within the cross-validation loop. That is, a separate featuring ranking is performed for each data split. The number of features are varied and a separate training/testing is performed for each number of features. Performances for each number of features are averaged to plot performance vs. number of features. The ridge value is optimized separately for each training subset and number of features, using the leave-one-out error, which can be computed analytically from the training error. In some experiments, the 10×10-fold cross-validation was done by leave-one-out cross-validation. Everything else remains the same.

Using the rankings obtained for the 100 data splits of the machine learning experiments (also called “bootstraps”), average gene ranks are computed. Average gene rank carries more information in proportion to the fraction of time a gene was always found in the top N ranking genes. This last criterion is sometimes used in the literature, but the number of genes always found in the top N ranking genes appears to grows linearly with N.

The following statistics were computed for cross-validation (10 times 10-fold or leave-one-out) of the machine learning experiments:

AUC mean: The average area under the ROC curve over all data splits.

AUC stdev: The corresponding standard deviation. Note that the standard error obtained by dividing stdev by the square root of the number of data splits is inaccurate because sampling is done with replacements and the experiments are not independent of one another.

BER mean: The average BER over all data splits. The BER is the balanced error rate, which is the average of the error rate of examples of the first class and examples of the second class. This provides a measure that is not biased toward the most abundant class.

BER stdev: The corresponding standard deviation.

Pooled AUC: The AUC obtained using the predicted classification values of all the test examples in all data splits altogether.

Pooled BER: The BER obtained using the predicted classification values of all the test examples in all data splits altogether.

Note that for leave-one-out CV, it does not make sense to compute BER-mean because there is only one example in each test set. Instead, the leave-one-out error rate or the pooled BER is computed.

The first set of experiments was directed to the separation BPH vs. all others.

In previous reports, genes were found to be characteristic of BPH, e.g., gene 3480 (Hs.79389, NELL2).

Of the top 100 genes separating best BPH from all other samples, a very clear separation is found, even with only two genes. In these experiments, gene complementarity was not sought. Rather, genes were selected for their individual separating power. The top two genes are the same as those described in Example 4: gene 3480 (NELL2) and gene 5309 (SCYB13).

Table 18 provides the results of the machine learning experiments for BPH vs. non BPH separation with varying number of features, in the range 2-16 features.

TABLE 18

| | Feat. num. | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 16 | 32 | 64 | 128 |
| 100 * | 98.5 | 99.63 | 99.75 | 99.75 | 99.63 | 99.63 | 99.63 | 99.63 | 99.75 | 99.63 | 99.63 | 99.25 | 96.6 | 92.98 |
| AUC | | | | | | | | | | | | | | |
| 100 * | 4.79 | 2.14 | 1.76 | 1.76 | 2.14 | 2.14 | 2.14 | 2.14 | 1.76 | 2.14 | 2.14 | 3.47 | 10.79 | 17.43 |
| AUCstd | | | | | | | | | | | | | | |
| BER (%) | 9.75 | 5.06 | 5.31 | 5.06 | 5 | 5.19 | 5.31 | 5.31 | 5.31 | 5.44 | 5.19 | 5.85 | 7.23 | 18.66 |
| BERstd (%) | 20.11 | 15.07 | 15.03 | 15.07 | 15.08 | 15.05 | 15.03 | 15.03 | 15.03 | 15.01 | 15.05 | 14.96 | 16.49 | 24.26 |

Very high classification accuracy (as measured by the AUC) is achieved with only 2 genes to provide the AUC above 0.995. The error rate and the AUC are mostly governed by the outlier and the balanced error rate (BER) below 5.44%. Also included is the standard deviation of the 10×10-fold experiment. If the experimental repeats were independent, the standard error of the mean obtained by dividing the standard deviation by 10 could be used as error bar. A more reasonable estimate of the error bar may be obtained by dividing it by three to account for the dependencies between repeats, yielding an error bar of 0.006 for the best AUCs and 5% for BER. For the best AUCs, the error is essentially due to one outlier (1.2% error and 5% balanced error rate). The list of the top 200 genes separating BPH vs. other tissues is given in the table in FIG. 10a-e.

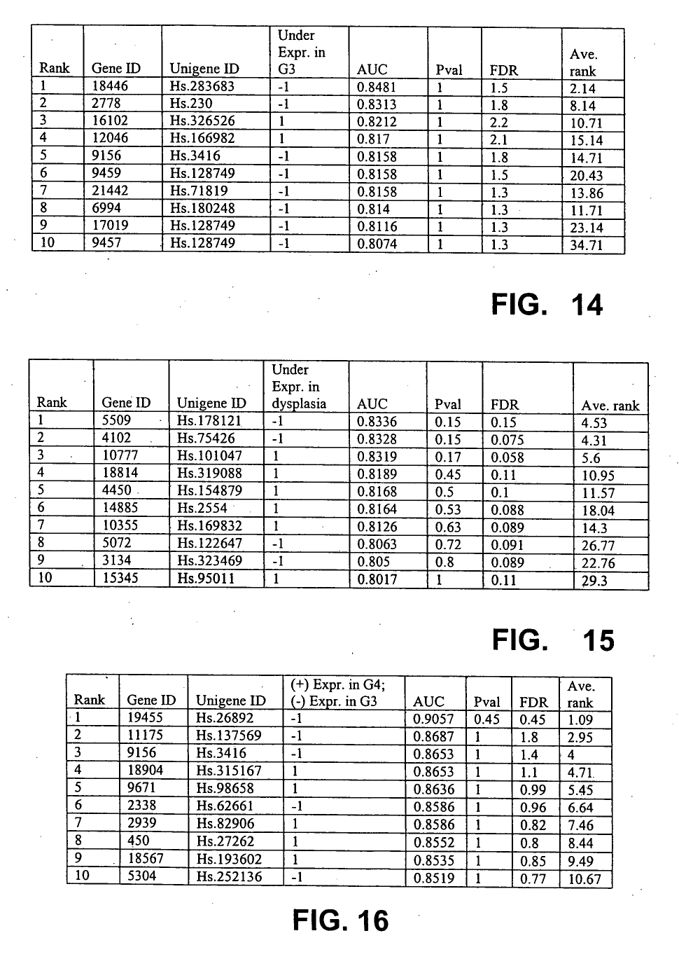

In the tables in FIGS. 10-19, genes are ranked by their individual AUC computed with all the data. The first column is the rank, followed by the Gene ID (order number in the data matrix), and the Unigene ID. The column “Under Expr” is +1 if the gene is underexpressed and −1 otherwise. AUC is the ranking criterion. Pval is the pvalue computed with random genes as explained above. FDR is the false discovery rate. “Ave. rank” is the average rank of the feature when subsamples of the data are taken in a 10×10-fold cross-validation experiment in FIGS. 10-15 and with leave-one-out in FIGS. 16-18.

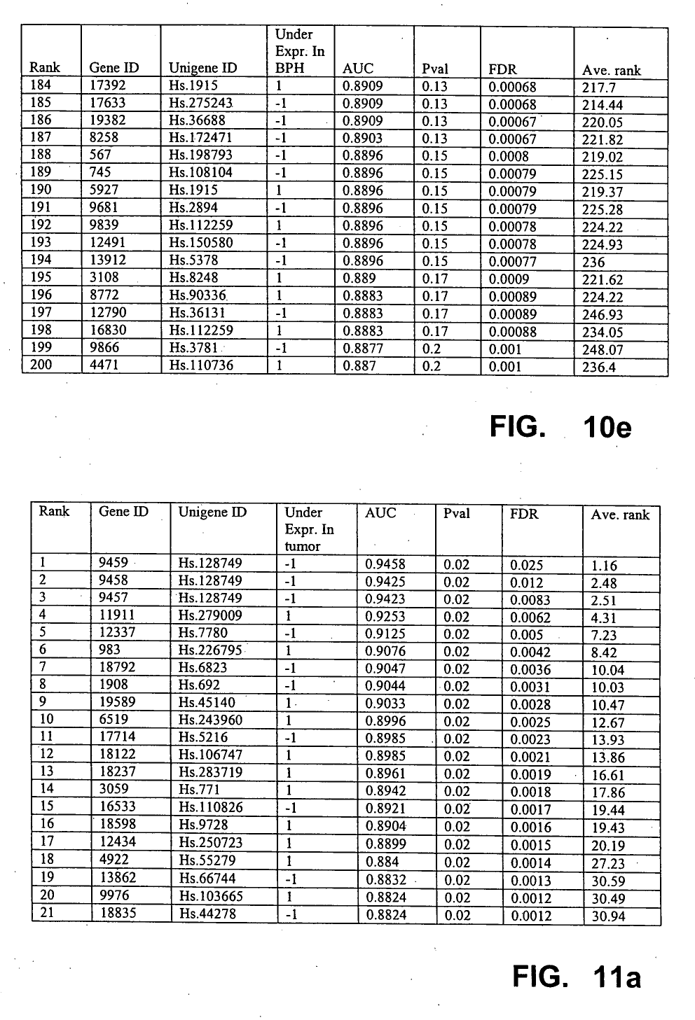

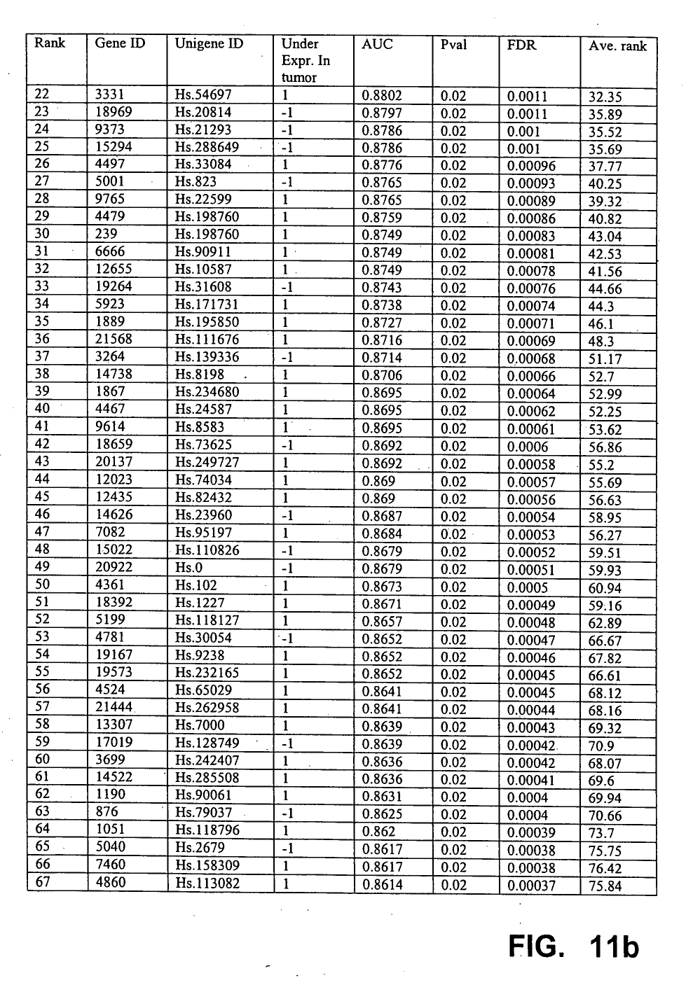

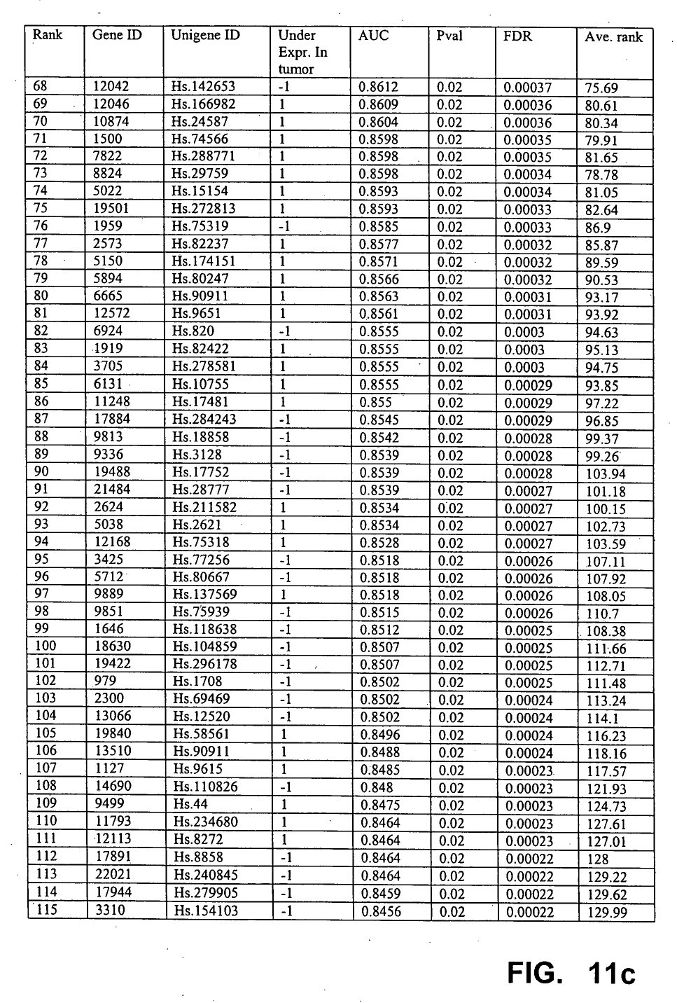

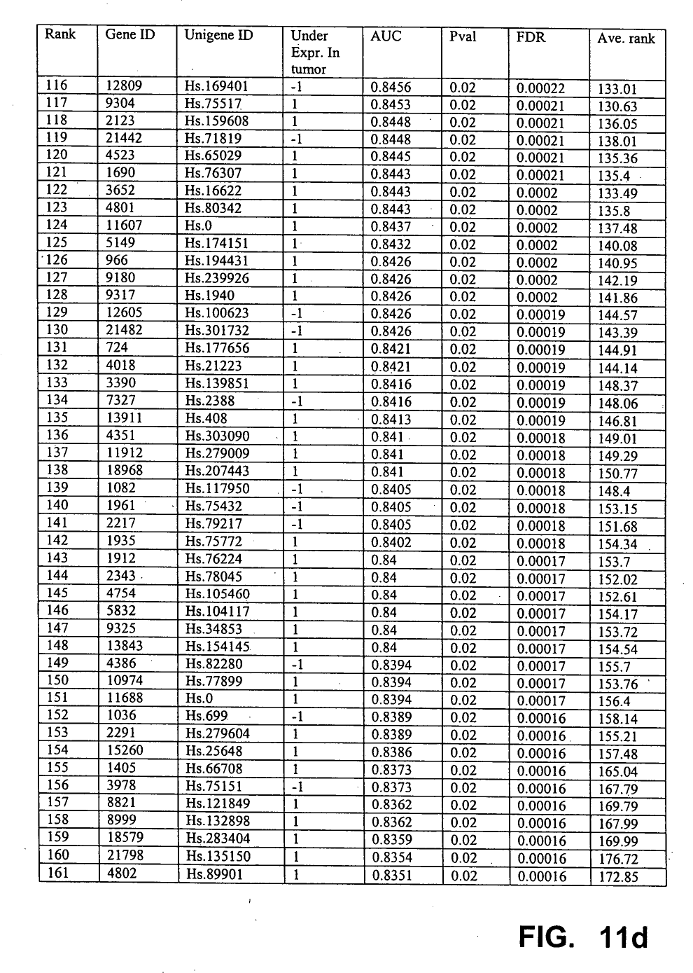

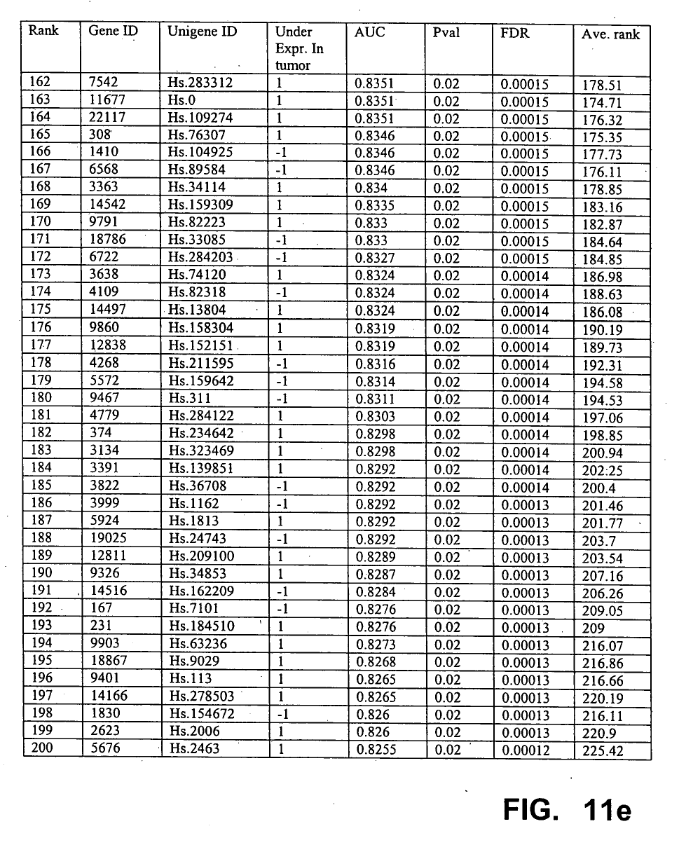

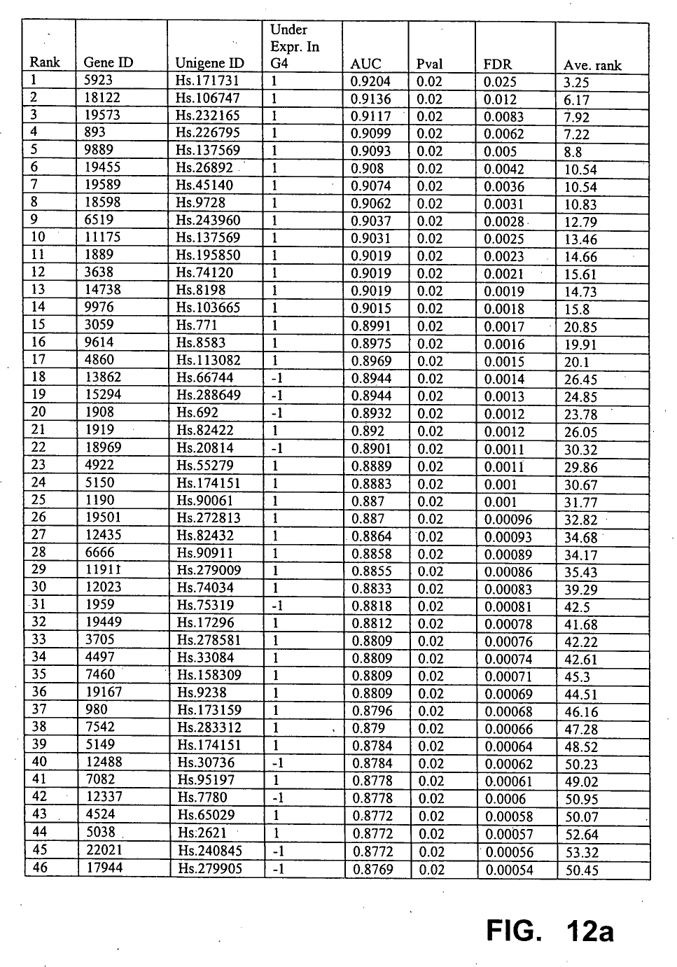

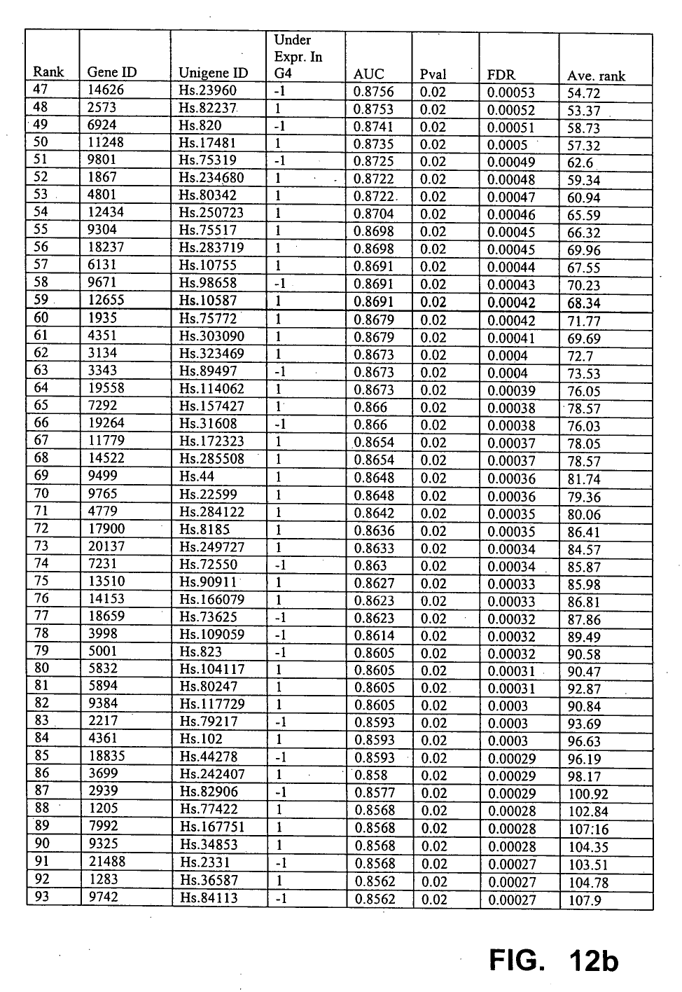

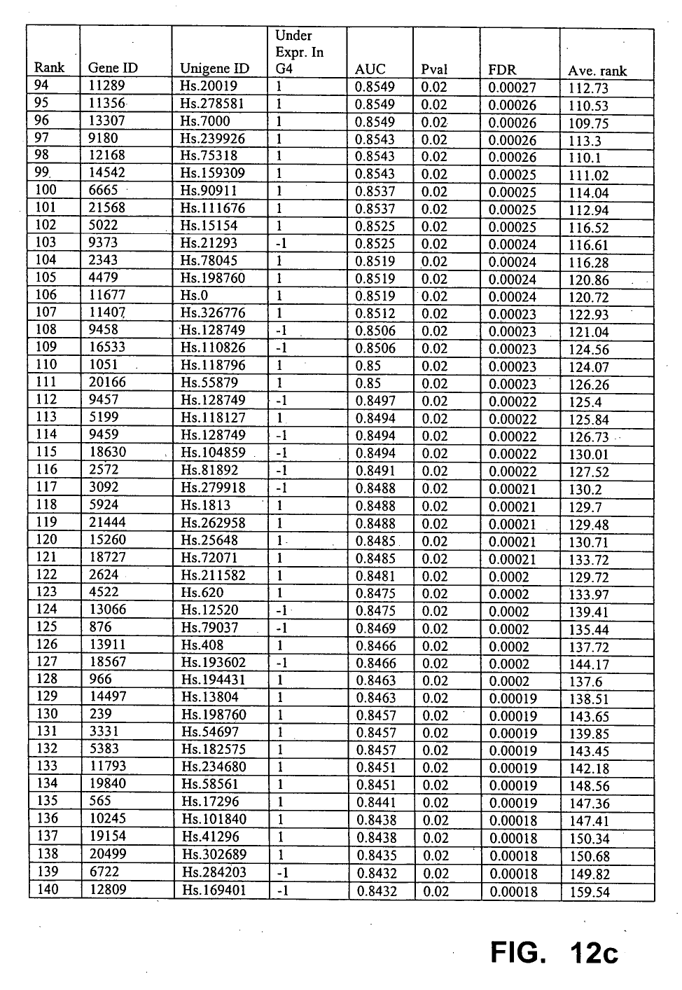

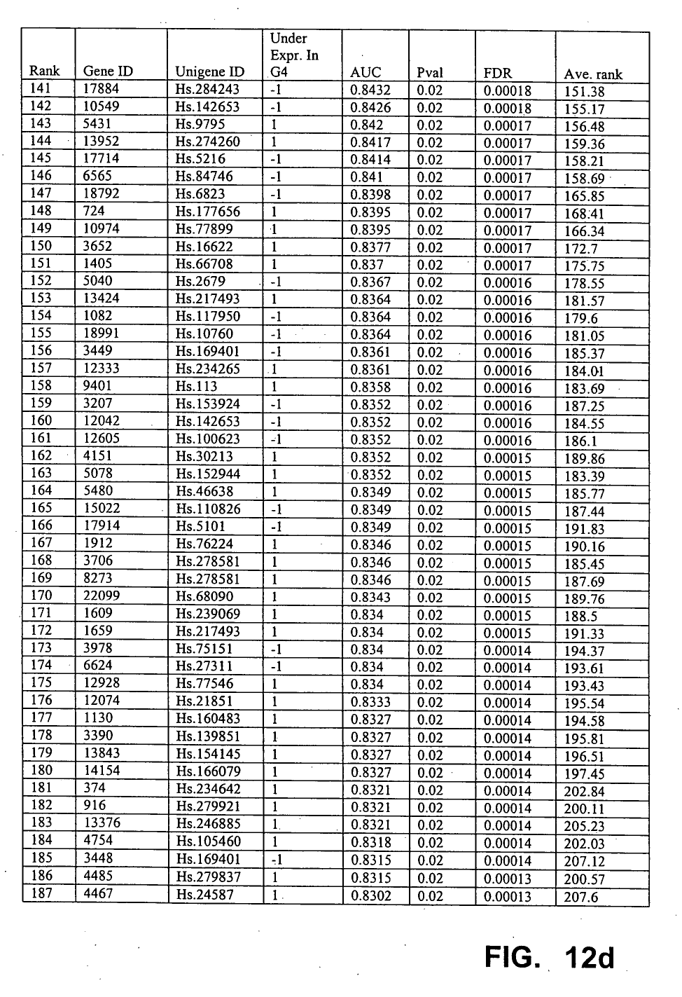

A similar set of experiments was conducted to separate tumors (cancer G3 and G4) from other tissues. The results show that it is relatively easy to separate tumor from other tissues (although not as easy as separating the BPH). The list of the top 200 tumor genes is shown in the table in FIGS. 11a-11e. The three best genes, Gene IDs no. 9457, 9458 and 9459 all have same Unigene ID. Additional description is provided in Table 19 below.

TABLE 19

| Gene | |
|---|---|
| ID | Description |
| 9457 | gb: AI796120 /FEA = EST /DB_XREF = gi: |
| | 5361583 /DB_XREF = est: wh42f03.x1 /CLONE = IMAGE: |
| | 2383421 /UG = Hs.128749 alphamethylacyl-CoA |
| | racemase /FL = gb: AF047020.1 gb: AF158378.1 gb: |
| | NM_014324.1 |
| 9458 | gb: AA888589 /FEA = EST /DB_XREF = gi: |
| | 3004264 /DB_XREF = est: oe68e10.s1 /CLONE = IMAGE: |
| | 1416810 /UG = Hs.128749 alphamethylacyl-CoA |
| | racemase /FL = gb: AF047020.1 gb: AF158378.1 gb: |
| | NM_014324.1 |
| 9459 | gb: AF047020.1 /DEF = Homo sapiens alpha-methylacyl-CoA |
| | racemase mRNA, complete cds. /FEA = mRNA /PROD = alpha- |
| | methylacyl-CoA racemase /DB_XREF = gi: 4204096 /UG = |
| | Hs.128749 alpha-methylacyl-CoA racemase /FL = gb: |
| | AF047020.1 gb: AF158378.1 gb: NM_014324.1 |

This gene has been reported in numerous papers including Luo, et al., Molecular Carcinogenesis, 33(1): 25-35 (January 2002); Luo J, et al., Abstract Cancer Res., 62(8): 2220-6 (2002 Apr. 15).

Table 20 shows the separation with varying number of features for tumor (G3+4) vs. all other tissues.

TABLE 20

| | feat. num. | | | | | | | | | | | | | |
|---|---|---|---|---|---|---|---|---|---|---|---|---|---|---|
| | 1 | 2 | 3 | 4 | 5 | 6 | 7 | 8 | 9 | 10 | 16 | 32 | 64 | 128 |
| 100 * | 92.28 | 93.33 | 93.83 | 94 | 94.33 | 94.43 | 94.1 | 93.8 | 93.43 | 93.53 | 93.45 | 93.37 | 93.18 | 93.03 |
| AUC | | | | | | | | | | | | | | |
| 100 * | 11.73 | 10.45 | 10 | 9.65 | 9.63 | 9.61 | 10.3 | 10.54 | 10.71 | 10.61 | 10.75 | 10.44 | 11.49 | 11.93 |
| AUCstd | | | | | | | | | | | | | | |
| BER (%) | 14.05 | 13.1 | 12.6 | 10.25 | 9.62 | 9.72 | 9.75 | 9.5 | 9.05 | 9.05 | 9.7 | 9.6 | 10.12 | 9.65 |
| BERstd (%) | 13.51 | 12.39 | 12.17 | 11.77 | 9.95 | 10.06 | 10.15 | 10.04 | 9.85 | 10.01 | 10.2 | 10.3 | 10.59 | 10.26 |

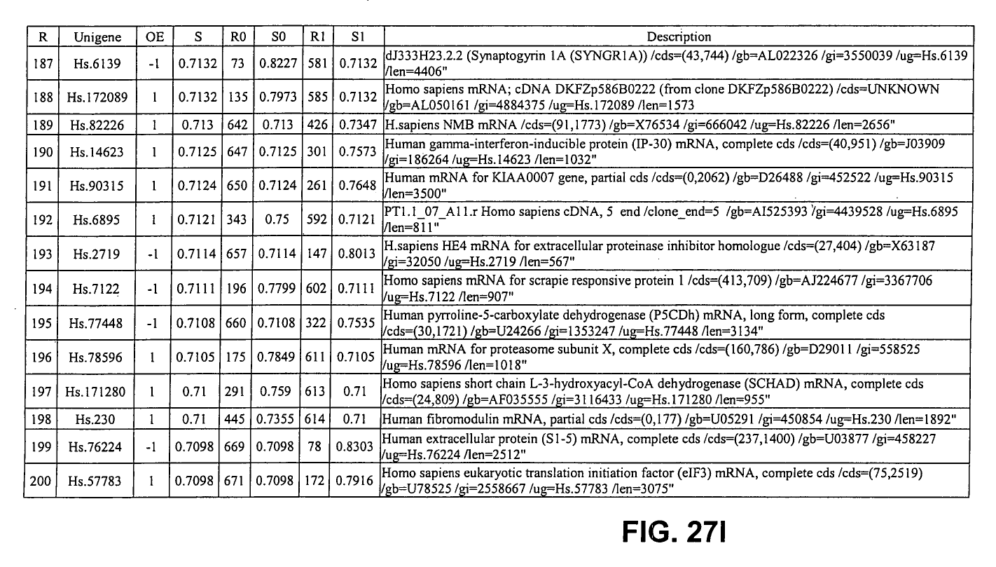

Using the same experimental setup, separations were attempted for G4 from non G4, G3 from non G3, Dysplasia from non-dys and Normal from non-Normal. These separations were less successful than the above-described tests, indicating that G3, dysplasia and normal do not have molecular characteristics that distinguish them easily from all other samples. Lists of genes are provided in FIGS. 12-20. The results suggest making hierarchical decisions as shown in FIG. 28.

FIG. 12a-12e lists the top 200 genes separating Grade 4 prostate cancer (G4) from all others. Table 21 below provides the details for the top two genes of this group.

TABLE 21

| Gene | |
|---|---|
| ID | Description |
| 5923 | gb: NM_015865.1 /DEF = Homo sapiens solute carrier |
| | family 14 (urea transporter), member 1 (Kidd blood |
| | group) (SLC14A1), mRNA. /FEA = mRNA /GEN = |
| | SLC14A1 /PROD = RACH1 /DB_XREF = gi: |
| | 7706676 /UG = Hs.171731 solute carrier family |
| | 14 (urea transporter), member 1 (Kidd blood |
| | group) /FL = gb: U35735.1 gb: NM_015865.1 |
| 18122 | gb: NM_021626.1 /DEF = Homo sapiens serine carboxy- |
| | peptidase 1 precursor protein (HSCP1), mRNA. /FEA = |
| | mRNA /GEN = HSCP1 /PROD = serine carboxypeptidase |
| | 1 precursor protein /DB_XREF = gi: 11055991 /UG = |
| | Hs.106747 serine carboxypeptidase 1 precursor |
| | protein /FL = gb: AF282618.1 gb: NM_021626.1 gb: |
| | AF113214.1 gb: AF265441.1 |

The following provide the gene descriptions for the top two genes identified in each separation:

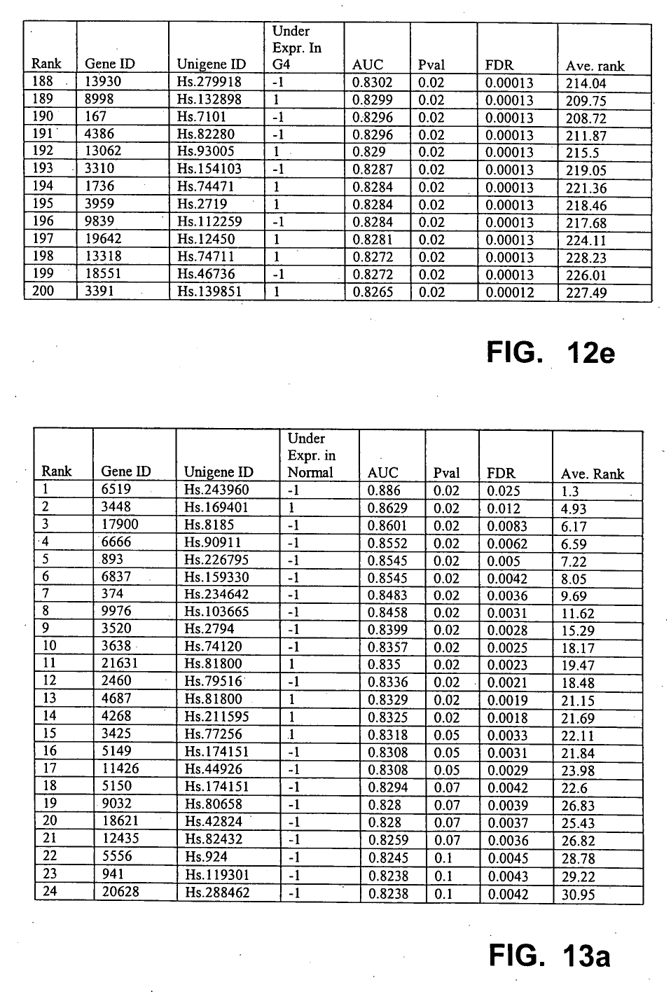

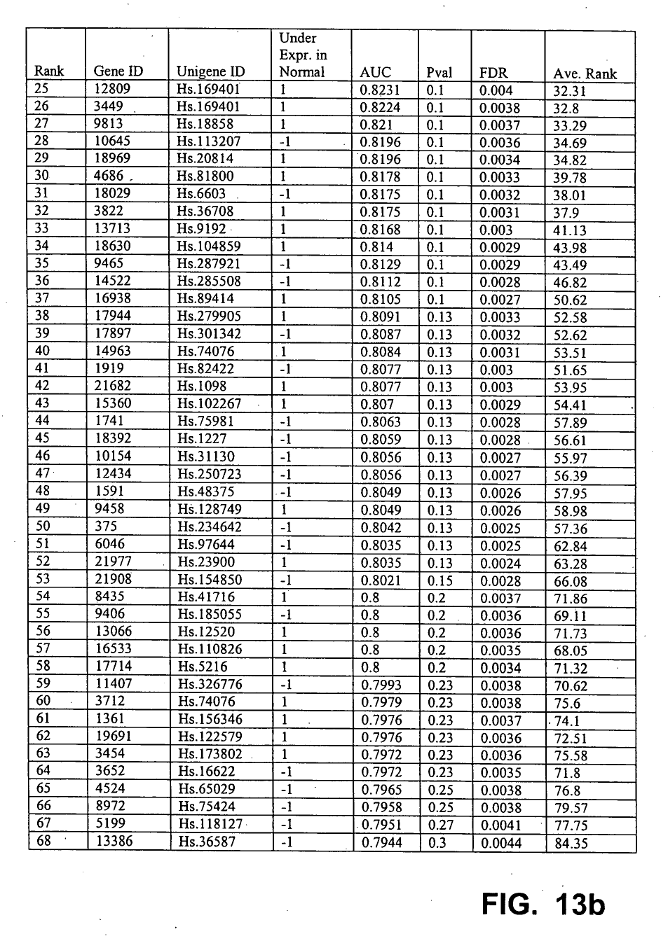

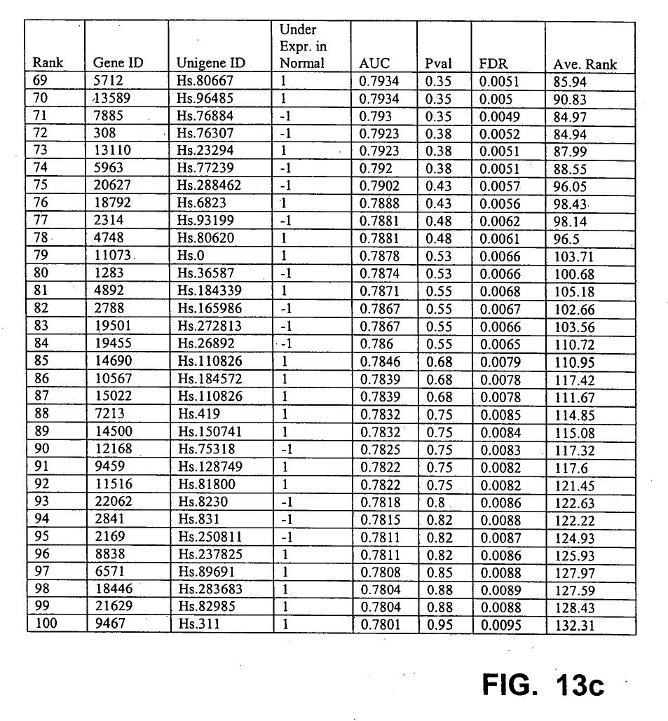

FIG. 13a-13c lists the top 100 genes separating Normal prostate versus all others. The top two genes are described in detail in Table 22.

TABLE 22

| Gene | |
|---|---|
| ID | Description |
| 6519 | gb: NM_016250.1 /DEF = Homo sapiens N-myc downstream- |
| | regulated gene 2 (NDRG2), mRNA. /FEA = mRNA /GEN = |
| | NDRG2 /PROD = KIAA1248 protein /DB_XREF = gi: |
| | 10280619 /UG = Hs.243960 N-myc downstream-regulated |
| | gene 2 /FL = gb: NM_016250.1 gb: AF159092. |
| 3448 | gb: N33009 /FEA = EST /DB_XREF = gi: |
| | 1153408 /DB_XREF = est: yy31f09.s1 /CLONE = IMAGE: |
| | 272873 /UG = Hs.169401 apolipoprotein E /FL = |
| | gb: BC003557.1 gb: M12529.1 gb: K00396.1 gb: NM_000041.1 |

FIG. 14a lists the top 10 genes separating G3 prostate cancer from all others. The top two genes in this group are described in detail in Table 23.

TABLE 23

| Gene | |
|---|---|
| ID | Description |
| 18446 | gb: NM_020130.1 /DEF = Homo sapiens chromosome 8 open |
| | reading frame 4 (C8ORF4), mRNA. /FEA = mRNA /GEN = |
| | C8ORF4 /PROD = chromosome 8 open reading frame |
| | 4 /DB_XREF = gi: 9910147 /UG = Hs.283683 |
| | chromosome 8 open reading frame 4 /FL = gb: |
| | AF268037.1 gb: NM_020130.1 |
| 2778 | gb: NM_002023.2 /DEF = Homo sapiens fibromodulin |
| | (FMOD), mRNA. /FEA = mRNA /GEN = FMOD /PROD = |
| | fibromodulin precursor /DB_XREF = gi: 5016093 /UG = |
| | Hs.230 fibromodulin /FL = gb: NM_002023.2 |

FIG. 15 shows the top 10 genes separating Dysplasia from everything else. Table 24 provides the details for the top two genes listed in FIG. 15.

TABLE 24

| Gene | |
|---|---|
| ID | Description |
| 5509 | gb: NM_021647.1 /DEF = Homo sapiens KIAA0626 gene |
| | product (KIAA0626), mRNA. /FEA = mRNA /GEN = |
| | KIAA0626 /PROD = KIAA0626 gene product /DB_XREF = |
| | gi: 11067364 /UG = Hs.178121 KIAA0626 gene |
| | product /FL = gb: NM_021647.1 gb: AB014526.1 |
| 4102 | gb: NM_003469.2 /DEF = Homo sapiens secretogranin II |
| | (chromogranin C) (SCG2), mRNA. /FEA = mRNA /GEN = |
| | SCG2 /PROD = secretogranin II precursor /DB_XREF = |
| | gi: 10800415 /UG = Hs.75426 secretogranin II |
| | (chromogranin C) /FL = gb: NM_003469.2 gb: M25756.1 |

To support the proposed decision tree of FIG. 28, classifiers are needed to perform the following separations: G3 vs. G4; NL vs. Dys.; and TZG4 vs. PZG4.

Due to the small sample sizes, poor performance was obtained with 10×10-fold cross-validation. To avoid this problem, leave-one-out cross-validation was used instead. In doing so, the average AUC for all repeats cannot be reported because there is only one test example in each repeat. Instead, the leave-one-out error rate and the pooled AUC are evaluated. However, all such pairwise separations are difficult to achieve with high accuracy and a few features.

FIG. 16 lists the top 10 genes separating G3 from G4. Table 25 provides the details for the top two genes listed.

TABLE 25

| Gene | |
|---|---|
| ID | Description |
| 19455 | gb: NM_018456.1 /DEF = Homo sapiens uncharacterized |
| | bone marrow protein BM040 (BM040), mRNA. /FEA = |
| | mRNA /GEN = BM040 /PROD = uncharacterized bone marrow |
| | protein BM040 /DB_XREF = gi: 8922098 /UG = Hs.26892 |
| | uncharacterized bone marrow protein BM040 /FL = gb: |
| | AF217516.1 gb: NM_018456.1 |
| 11175 | gb: AB010153.1 /DEF = Homo sapiens mRNA for p73H, |
| | complete cds. /FEA = mRNA /GEN = p73H /PROD = |
| | p73H /DB_XREF = gi: 3445483 /UG = Hs.137569 tumor |
| | protein 63 kDa with strong homology to p53 /FL = gb: |
| | AB010153.1 |

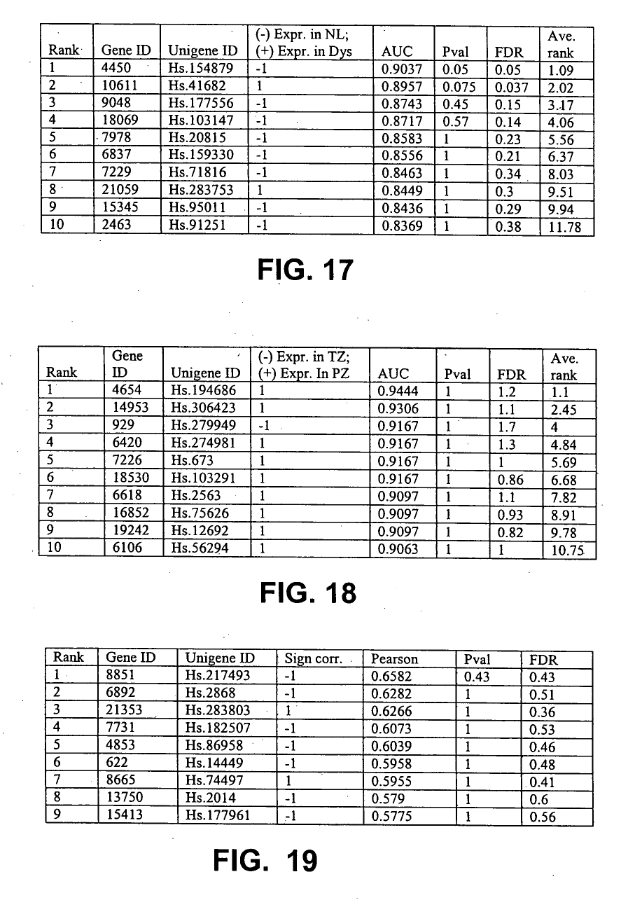

FIG. 17 lists the top 10 genes for separating Normal prostate from Dysplasia. Details of the top two genes for performing this separation are provided in Table 26.

TABLE 26

| Gene | |
|---|---|
| ID | Description |
| 4450 | gb: NM_022719.1 /DEF = Homo sapiens DiGeorge syndrome |
| | critical region gene DGSI (DGSI), mRNA. /FEA = |
| | mRNA /GEN = DGSI /PROD = DiGeorge syndrome critical |
| | region gene DGSIprotein /DB_XREF = gi: 13027629 /UG = |
| | Hs.154879 DiGeorge syndrome critical region gene DGSI /FL = |
| | gb: NM_022719.1 |
| 10611 | gb: U30610.1 /DEF = Human CD94 protein mRNA, complete |
| | cds. /FEA = mRNA /PROD = CD94 protein /DB_XREF = gi: |
| | 1098616 /UG = Hs.41682 killer cell lectin-like receptor |
| | subfamily D, member 1 /FL = gb: U30610.1 gb: NM_002262.2 |

FIG. 18 lists the top 10 genes for separating peripheral zone G4 prostate cancer from transition zone G4 cancer. Table 27 provides the details for the top two genes in this separation.

TABLE 27

| Gene | |
|---|---|
| ID | Description |
| 4654 | gb: NM_003951.2 /DEF = Homo sapiens solute carrier |
| | family 25 (mitochondrial carrier, brain), member 14 |
| | (SLC25A14), transcript variant long, nuclear gene |
| | encoding mitochondrial protein, mRNA. /FEA = |
| | mRNA /GEN = SLC25A14 /PROD = solute carrier |
| | family 25, member 14, isoformUCP5L /DB_XREF = gi: |
| | 6006039 /UG = Hs.194686 solute carrier family 25 |
| | (mitochondrial carrier, brain), member 14 /FL = gb: |
| | AF155809.1 gb: AF155811.1 gb: NM_022810.1 gb: |
| | AF078544.1 gb: NM_003951.2 |
| 14953 | gb: AK002179.1 /DEF = Homo sapiens cDNA FLJ11317 fis, |
| | clone PLACE1010261, moderately similar to SEGREGATION |
| | DISTORTER PROTEIN. /FEA = mRNA /DB_XREF = gi: |
| | 7023899 /UG = Hs.306423 Homo sapiens cDNA FLJ11317 |
| | fis, clone PLACE 1010261, moderately similar to |
| | SEGREGATION DISTORTER PROTEIN |

As stated in an earlier discussion, PSA is not predictive of tissue malignancy. There is very little correlation of PSA and cancer volume (R2=0.316). The R2 was also computed for PSA vs. prostate weight (0.025) and PSA vs. CA/Weight (0.323). PSA does not separate well the samples in malignancy categories. In this data, there did not appear to be any correlation between PSA and prostate weight.

A test was conducted to identify the genes most correlated with PSA, in BPH samples or in G3/4 samples, which were found to be genes 11541 for BPH and 14523 for G3/4. The details for these genes are listed below in Table 28.

TABLE 28

| Gene | |
|---|---|
| ID | Description |
| 11541 | gb: AB050468.1 /DEF = Homo sapiens mRNA for membrane |
| | glycoprotein LIG-1, complete cds. /FEA = mRNA /GEN = |
| | lig-1 /PROD = membrane glycoprotein LIG-1 /DB_XREF = |
| | gi: 13537354 /FL = gb: AB050468.1 |
| 14523 | gb: AL046992 /FEA = EST /DB_XREF = gi: |
| | 5435048 /DB_XREF = est: DKFZp586L0417_r1 /CLONE = |
| | DKFZp586L0417 /UG = Hs.184907 G protein-coupled |
| | receptor 1 /FL = gb: NM_005279.1 |
| 5626 | gb:NM_006200.1 /DEF = Homo sapiens proprotein |
| | convertase subtilisinkexin type 5 (PCSK5), mRNA. /FEA = |
| | mRNA /GEN = PCSK5 /PROD = proprotein convertase |
| | subtilisinkexin type 5 /DB_XREF = gi: 11321618 /UG = |
| | Hs.94376 proprotein convertase subtilisinkexin type |
| | 5 /FL = gb: NM_006200.1 gb: U56387.2 |

Gene 11541 shows no correlation with PSA in G3/4 samples, whereas gene 14523 shows correlation in BPH samples. Thus, 11541 is possibly the result of some overfitting due to the fact that pre-operative PSAs are available for only 7 BPH samples. Gene 14523 appears to be the most correlated gene with PSA in all samples. Gene 5626, also listed in Table 28, has good correlation coefficients (RBPH=0.44, R_{G34}²=0.58).

Reports are found in the published literature indicating that G Protein-coupled receptors such as gene 14523 are important in characterizing prostate cancer. See, e.g. L. L. Xu, et al. Cancer Research 60, 6568-6572, Dec. 1, 2000.

For comparison, genes that have “prostate specific antigen” in their description (none had PSA) were considered:

Gene 4649: gb:NM_{—}001648.1/DEF=Homo sapiens kallikrein 3, (prostate specific antigen) (KLK3), mRNA./FEA=mRNA/GEN=KLK3/PROD=kallikrein 3, (prostate specific antigen)/DB_XREF=gi:4502172/UG=Hs.171995 kallikrein 3, (prostate specific antigen)/FL=gb:BC005307.1 gb:NM_{—}001648.1 gb:U17040.1 gb:M26663.1; and gene 4650: gb:U17040.1/DEF=Human prostate specific antigen precursor mRNA, complete cds./FEA=mRNA/PROD=prostate specific antigen precursor /DB_XREF=gi:595945/UG=Hs.171995 kallikrein 3, (prostate specific antigen) /FL=gb:BC005307.1 gb:NM_{—}001648.1 gb:U17040.1 gb:M26663.1. Neither of these genes had activity that correlates with preoperative PSA.

Another test looked at finding genes whose expression correlate with cancer volume in grade 3 and 4 cancer tissues. However, even the most correlated gene is not found significant with respect to the Bonferroni-corrected pvalue (pval=0.42). The table in FIG. 19 lists the top nine genes most correlated with cancer volume in G3+4 samples. The details of the top gene are provided in Table 29.

TABLE 29

| Gene | |
|---|---|
| ID | Description |
| 8851 | gb: M62898.1 /DEF = Human lipocortin (LIP) 2 pseudogene |
| | mRNA, complete cdslike region. /FEA = mRNA /DB_XREF = |
| | gi: 187147 /UG = Hs.217493 annexin A2 /FL = gb: M62898.1 |

A lipocortin has been described in U.S. Pat. No. 6,395,715 entitled “Uteroglobin gene therapy for epithelial cell cancer”. Using RT-PCR, under-expression of lipocortin in cancer compared to BPH has been reported by Kang J S et al., Clin Cancer Res. 2002 January; 8(1):117-23.

### EXAMPLE 6

### Prostate Cancer Comparative Study of Stamey Data (12-2004)

In this example sets of genes obtained with two different data sets are compared. Both data sets were generated by Dr. Stamey of Stanford University, the first in 2001 using Affymetrix HuGeneFL probe arrays, the second in 2003 using Affymetrix U133A chip. After matching the genes in both arrays, a set of about 2000 common genes. Gene selection was performed on the data of both studies independently, then the gene sets obtained were compared. A remarkable agreement is found. In addition, classifiers were trained on one dataset and tested on the other. In the separation tumor (G3/4) vs. all other tissues, classification accuracies comparable to those obtained in previous reports were obtained by cross-validation on the second study: 10% error can be achieved with 10 genes (on the independent test set of the first study); by cross-validation, there was 8% error. In the separation BPH vs. all other tissues, there was also 10% error with 10 genes. The cross-validation results for BPH were overly optimistic (only one error), however this was not unexpected since there were only 10 BPH samples in the second study. Tables of genes were selected by consensus of both studies.

The 2001 (first) gene set consists of 67 samples from 26 patients. The Affymetrix HuGeneFL probe arrays used have 7129 probes, representing 6500 genes. The composition of the 2001 dataset (number of samples in parenthesis) is summarized in Table 30. Several grades and zones are represented, however, all TZ samples are BPH (no cancer), all CZ samples are normal (no cancer). Only the PZ contains a variety of samples. Also, many samples came from the same tissues.

TABLE 30

| Zone | Histological classification |
|---|---|
| CZ(3) | NL(3) |
| PZ (46) | NL (5) |
| | Stroma(1) |
| | Dysplasia (3) |
| | G3 (10) |
| | G4 (27) |
| TZ(18) | BPH(18) |
| Total 67 | |

The 2003 (second) dataset consists of a matrix of 87 lines (samples) and 22283 columns (genes) obtained from an Affymetrix U133A chip. The distribution of the samples of the microarray prostate cancer study is given in Table 31.

TABLE 31

| Prostate zone | Histological classification | No. of samples |
|---|---|---|
| Central (CZ) | Normal (NL) | 9 |
| | Dysplasia (Dys) | 4 |
| | Grade 4 cancer (G4) | 1 |
| Peripheral (PZ) | Normal (NL) | 13 |
| | Dysplasia (Dys) | 13 |
| | Grade 3 cancer (G3) | 11 |
| | Grade 4 cancer (G4) | 18 |
| Transition (TZ) | Benign Prostate Hyperplasia (BPH) | 10 |
| | Grade 4 cancer (G4) | 8 |

Genes that had the same Gene Accession Number (GAN) in the two arrays HuGeneFL and U133A were selected. The selection was further limited to descriptions that matched reasonably well. For that purpose, a list of common words was created. A good match corresponds to a pair of description having at least a common word, excluding these common words, short word (less that 3 letters) and numbers. The results was a set of 2346 genes.

Because the data from both studies came normalized in different ways, it was re-normalized using the routine provided below. Essentially, the data is translated and scaled, the log is taken, the lines and columns are normalized, the outlier values are squashed. This preprocessing was selected based on a visual examination of the data.

For the 2001 study, a bias=−0.08 was used. For the 2003 study, the bias=0. Visual examination revealed that these value stabilize the variance of both classes reasonably well.

function X=my_normalize(X, bias)

if margin<2, bias=O; end

mini=min(min(X));

maxi=max(max(X));

X=(X-mini)/(maxi-mini)+bias;

idx=find(X<=O);

X(idx)=Inf;

epsi=min(min(X)); X

(idx)=epsi;

X=(X);

Xmed_normalize(X);

X=med_normalize(X′)′;

Xmed_normalize(X);

X=med_normalize(X′)′;

Xtanh(0.1*X);

function X=med_normalize(X)

mu=mean(X,2);

One=ones(size(X,2), 1);

XM=X-mu(:,One);

S=median(abs(XM),2);

X=XM.IS(:,One);

The set of 2346 genes was ranked using the data of both studies independently, with the area under the ROC curve (AUC) being used as the ranking criterion. P values were computed with the Bonferroni correction and False discovery rate (FDR) was calculated.

Both rankings were compared by examining the correlation of the AUC scores. Cross-comparisons were done by selecting the top 50 genes in one study and examining how “enriched” in those genes were the lists of top ranking genes from the other study, varying the number of genes. This can be compared to a random ranking. For a consensus ranking, the genes were ranked according to their smallest score in the two studies.

Reciprocal tests were run in which the data from one study was used for training of the classifier which was then tested on the data from the other study. Three different classifiers were used: Linear SVM, linear ridge regression, and Golub's classifier (analogous to Naïve Bayes). For every test, the features selected with the training set were used. For comparison, the consensus features were also used.

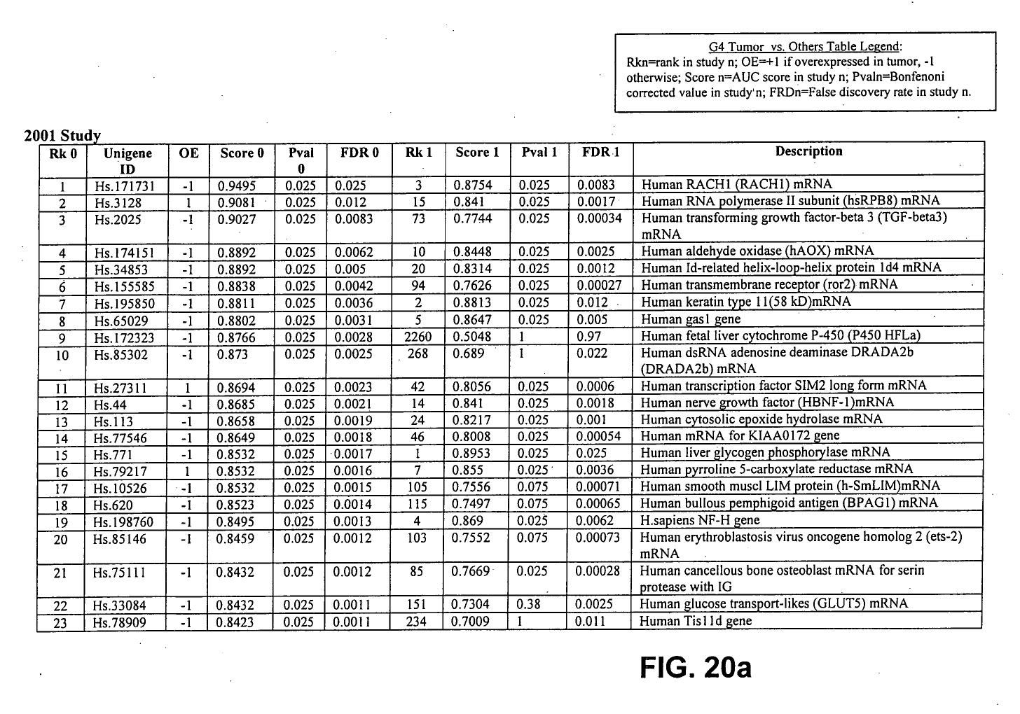

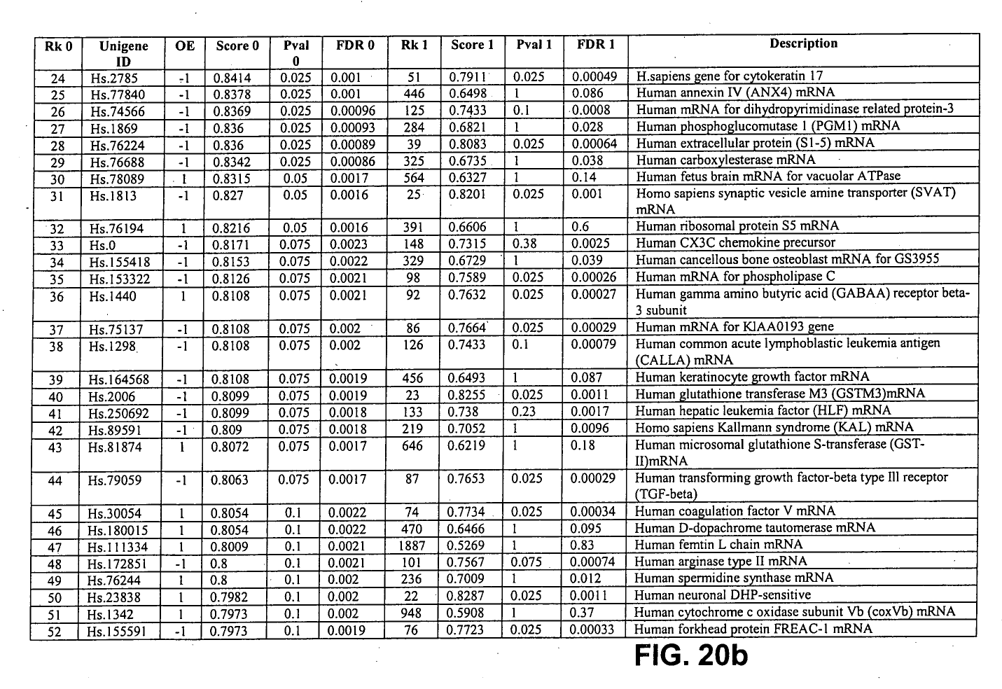

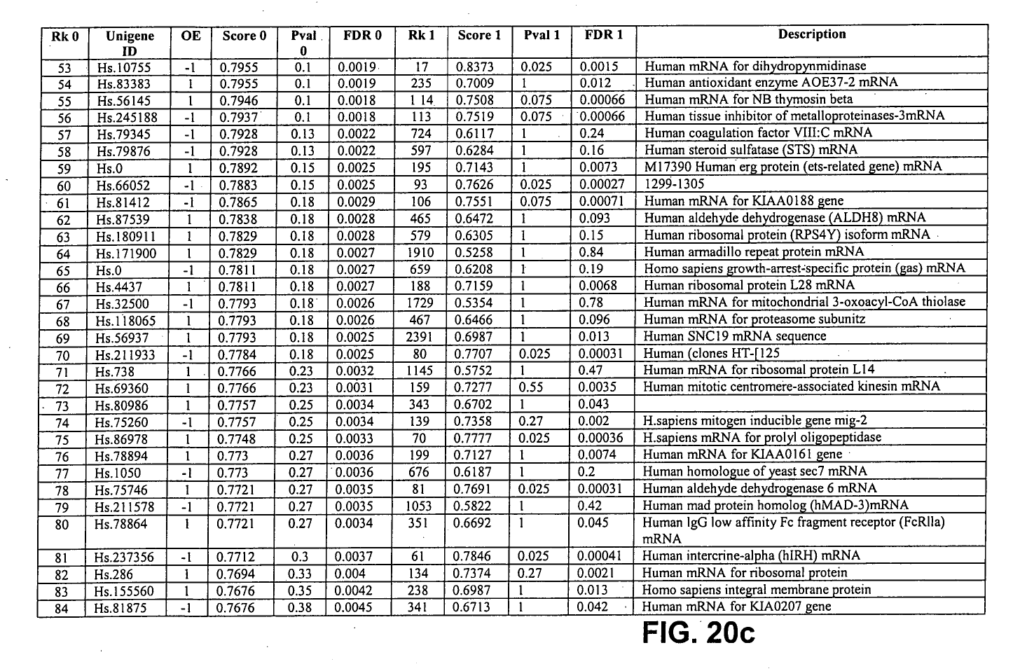

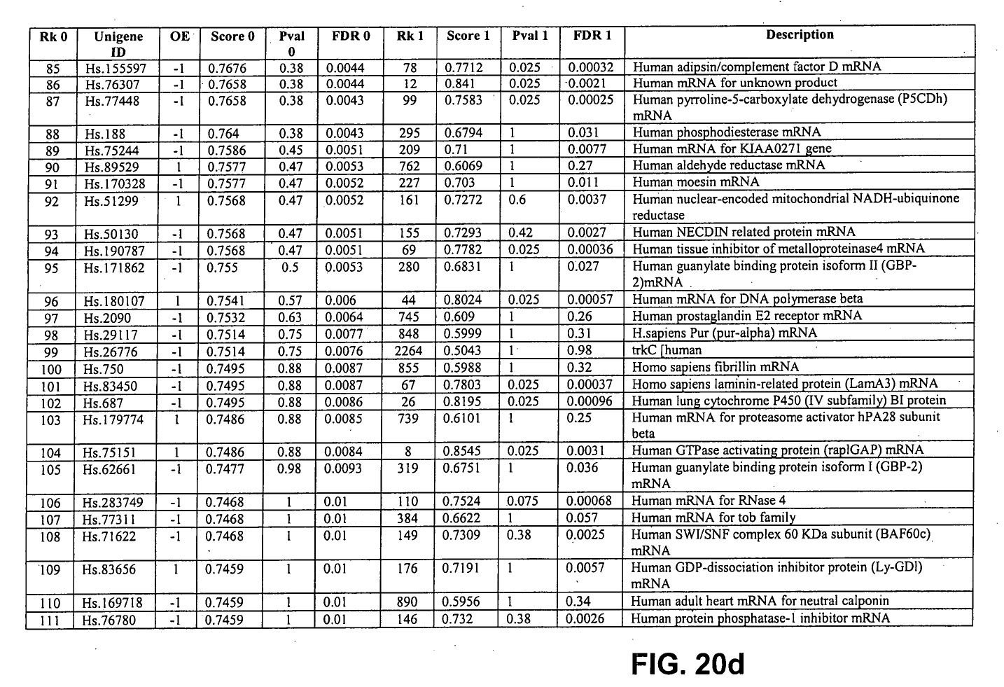

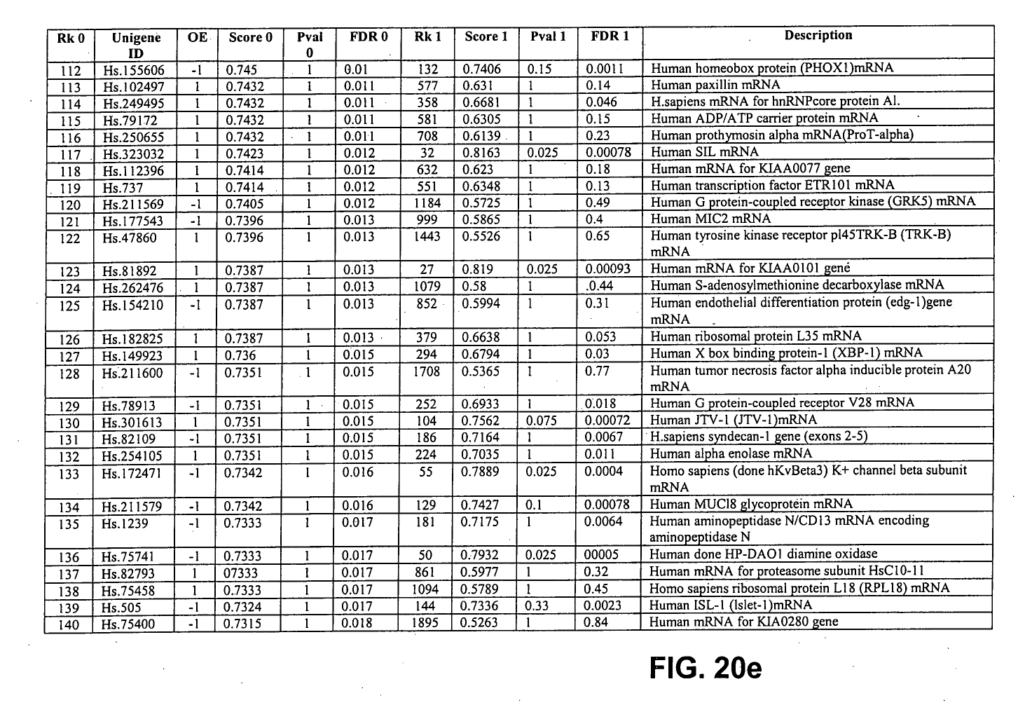

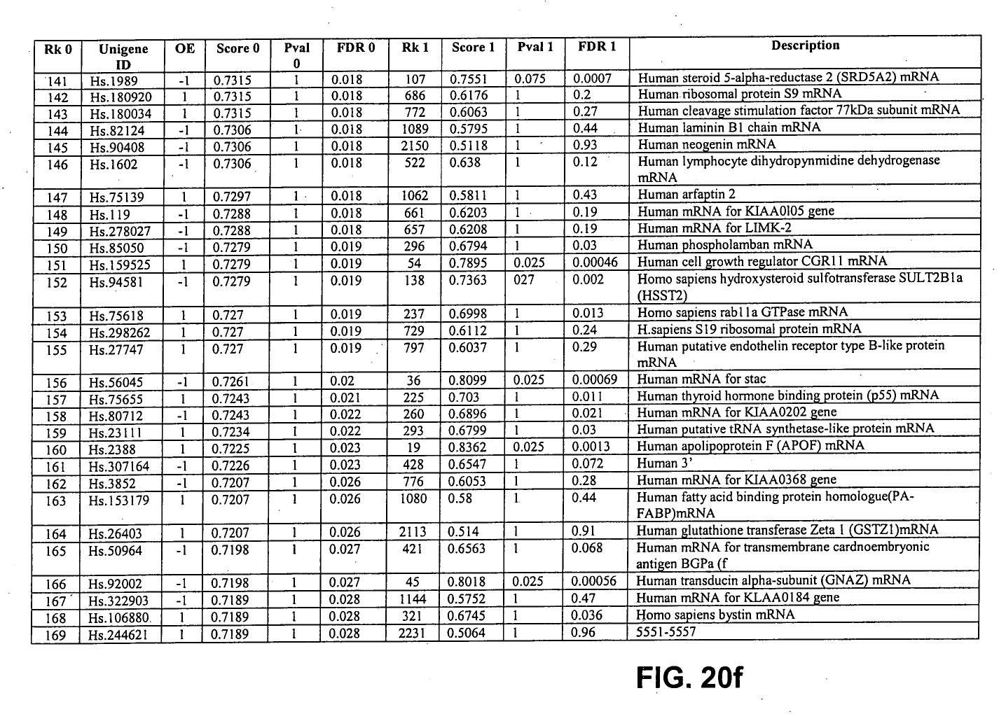

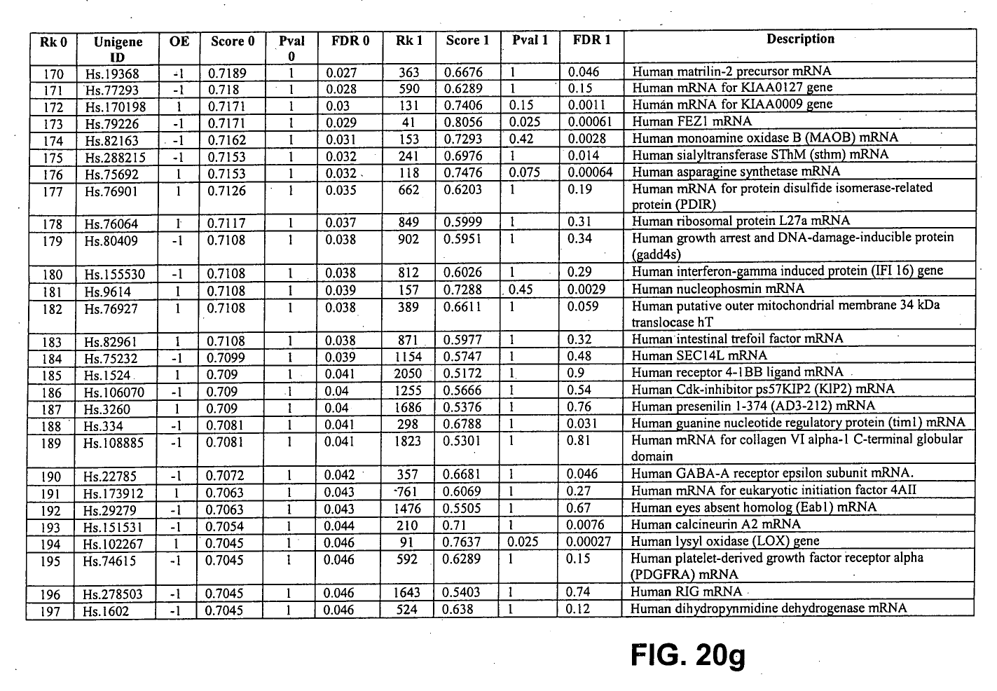

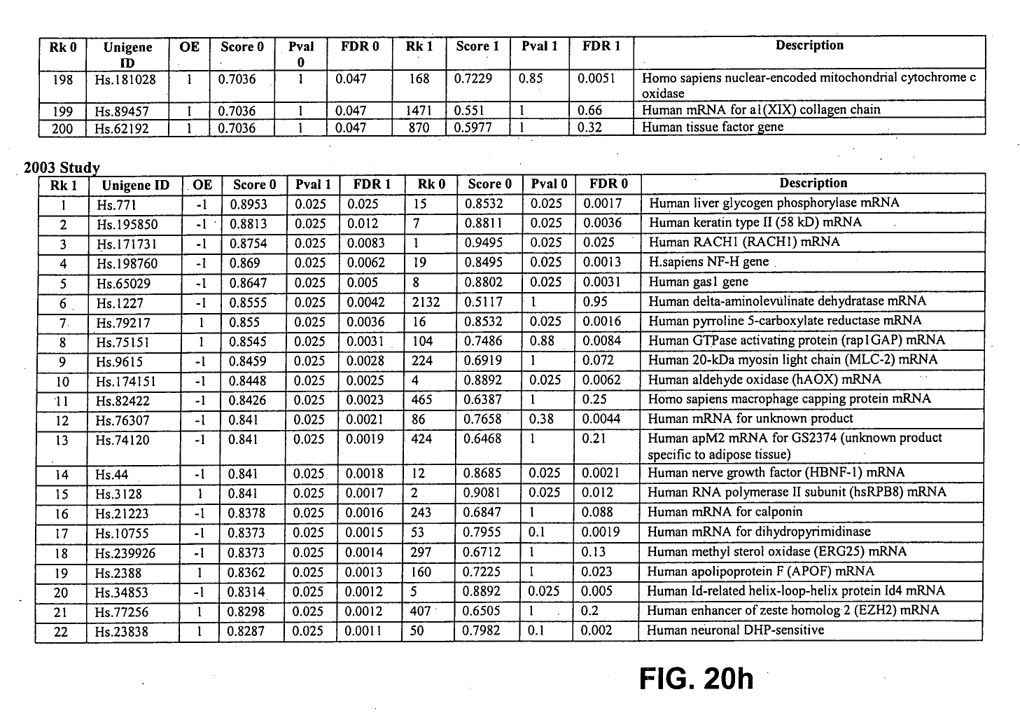

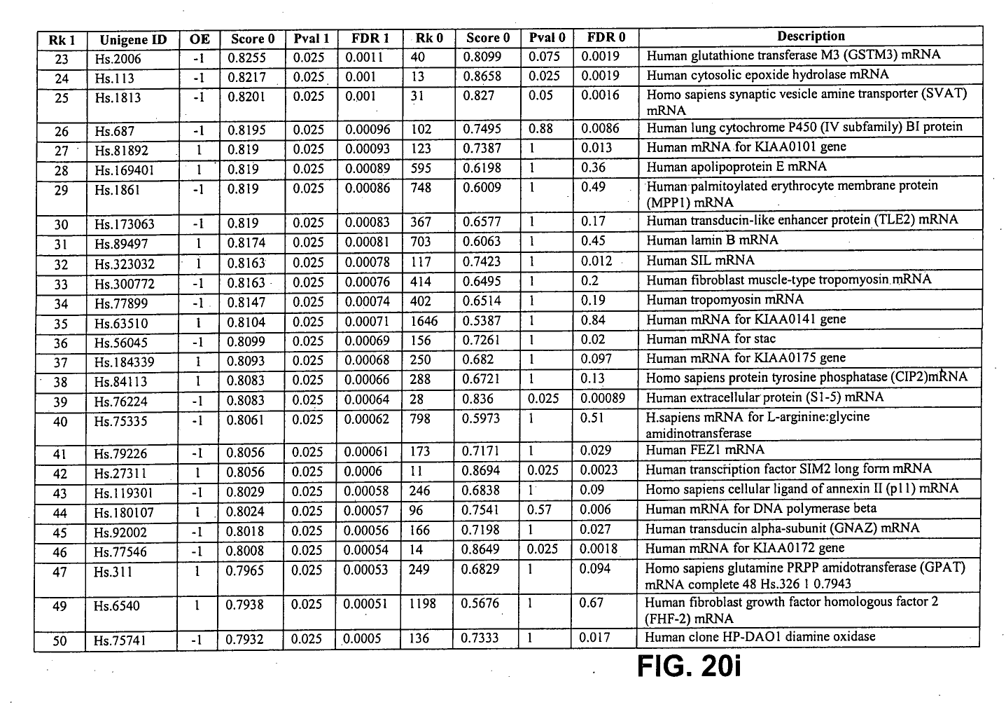

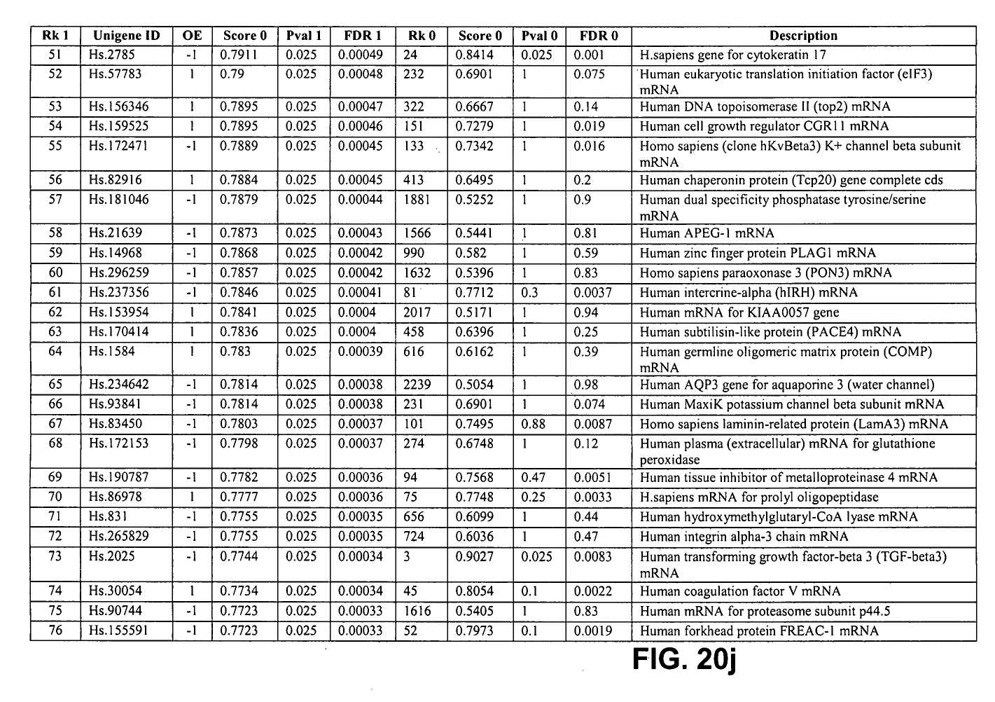

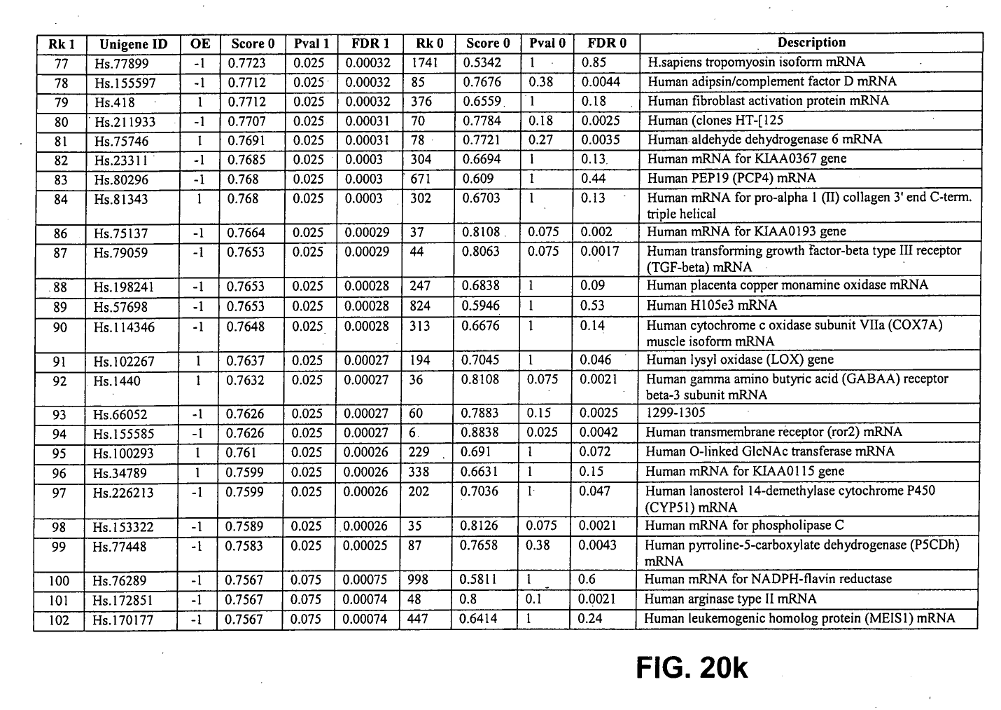

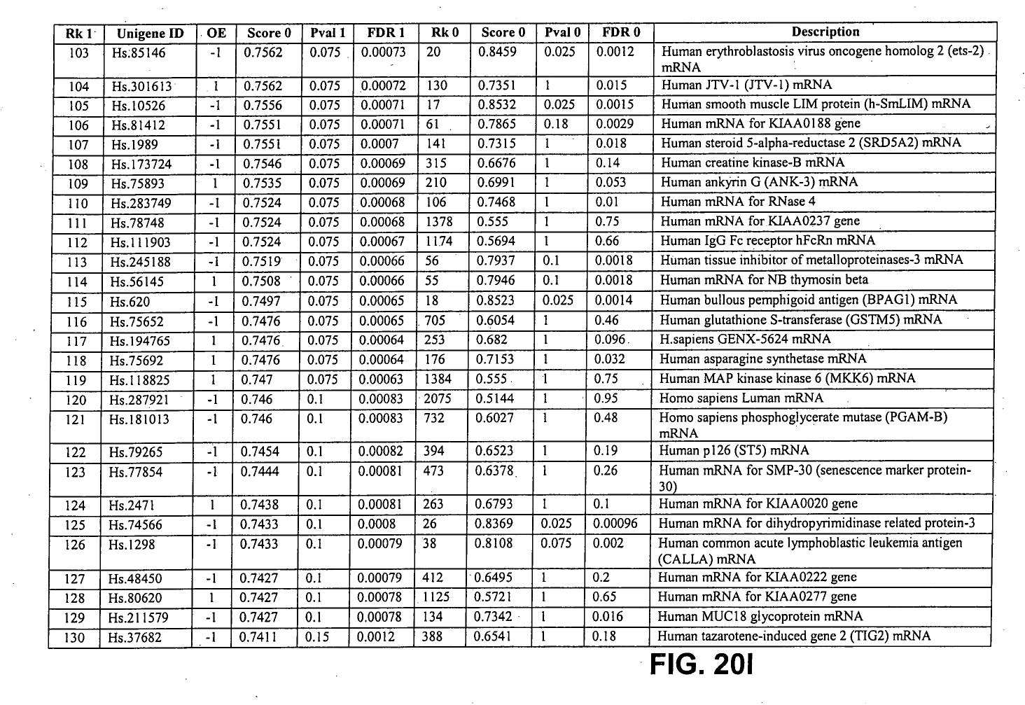

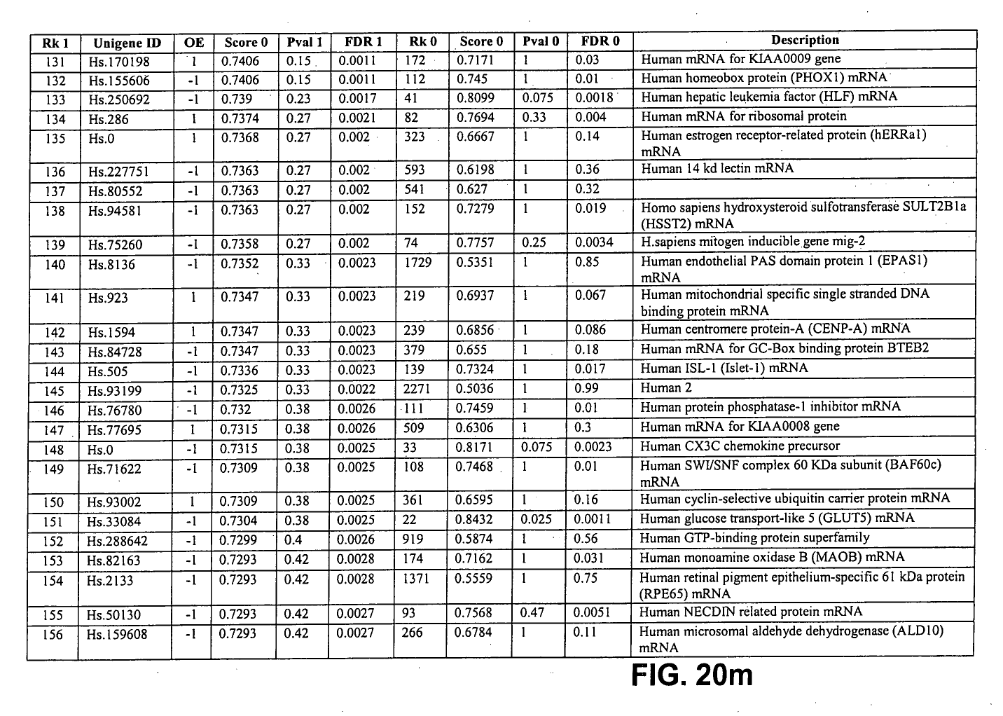

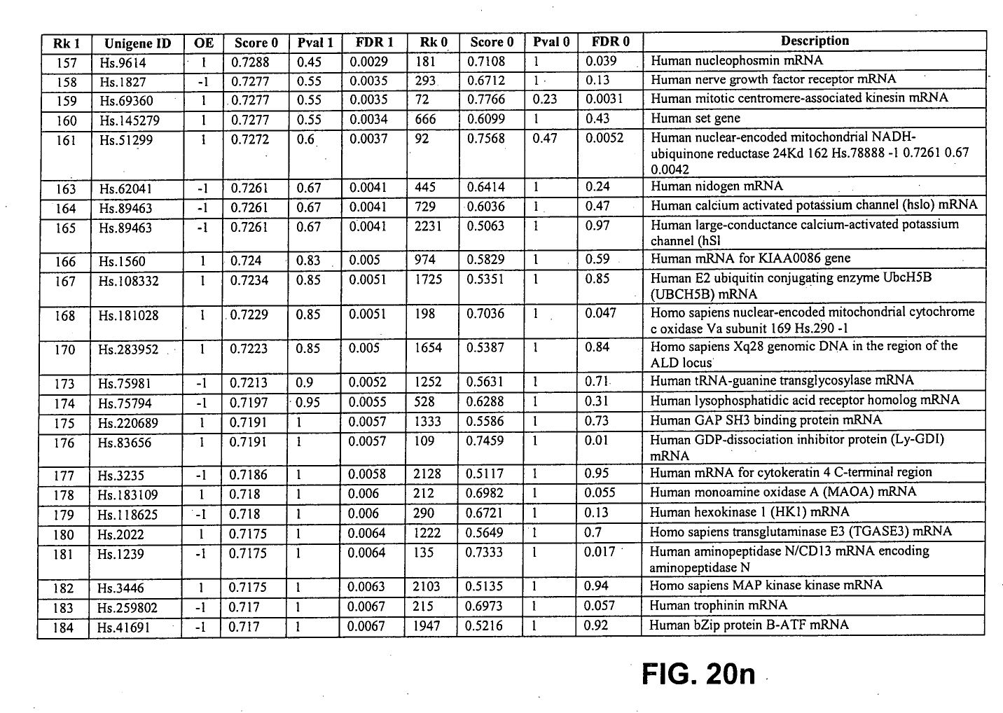

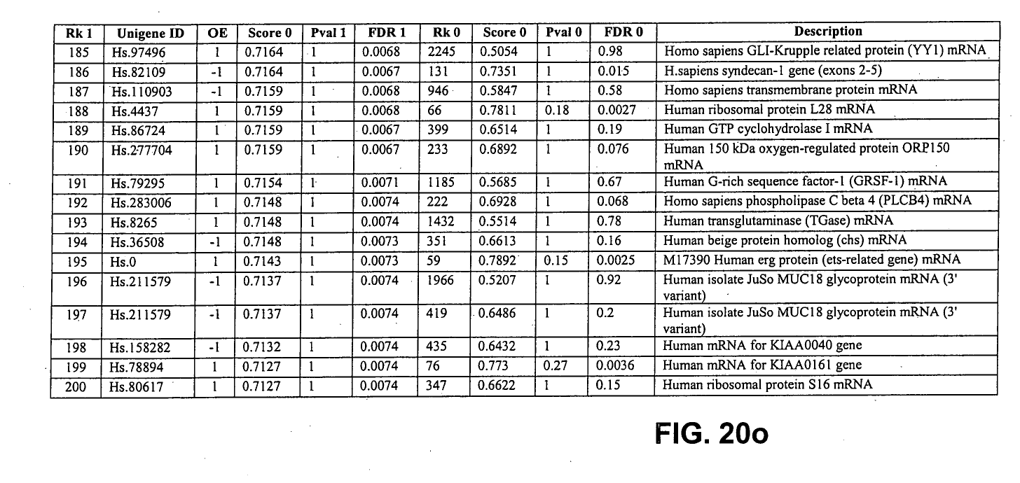

Separation of all tumor samples (G3 and G4) from all others was performed, with the G3 and G4 samples being grouped into the positive class and all samples grouped into the negative class. The top 200 genes in each study of Tumor G3/4 vs. others are listed in the tables in FIG. 20 for the 2001 study and the 2003 study. The genes were ranked in two ways, using the data of the first study (2001) and using the data of the second study (2003)

Most genes ranking high in one study also rank high in the other, with some notable exceptions. These exceptions may correspond to probes that do not match in both arrays even though their gene identification and descriptions match. They may also correspond to probes that “failed” to work in one array.

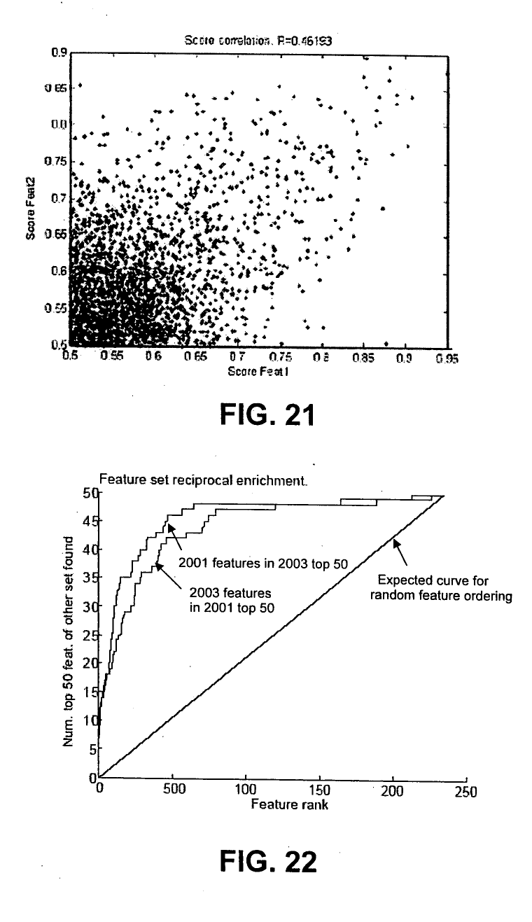

FIG. 21 illustrates how the AUC scores of the genes correlate in both studies for tumor versus all others. Looking at the upper right corner of the plot, most genes having a high score in one study also have a high score in the other. The correlation is significant, but not outstanding. The outliers have a good score in one study and a very poor score in the other. FIG. 22, a graph of reciprocal enrichment, shows that the genes extracted by one study are found by the other study much better than merely by chance. To create this graph, a set S of the top 50 ranking genes in one study was selected. Then, varying the number of top ranking genes selected from the other study, the number of genes from set S was determined. If the ranking obtained by the other study were truly random, the genes of S should be uniformly distributed and the progression of the number of genes of S found as a function of the size of the gene set would be linear. Instead, most genes of S are found in the top ranking genes of the other study.

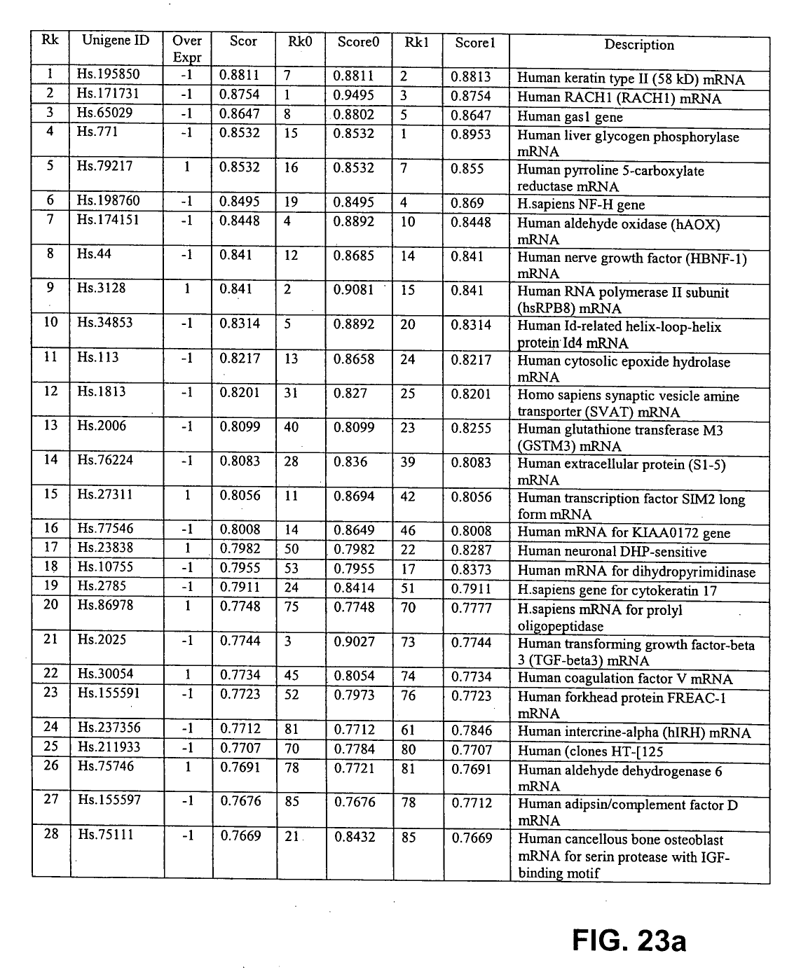

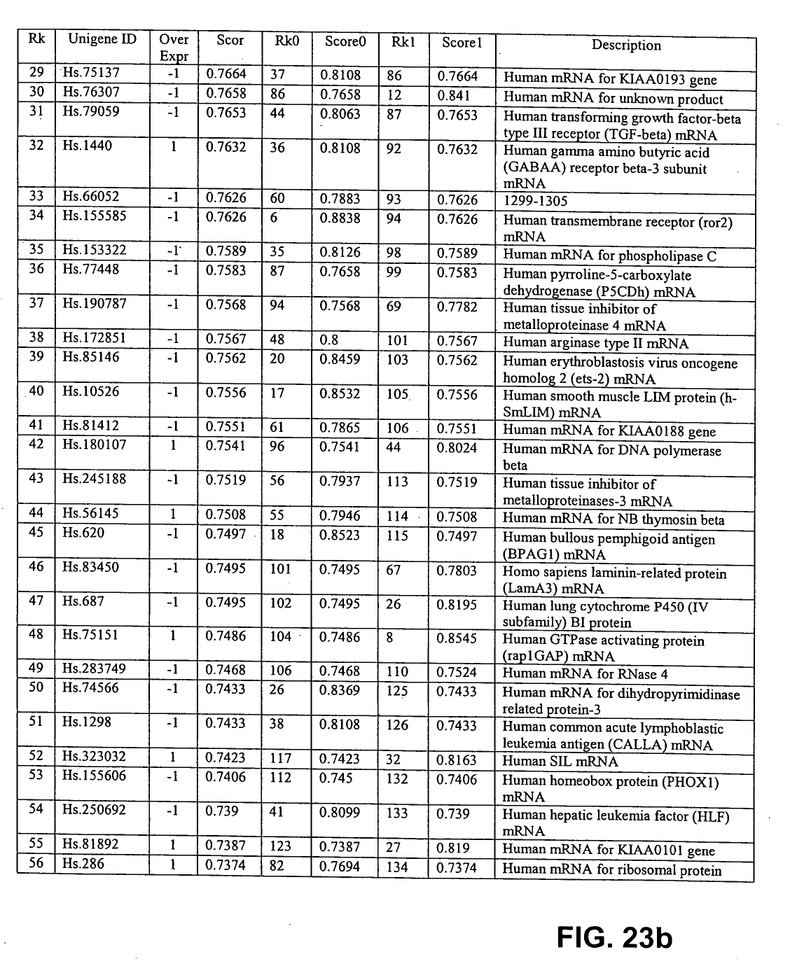

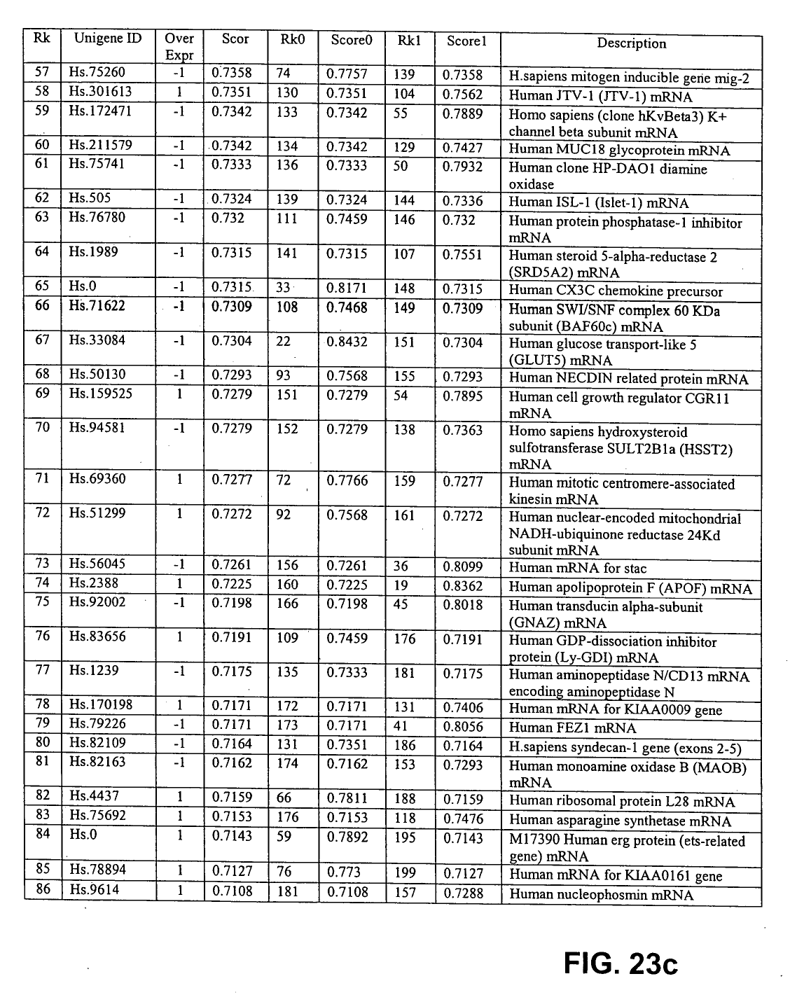

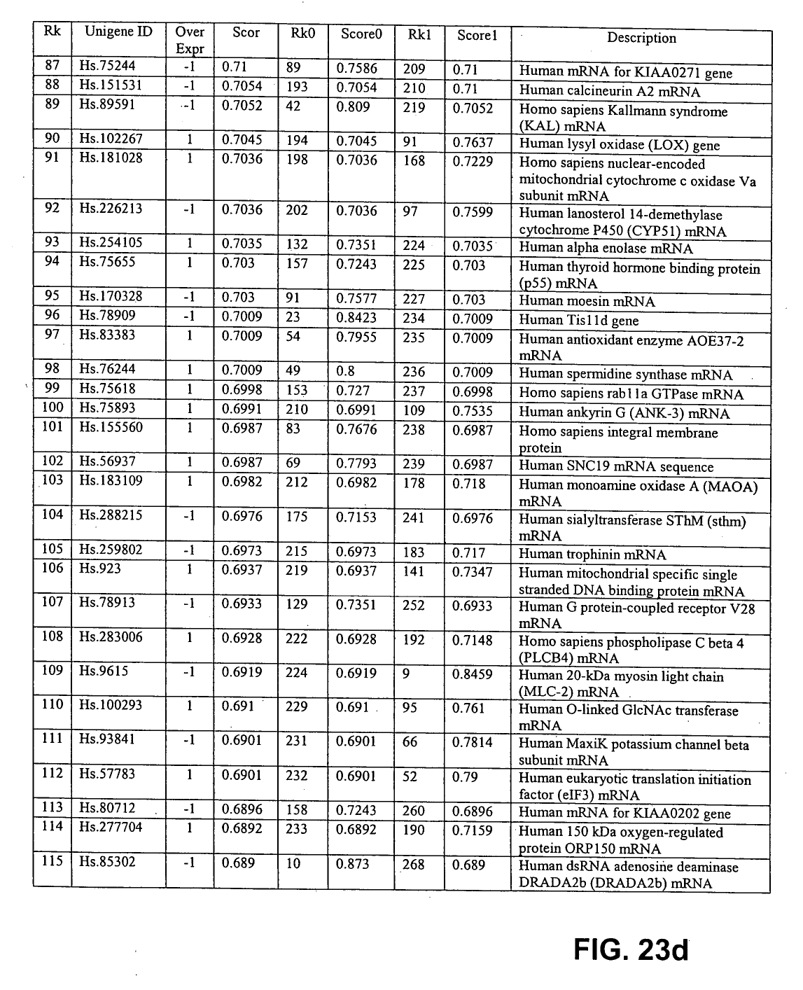

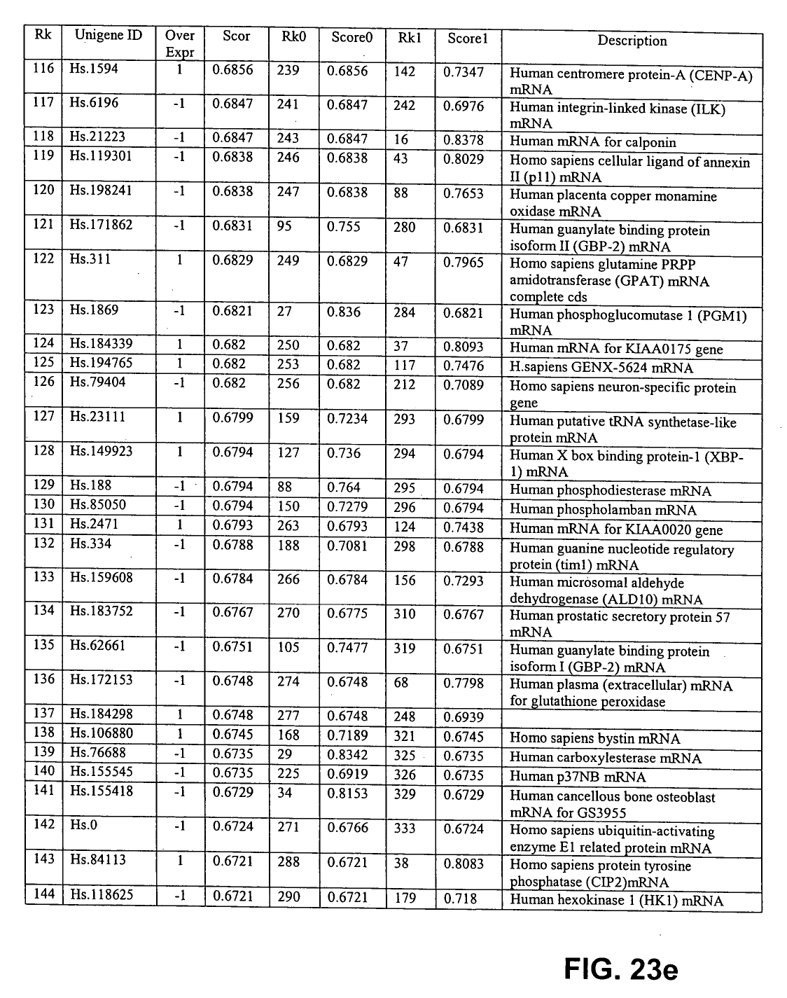

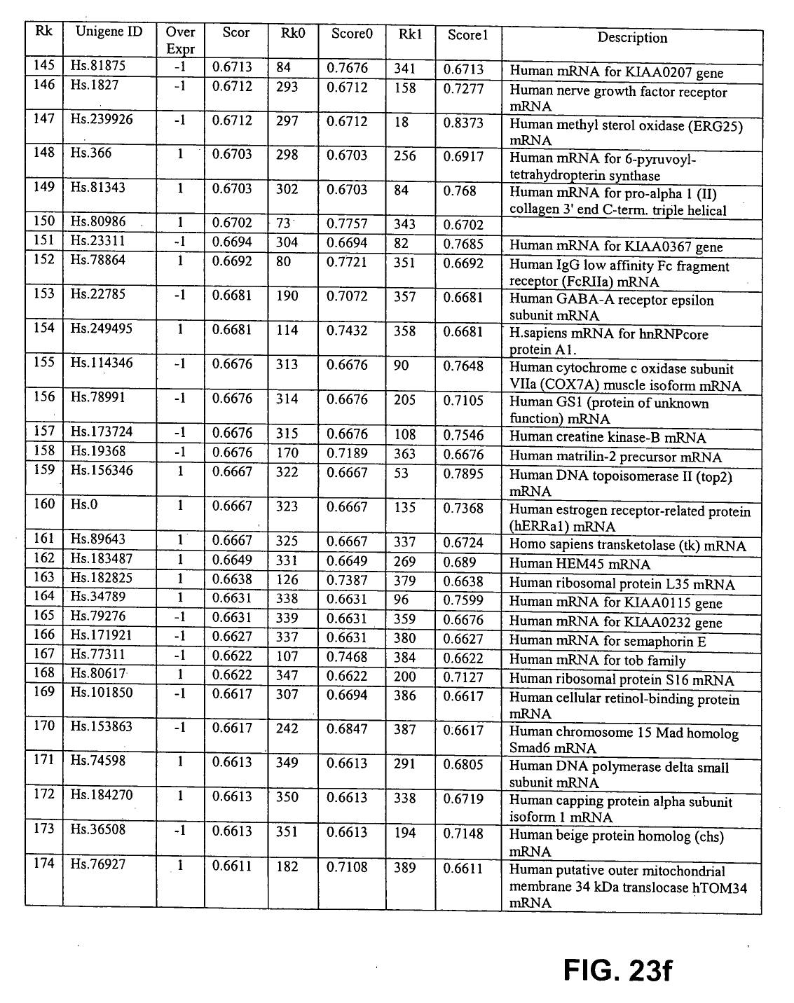

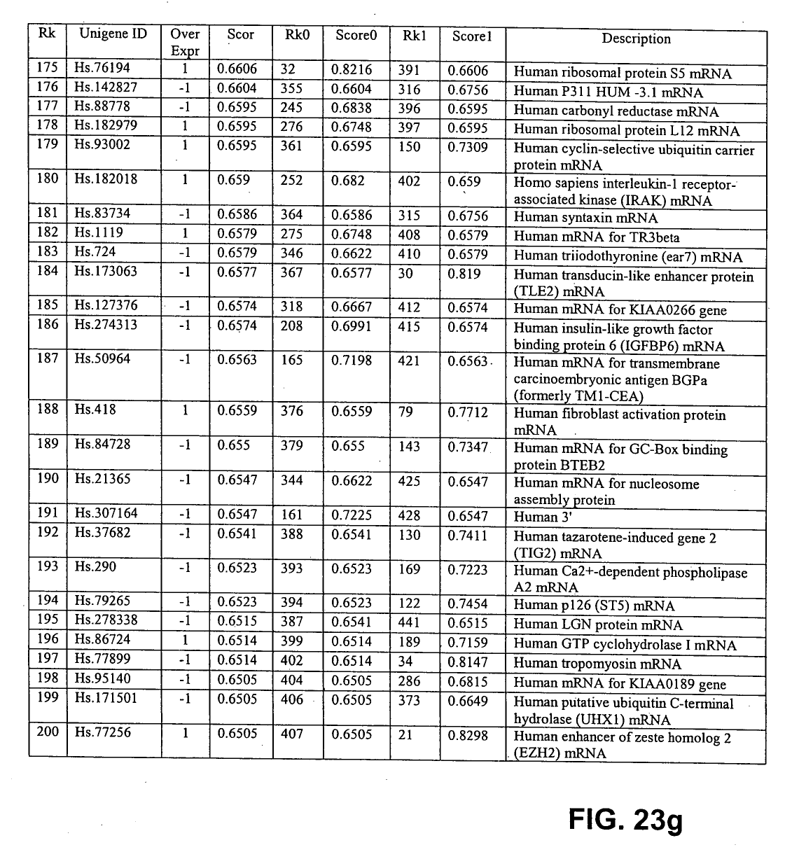

The table in FIG. 23 shows the top 200 genes resulting from the feature ranking by consensus between the 2001 study and the 2003 study Tumor G3/4 vs. others. Ranking is performed according to a score that is the minimum of score 0 and score 1.

Training of the classifier was done with the data of one study while testing used the data of the other study. The results are similar for the three classifiers that were tried: SVM, linear ridge regression and Golub classifier. Approximately 90% accuracy can be achieved in both cases with about 10 features. Better “cheating” results are obtained with the consensus features. This serves to validate the consensus features, but the performances cannot be used to predict the accuracy of a classifier on new data. An SVM was trained using the two best features of the 2001 study and the sample of the 2001 study as the training data. The samples from the 2003 study were used as test data to achieve an error rate of 16% is achieved. The tumor and non-tumor samples are well separated, but that, in spite of normalization, the distributions of the samples is different between the two studies.

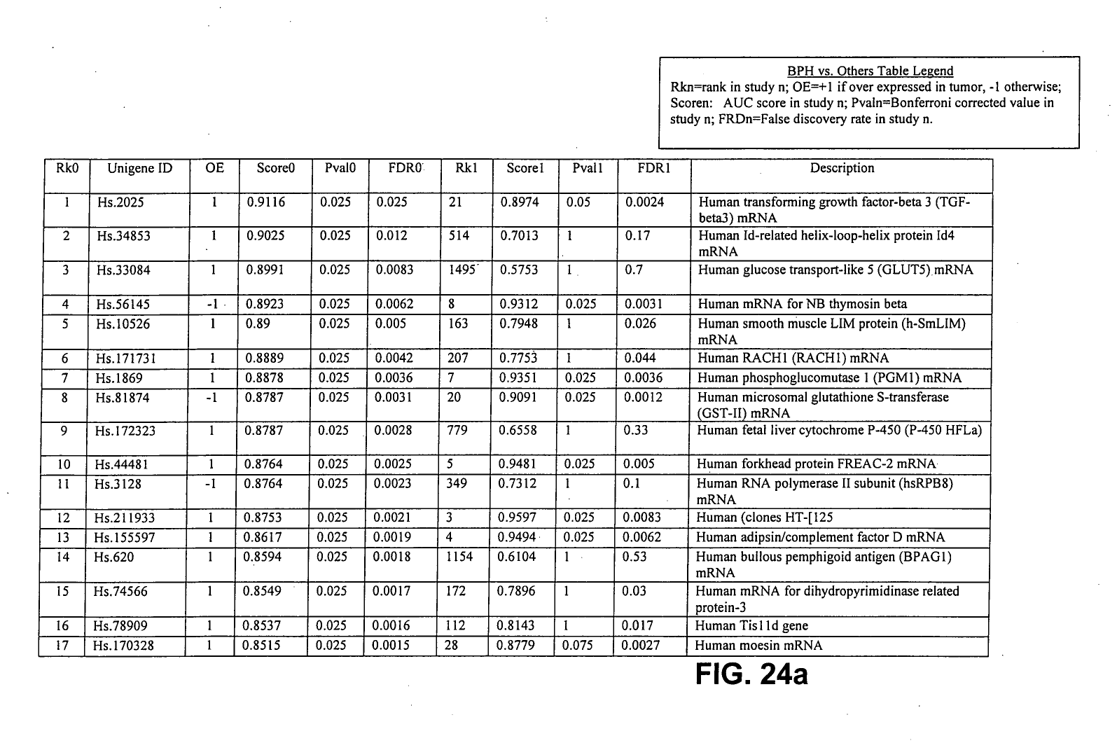

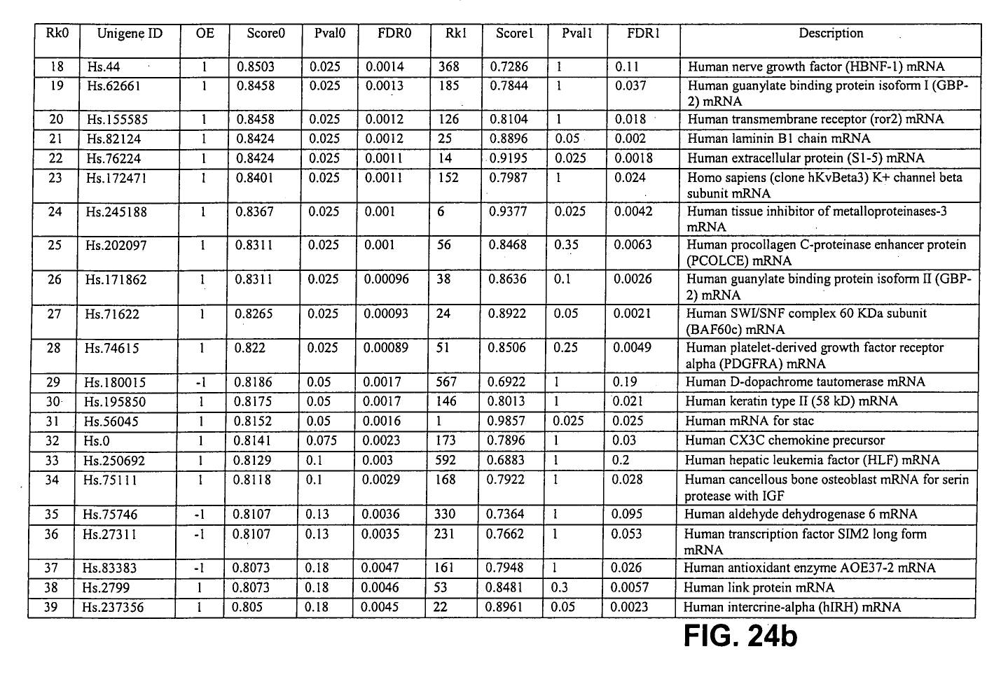

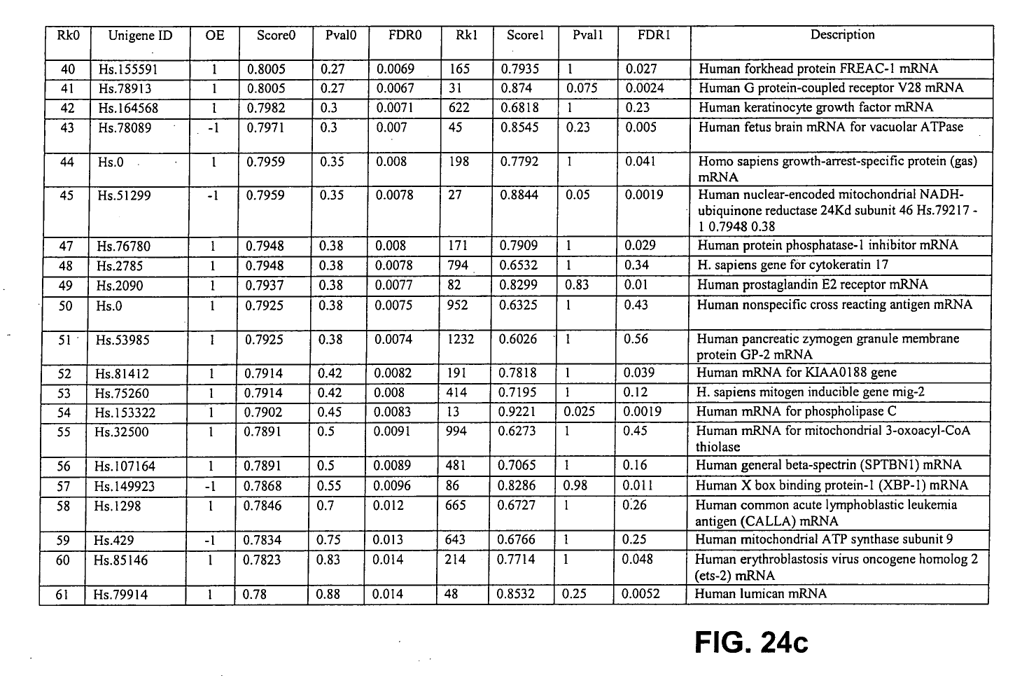

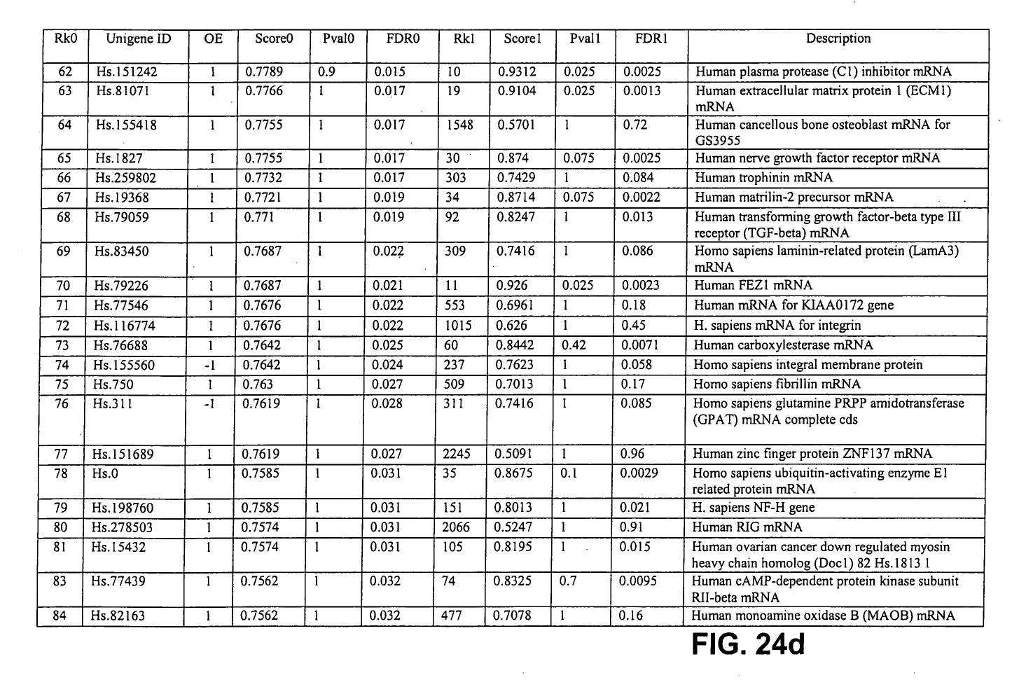

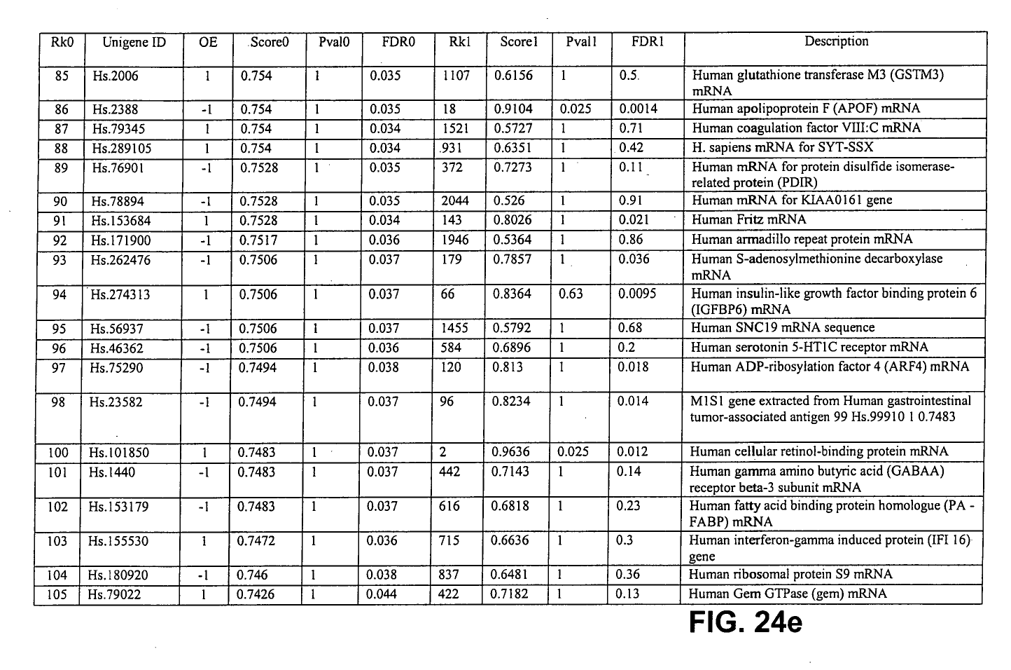

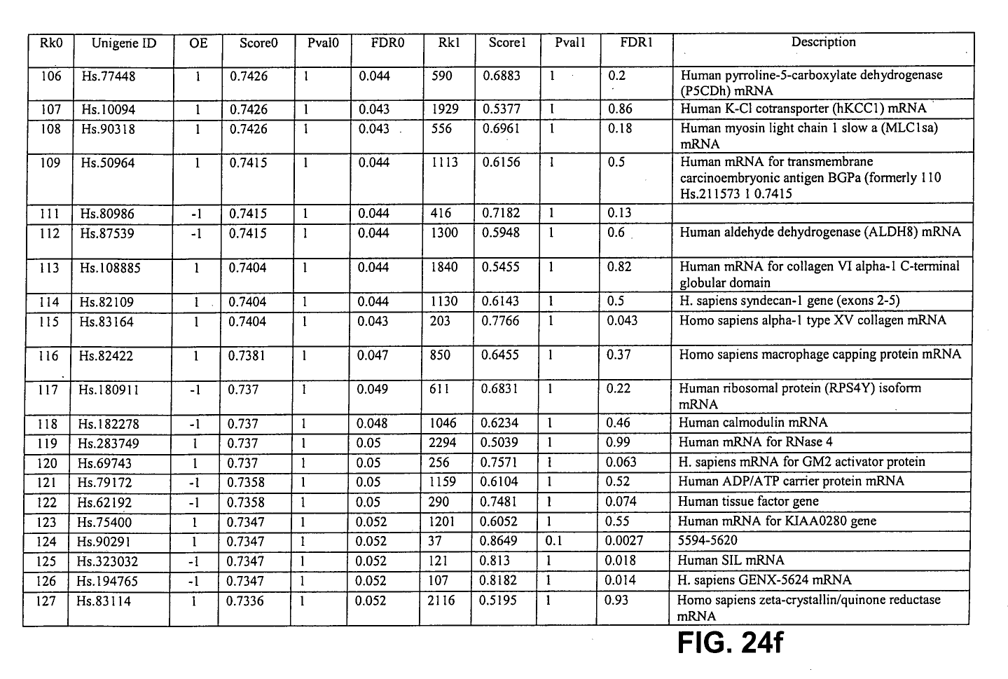

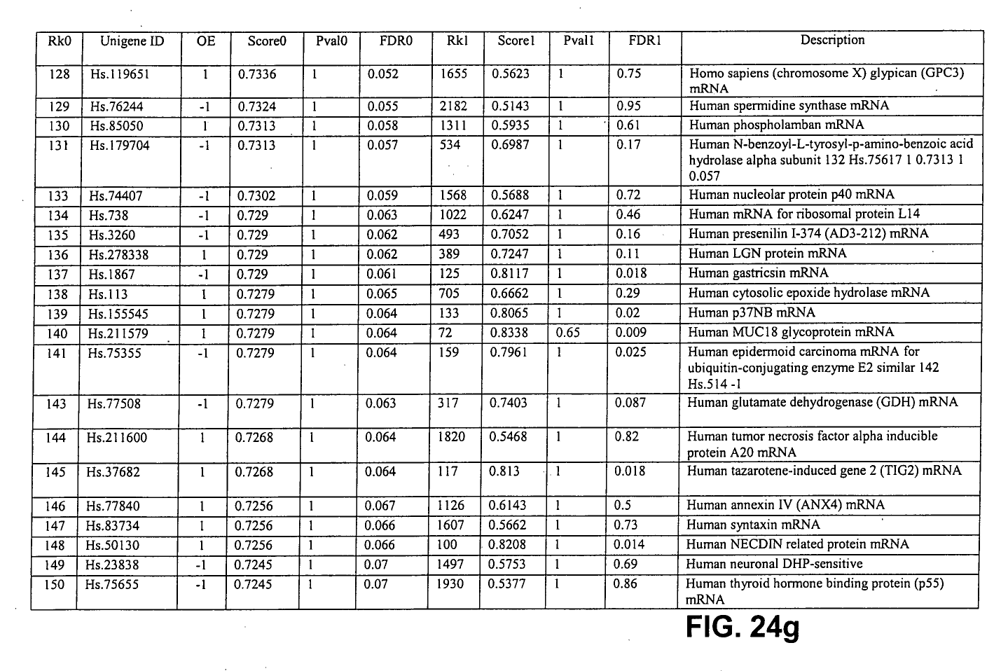

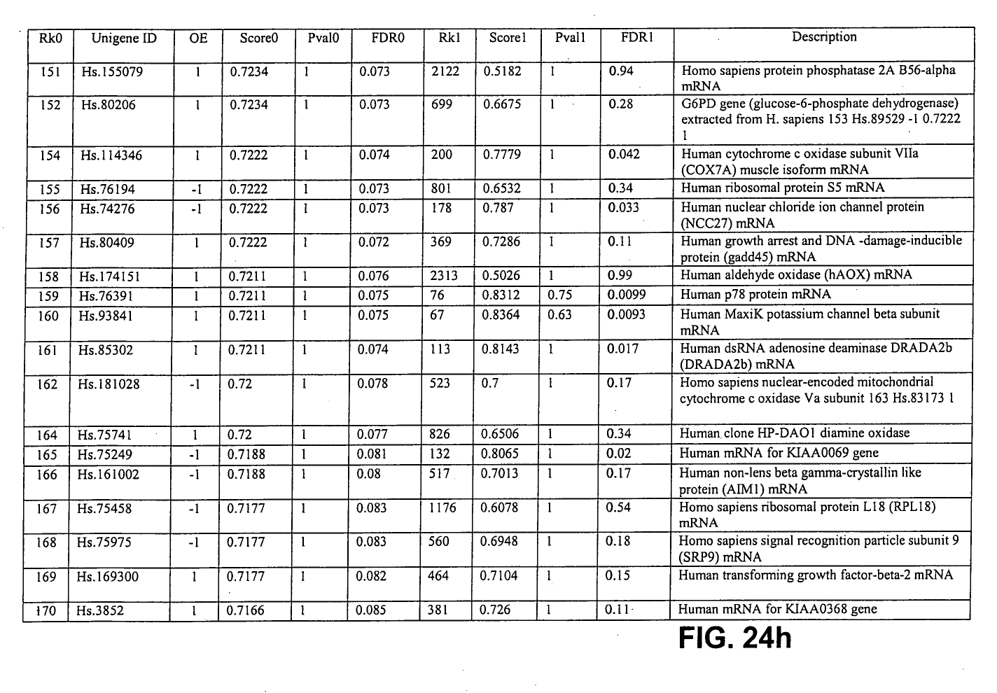

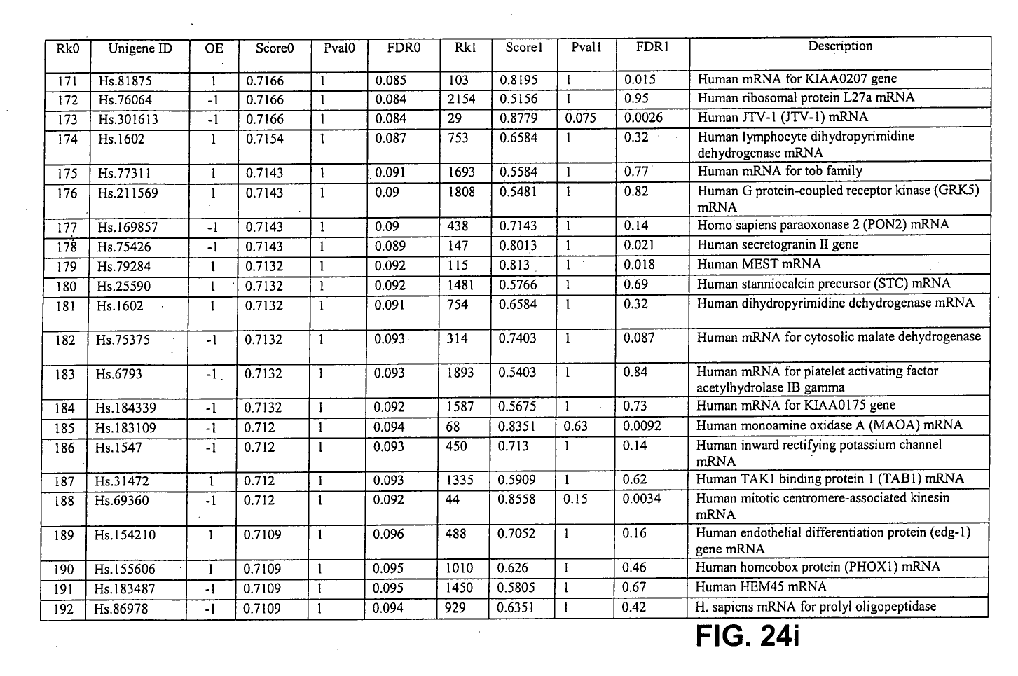

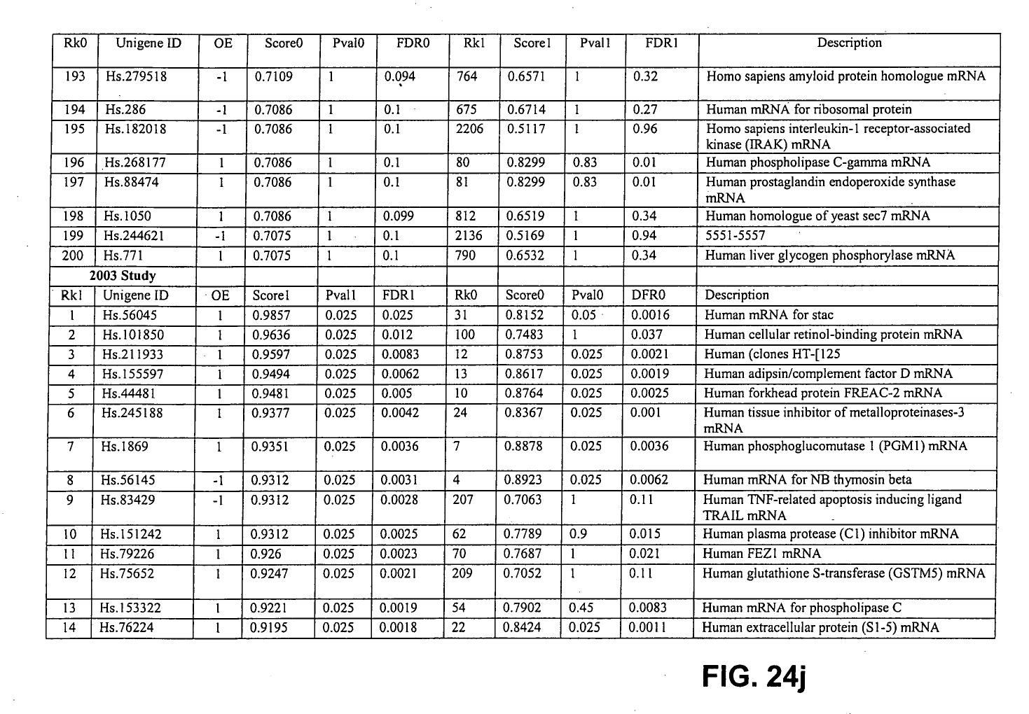

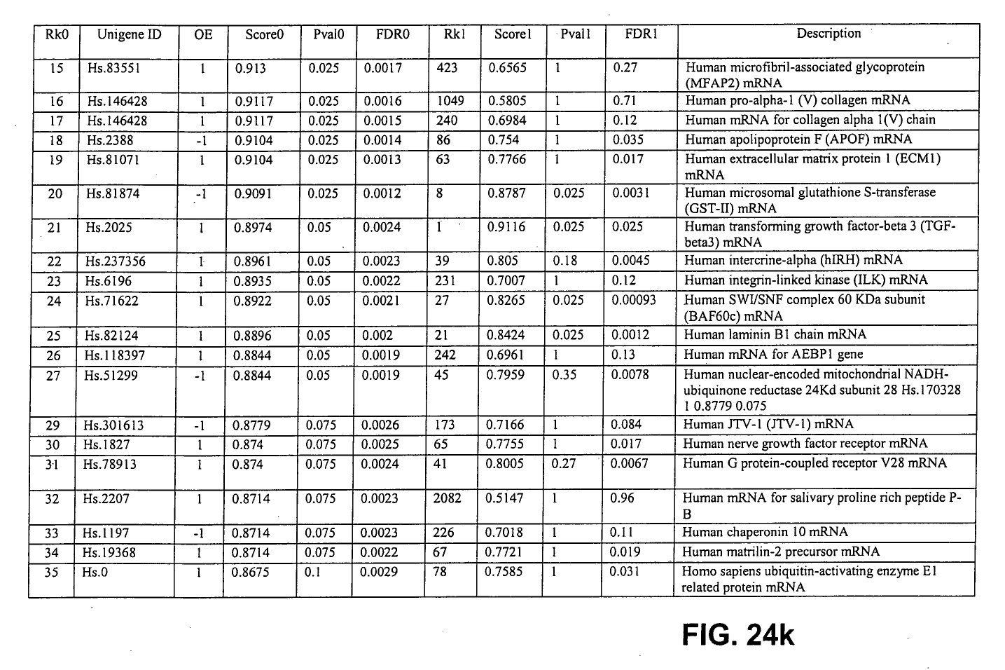

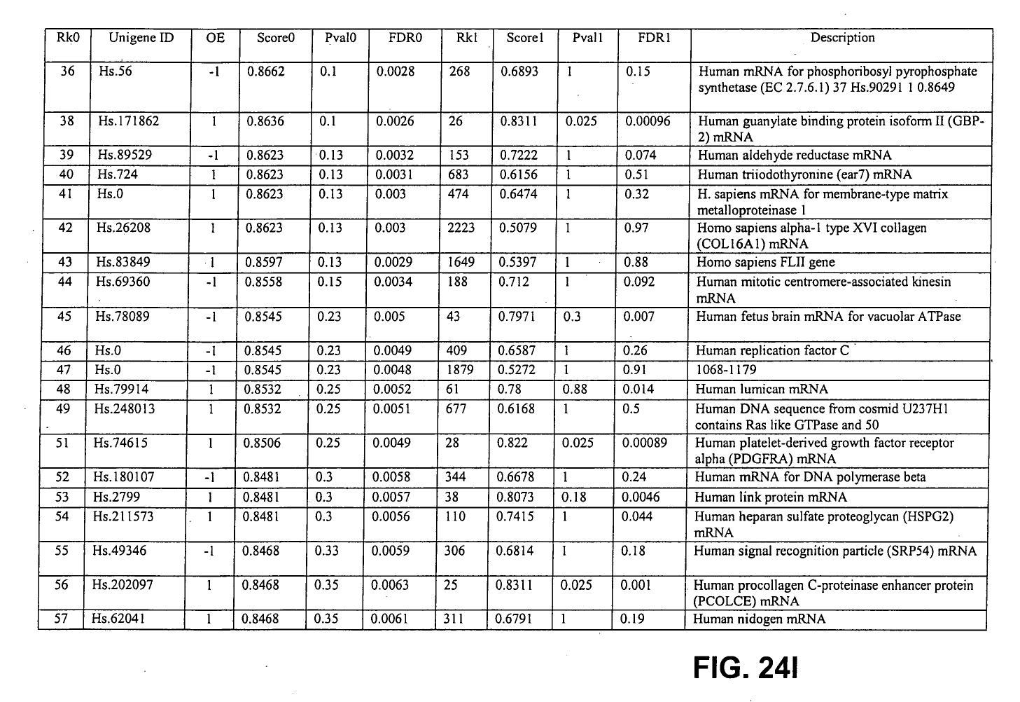

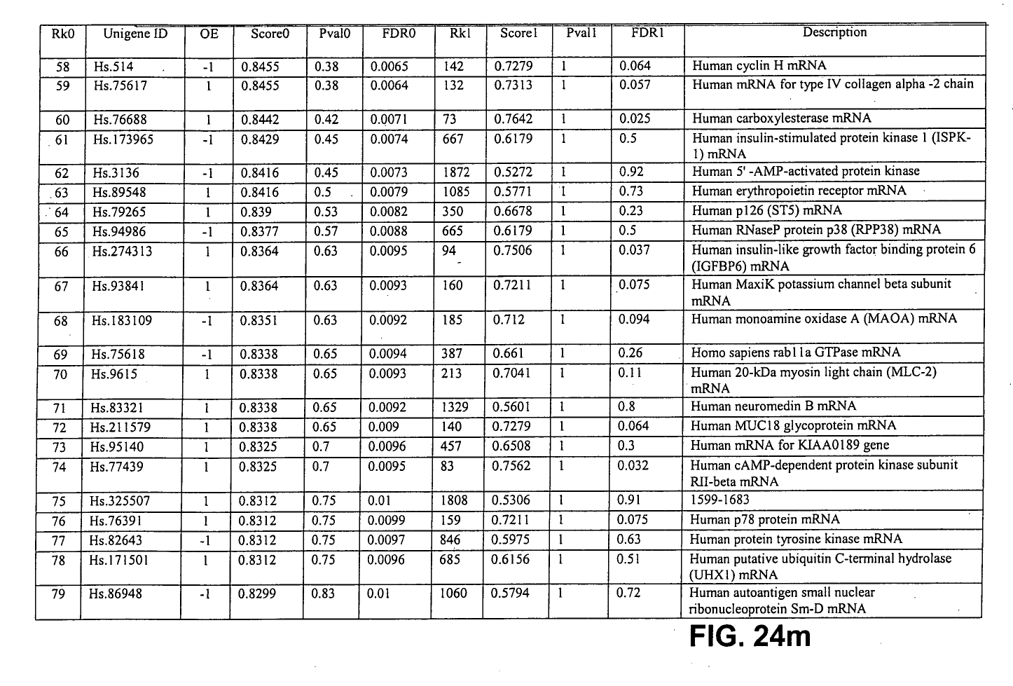

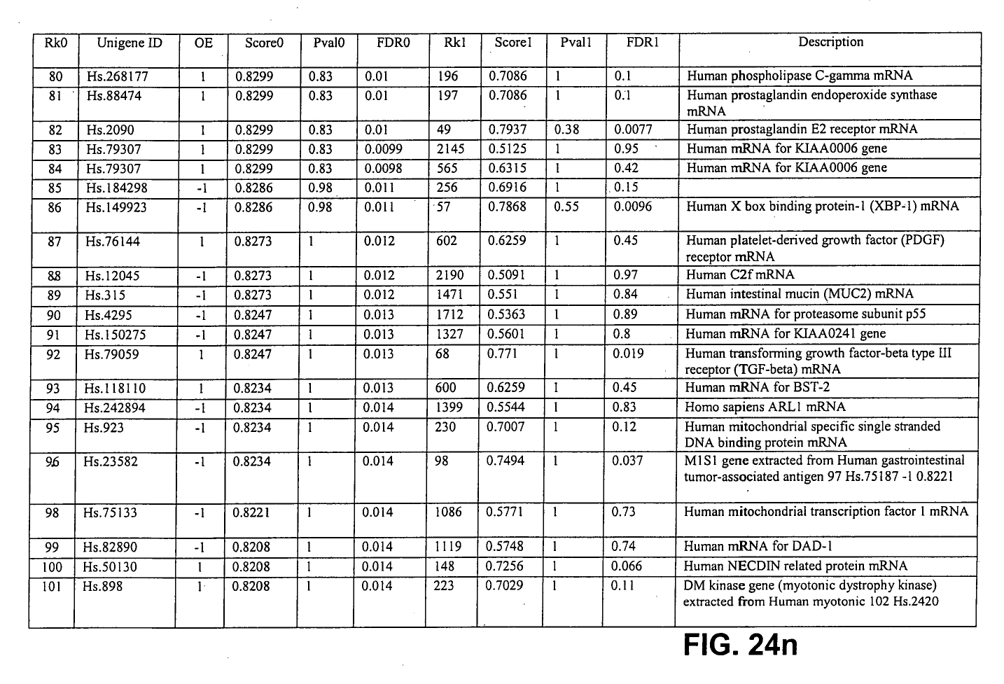

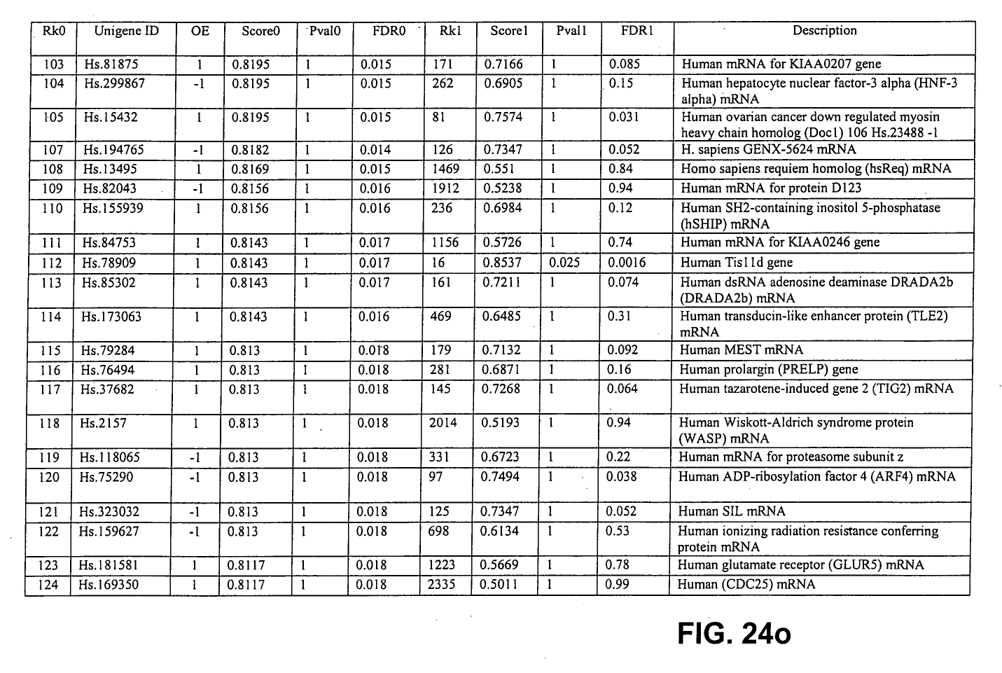

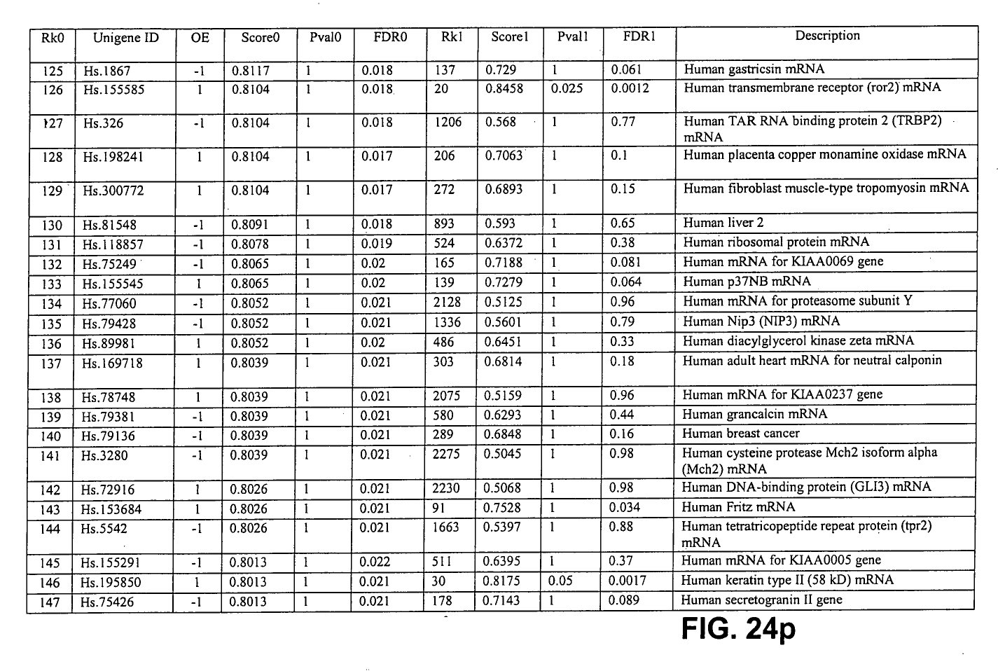

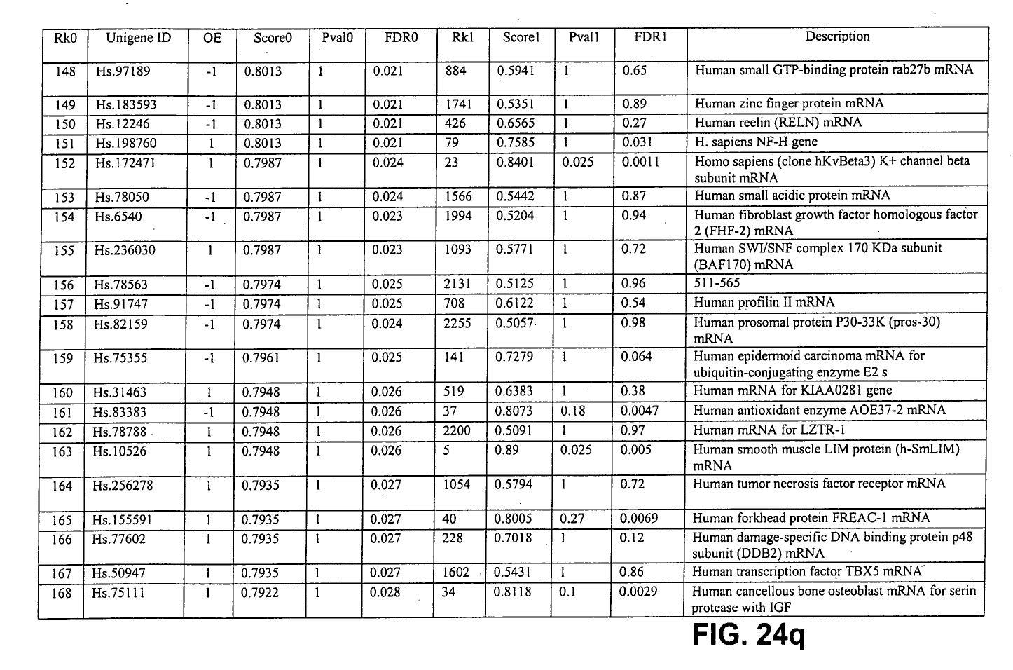

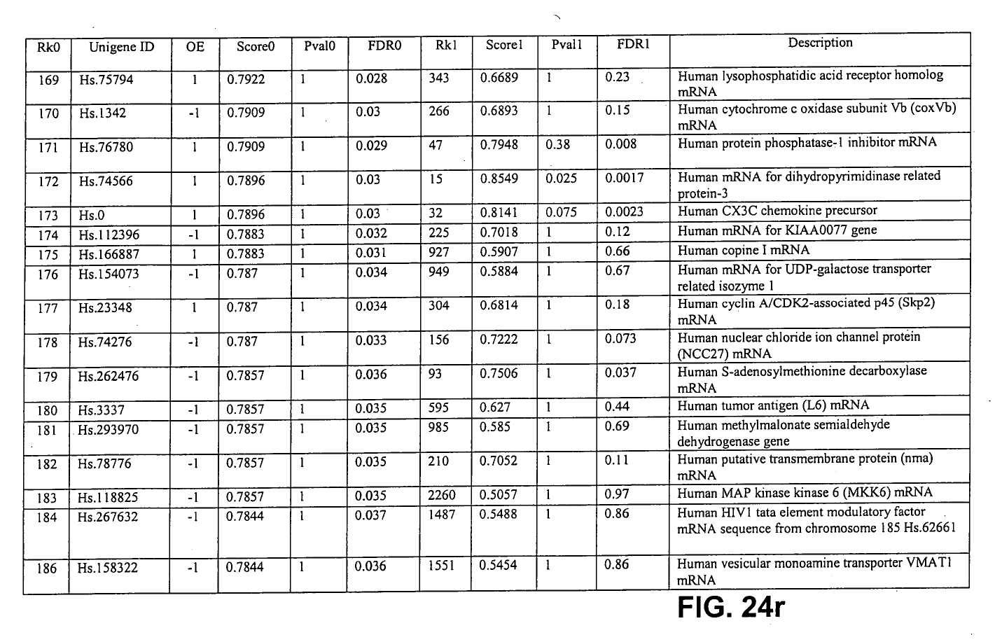

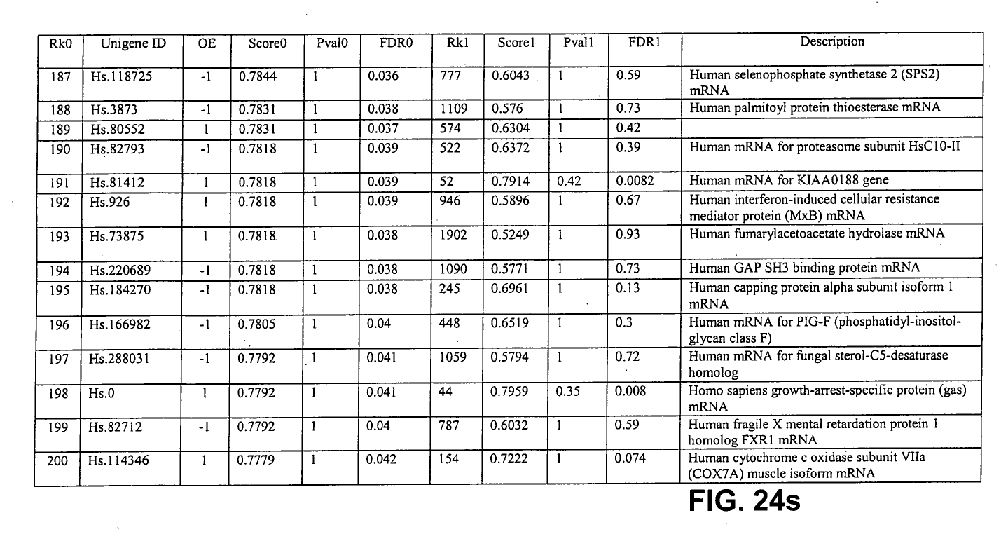

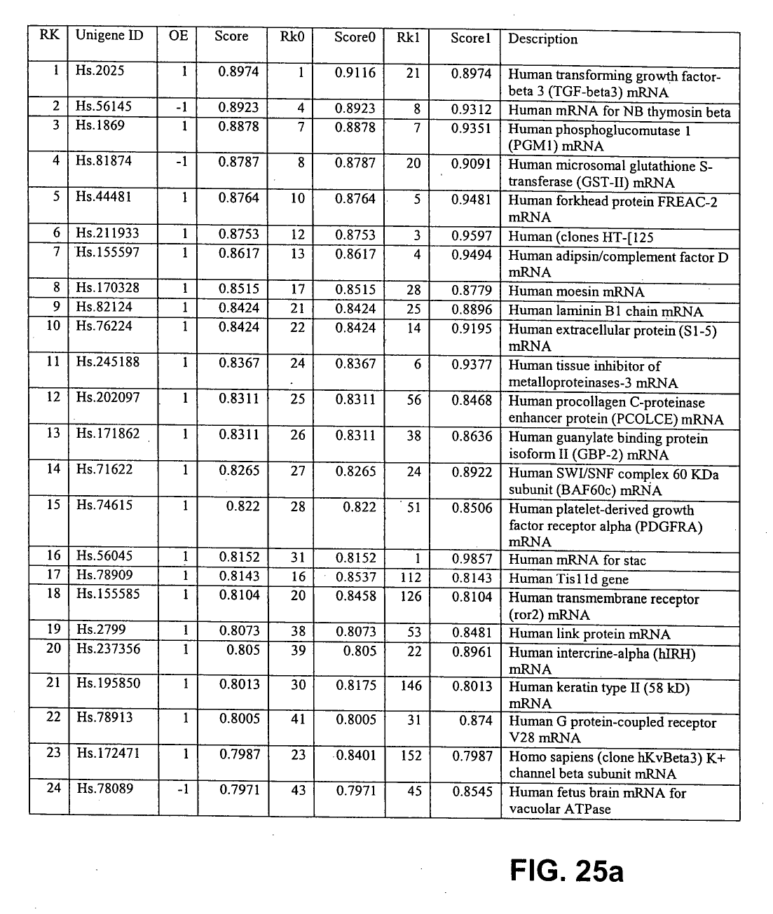

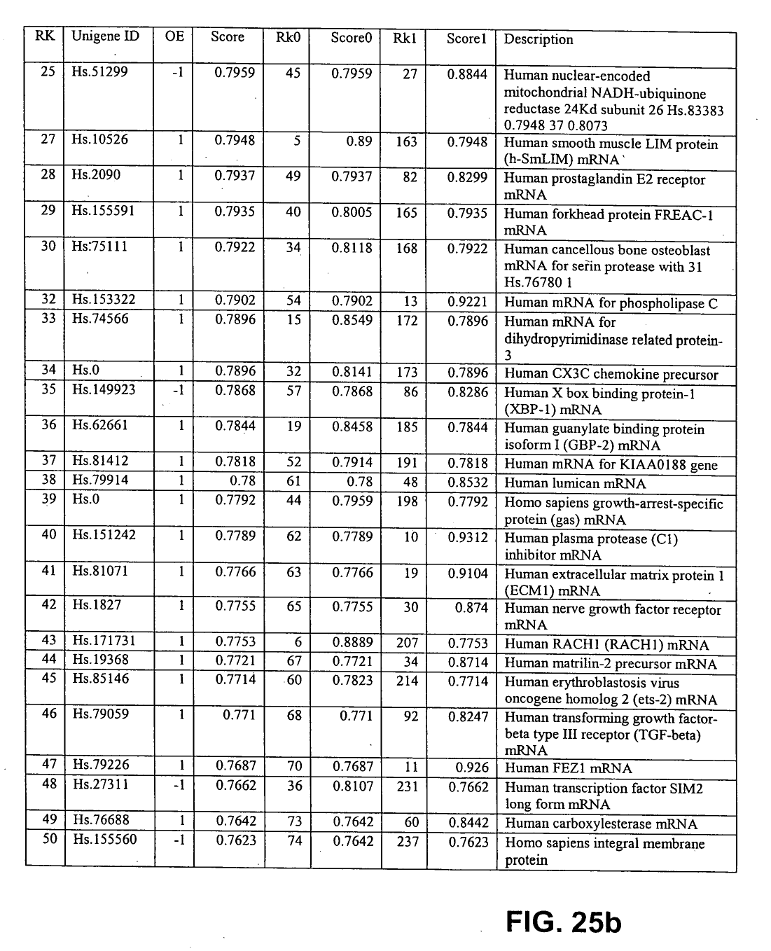

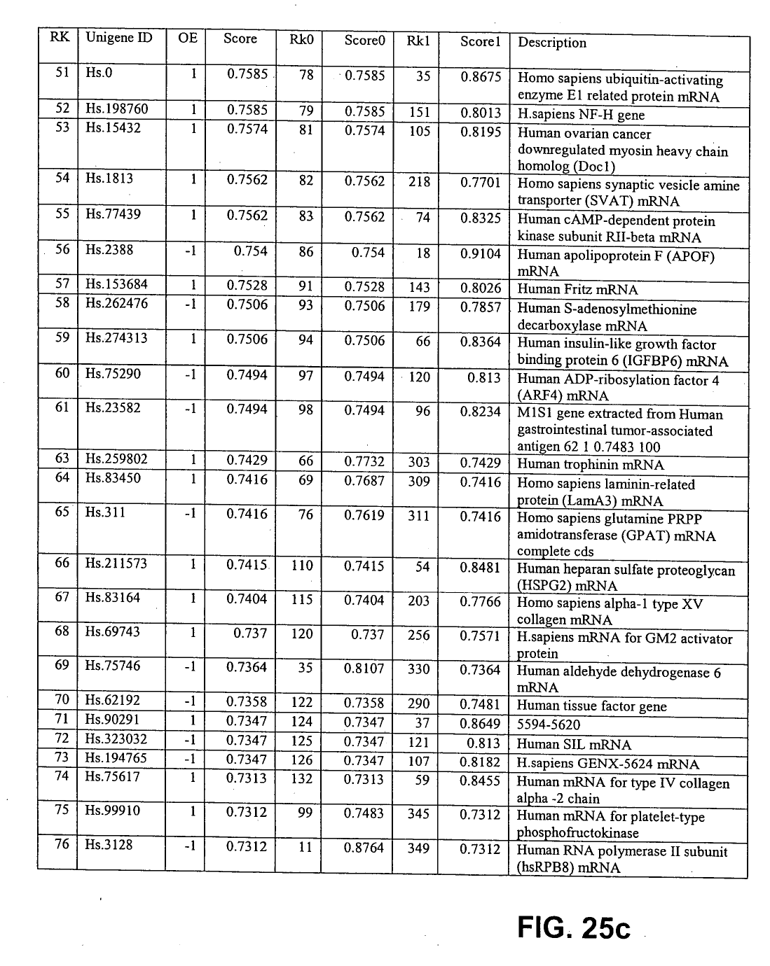

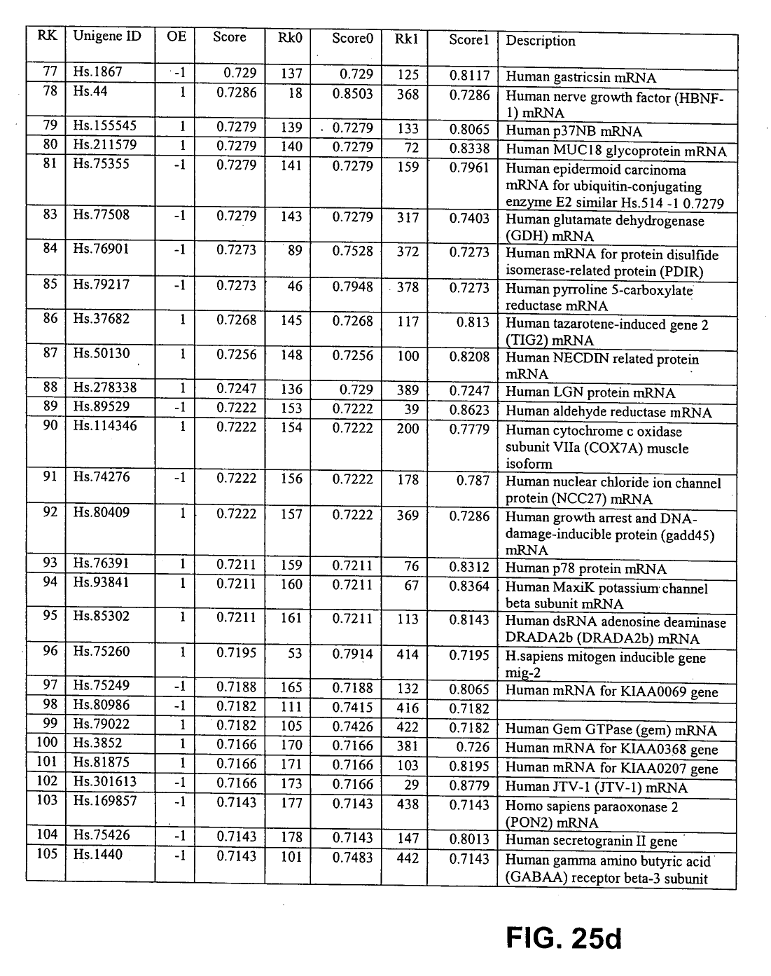

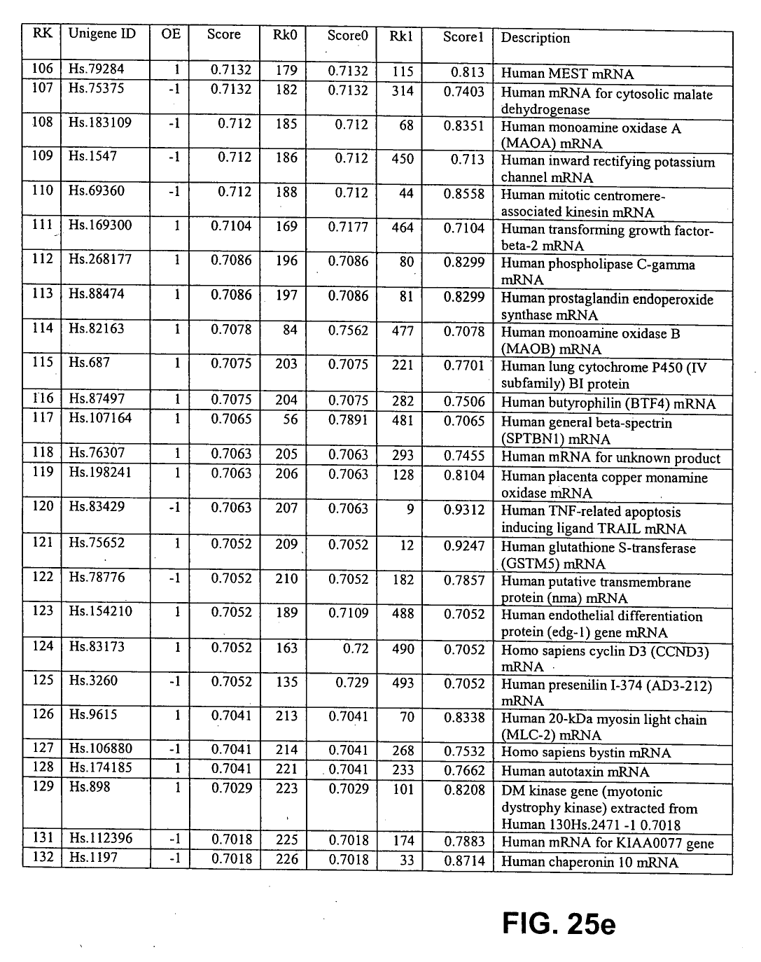

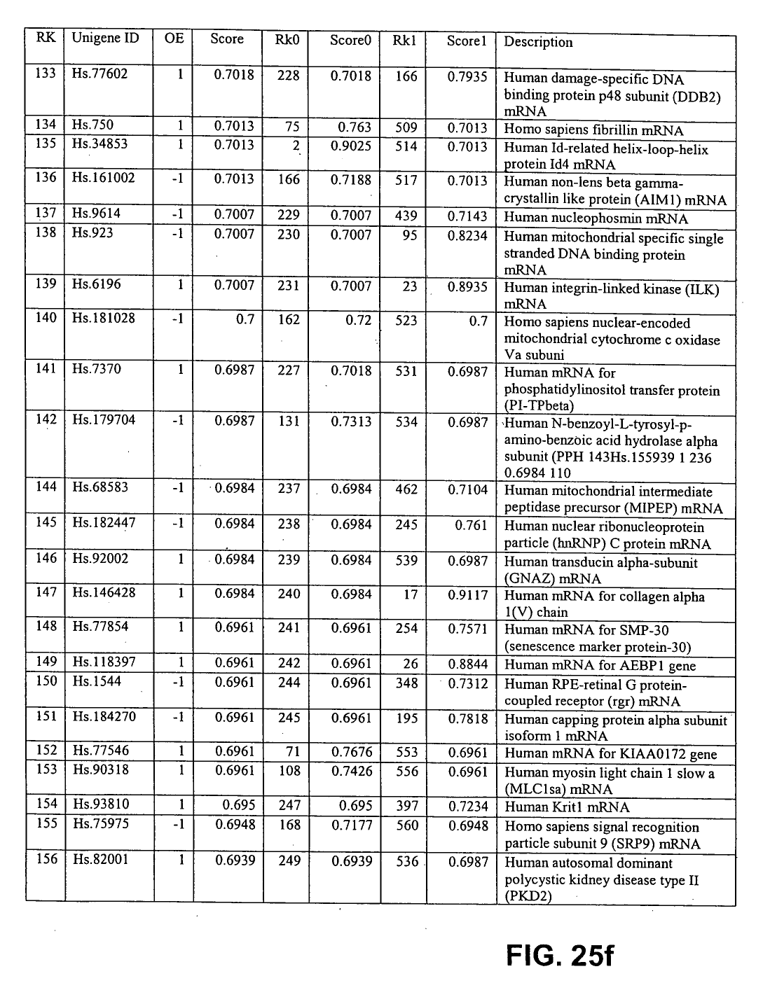

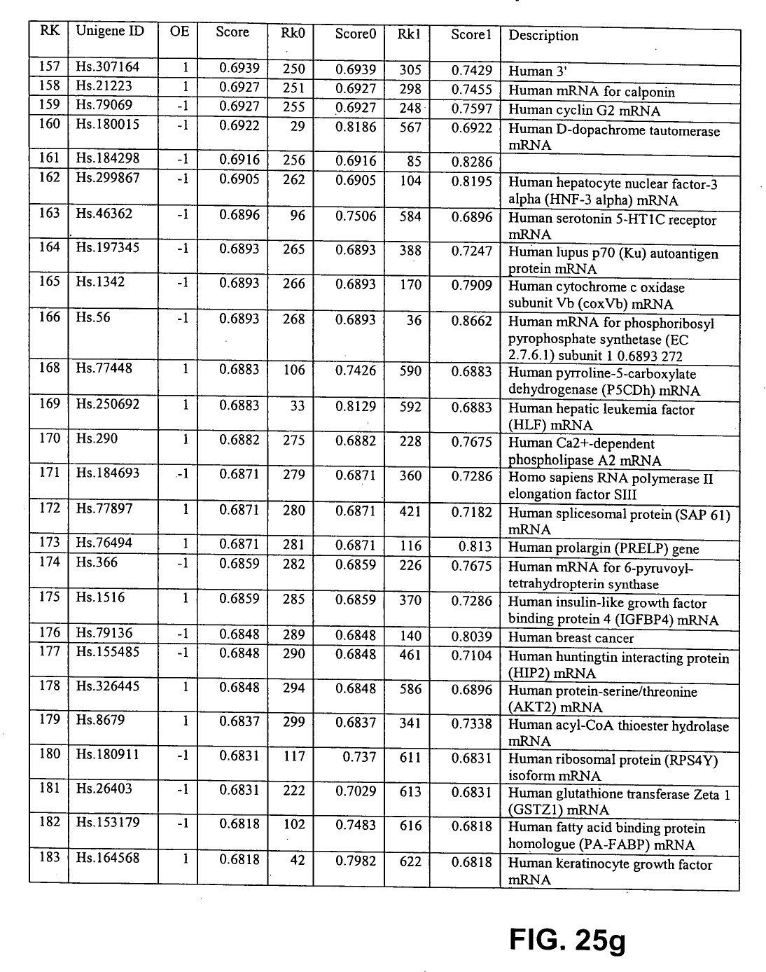

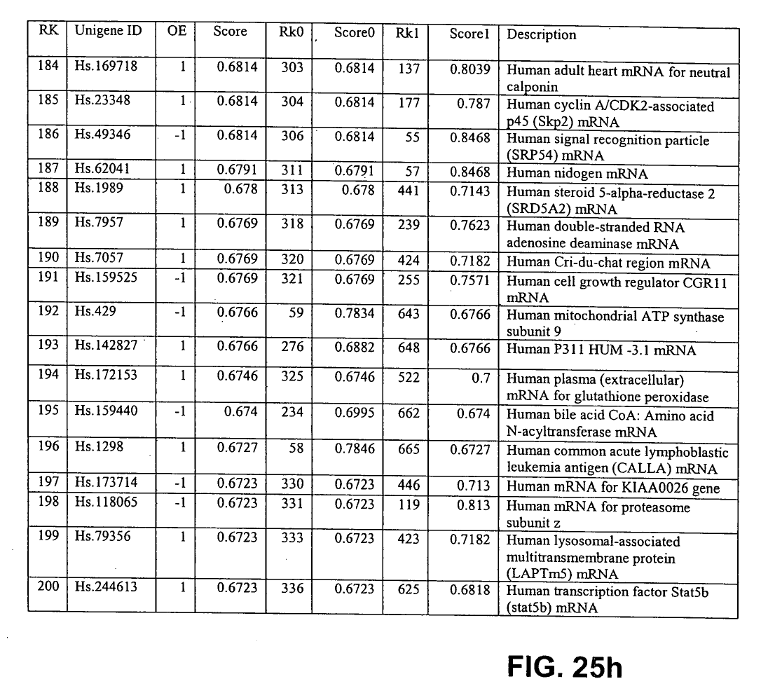

The same procedures as above were repeated for the separation of BPH vs. all other tissues. The correlation between the scores of the genes obtained in both studies was investigated. The Pearson correlation is R=0.37, smaller than the value 0.46 found in the separation tumor vs. others. FIG. 24 provides the tables of genes ranked by either study for BPH vs. others. The genes are ranked in two ways, using the data of the first study (2001) and using the data of the second study (2003). The genes are ranked according to a score that is the minimum of score 0 and score 1. FIG. 25 lists the BPH vs. others feature ranking by consensus between the 2001 study and the 2003 study.

There are only 17 BPH samples in the first study and only 10 in the second study. Hence, the pvalues obtained are not as good. Further, in the 2001 study, very few non-tumor samples are not BPH: 8 NL, 1 stroma, 3 Dysplasia. Therefore, the gene selection from the 2001 study samples is biased toward finding genes that separate well tumor vs. BPH and ignore the other controls.

As before, one dataset was used as training set and the other as test set, then the two datasets were swapped. This time, we get significantly better results by training on the study 1 data and testing on the study 0 data. This can be explained by the fact that the first study included very few control samples other than BPH, which biases the feature selection.

Training on the 2003 study and testing on the 2001 study for 10 features yields about 10% error. This is not as good as the results obtained by cross-validation, where there was only one error, but still quite reasonable. Lesser results using an independent test set were expected since there are only 10 BPH samples in the 2003 study.

When the features are selected with the samples of the 2001 study, the normal samples are grouped with BPH in the 2003 study, even though the goal was to find genes separating BPH from all others. When the features are selected with the 2003 study samples, the BPH samples of study 0 are not well separated.

In conclusion, it was not obvious that there would be agreement between the genes selected using two independent studies that took place at different times using different arrays. Nonetheless, there was a significant overlap in the genes selected. Further, by training with the data from one study and testing on the data from the other good classification performances were obtained both for the tumor vs. others and the BPH vs. others separations (around 10% error). To obtain these results, the gene set was limited to only 2000 genes. There may be better candidates in the genes that were discarded, however, the preference was for increased confidence in the genes that have been validated by several studies.

### EXAMPLE 7

### Validation of the Stamey Study with Publicly Available Data (01/2005)

In this example, five publicly available datasets containing prostate cancer samples processed with an Affymetrix chip (chip U95A) are merged to produce a set of 164 samples (102 tumor and 62 normal), which will be referred to as the “public data” or “public dataset”. The probes in the U95A (˜12,000 probes) chip are matched with those of the U133A chip used in the 87 sample, 2003 Stamey study (28 tumor, 49 normal, ˜22000 probes) to obtain approximately 7,000 common probes.

The following analysis was performed for the Tumor vs. Normal separation:

Selection of genes uses the AUC score for both the public data set and the Stamey dataset. The literature analysis of the top consensus genes reveals that they are all relevant to cancer, most of them directly to prostate cancer. Commercial antibodies to some of the selected proteins exist.

Training is done on one dataset and testing on the other with the Golub classifier. The balanced classification success rate is above 80%. This increases to 90% by adapting only 20 samples from the same dataset as the test set.

Several datasets were downloaded from the Internet (Table 32 and Table 33). The Oncomine website, on the Worldwide Web at oncomine.org, is a valuable resource to identify datasets, but the original data was downloaded from the author's websites. Table 32 lists Prostate cancer datasets and Table 33 is Multi-study or normal samples.

TABLE 32

| Name | Chip | Samples | Genes | Ref. | Comment |
|---|---|---|---|---|---|
| Febbo | U95A v2 | 52 tumor 50 normal | ˜12600 | [1] | Have data. |
| Dhana | cDNA | Misc ˜40 | 10000 | [2] | Difficult to understand |
| | | | | | and read data. |
| LaTulippe | U95A | 3 NL, 23 localized and | ˜12600 | [3] | Have data. |
| | | 9 metastatic | | | |
| LuoJH | Hu35k | 15 tumor, 15 normal | ˜9000 | [4] | Have data. Some work |
| | | | | | to understand it. |
| McGee | Hu6800 | 8 primary, 3 metastasic | 6800 | [5] | Not worth it. |
| | Ge | and 4 nonmalignant | | | |
| Welsh | U95A | 9 normal, 24 localized and 1 | ˜12000 | [6] | Looks OK. |
| | | metastatic, and 21 cell lines | | | |
| LuoJ | cDNA | 16 tumor 9 BPH | ˜6500 | [7] | Probably not worth it. |

TABLE 33

| Name | Chip | Samples | Genes | Ref. | Comment |
|---|---|---|---|---|---|
| Rama | Hu6800 | 343 primary and | ˜16000 | [8] | Looks |
| | Hu35kSubA | 12 metastatic; | | | interesting. |
| | | include a few | | | Complex data. |
| | | prostate | | | |
| Hsiao | HuGenFL | 59 normal | ˜10000 | [9] | Looks good. |
| | | | | | Same chips as |
| | | | | | Stamey 2001. |
| Su | U95a | 175 tumors, | ˜12600 | [10] | Looks good. |
| | | of which 24 | | | |
| | | prostate | | | |

The datasets of Febbo, LaTulippe, Welsh, and Su are formatted as described below because they correspond to a large gene set from the same Affymetrix chip U95A.

Febbo Dataset

File used:
Prostate_TN_final0701_allmeanScale.res
A data matrix of 102 lines (52 tumors, 50 normal) and 12600 columns was generated.
All samples are tumor or normal. No clinical data is available.
   LaTulippe Dataset—
The data was merged from individual text files (e.g. MET1_U95Av2.txt), yielding to a data matrix of 35 lines (3 normal, 23 localized, 9 metastatic) and 12626 columns. Good clinical data is available.
   Welsh Dataset

The data was read from file:
GNF_prostate_data_CR61_{—}5974.xls
A matrix of 55 lines (9 normal, 27 tumor, 19 cell lines) and 12626 lines was generated. Limited clinical data is available. Some inconsistencies in tissue labeling between files.
   Su Dataset

The data was read from: classification_data.txt
A matrix of 174 lines (174 tumors of which 24 prostate) and 12533 lines was obtained. No clinical data available.

The initial analysis revealed that the Su and Welsh data were identical, so the Su dataset was removed.

TABLE 34

| | | | | | Stamey |
|---|---|---|---|---|---|
| | Febbo | LaTulippe | Welsh | Su | 2003 |
| Febbo | 12600 | 12600 | 12600 | 12533 | 312 |
| LaTulippe | 12600 | 12626 | 12626 | 12533 | 312 |
| Welsh | 12600 | 12626 | 12626 | 12533 | 312 |
| Su | 12533 | 12533 | 12533 | 12533 | 271 |
| Stamey | 312 | 312 | 312 | 271 | 22283 |

From Table 34, it can be verified that the four datasets selected use the same chip (Affymetrix U95A). The Stamey data however uses a different chip (Affymetrix U133A). There are only a few probes in common. Affymetrix provides a table of correspondence between probes corresponding to a match of there sequence.

Using Unigene IDs to find corresponding probes on the different chips identified 7350 probes. Using the best match from Affymetrix, 9512 probes were put in correspondence. Some of those do not have Unigene IDs or have mismatching Unigene IDs. Of the matched probes, 6839 have the same Unigene IDs; these are the ones that were used.

The final characteristics of publicly available data are summarized in Table 35. The other dataset used in this study is the prostate cancer data of Stamey 2003 (Table 36). The number of common gene expression coefficients used is n=6839. Each dataset from the public data is preprocessed individually using the script my_normalize, see below.

For preprocessing, a bias of zero was used for all normalizations, which were run using the following script:

function X=my_normalize(X, bias)

if nargin<2, bias=0; end

mini=min(min(X));

maxi=max(max(X));

X=(X-mini)/(maxi-mini)+bias;

idx=find(X<=0);

X(idx)=Inf;

epsi=min(min(X));

X(idx)=epsi;

X=log(X);

X=med_normalize(X);

X=med_normalize(X′)′;

X=med_normalize(X);

X=med_normalize(X′)′;

X=tanh(0.1*X);

function X=med_normalize(X)

mu=mean(X,2);

One=ones(size(X,2), 1);

XM=X-mu(:,One);

S=median(abs(XM),2);

X=XM./S(:,One);

The public data was then merged and the feature set is reduced to n. The Stamey data is normalized with my_normalize script after this reduction of feature set. The public data is re-normalized with my_normalize script after this reduction of feature set.

Table 35 shows publicly available prostate cancer data, using U95A Affymetrix chip, sometimes referred to as “study 0” in this example. The Su data (24 prostate tumors) is included in the Welsh data.

TABLE 35

| Data source | Histological classification | Number of samples |
|---|---|---|
| Febbo | Normal | 50 |
| | Tumor | 52 |
| LaTulippe | Normal | 3 |
| | Tumor | 23 |
| Welsh | Normal | 9 |
| | Tumor | 27 |
| Total | | 164 |

Table 36 shows Stamey 2003 prostate cancer study, using U133A Affymetrix chip (sometimes referred to as “study 1” in this example).

TABLE 36

| Prostate zone | Histological classification | Number of samples |
|---|---|---|
| Central (CZ) | Normal (NL) | 9 |
| | Dysplasia (Dys) | 4 |
| | Grade 4 cancer (G4) | 1 |
| Peripheral (PZ) | Normal (NL) | 13 |
| | Dysplasia (Dys) | 13 |
| | Grade 3 cancer (G3) | 11 |
| | Grade 4 cancer (G4) | 18 |
| Transition (TZ) | Benign Prostate Hyperplasia (BPH) | 10 |
| | Grade 4 cancer (G4) | 8 |
| Total | | 87 |

Because the public data does not provide histological details and zonal details, the tests are concentrated on the separation of Tumor vs. Normal. In the Stamey 2003 data, G3 and G4 samples are considered tumor and all the others normal.

The 6839 common genes for the public and the Stamey datsets were ranked independently. The area under the ROC curve was used as ranking criterion. P values (with Bonferroni correction) and False Discovery Rate (FDR) are computed as explained in Example 5 (11/2004).

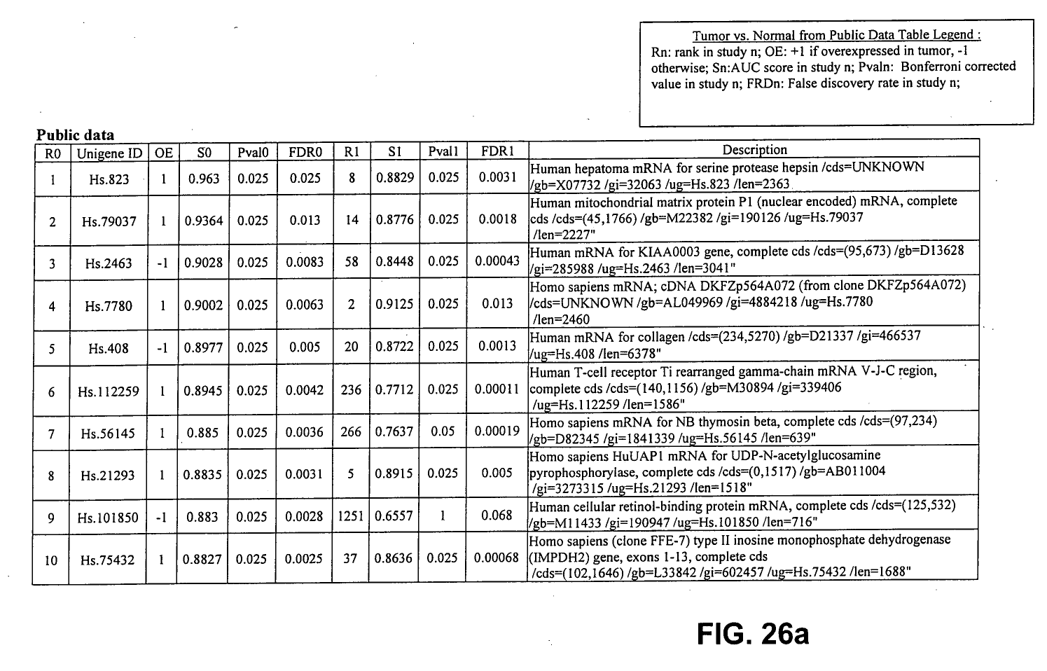

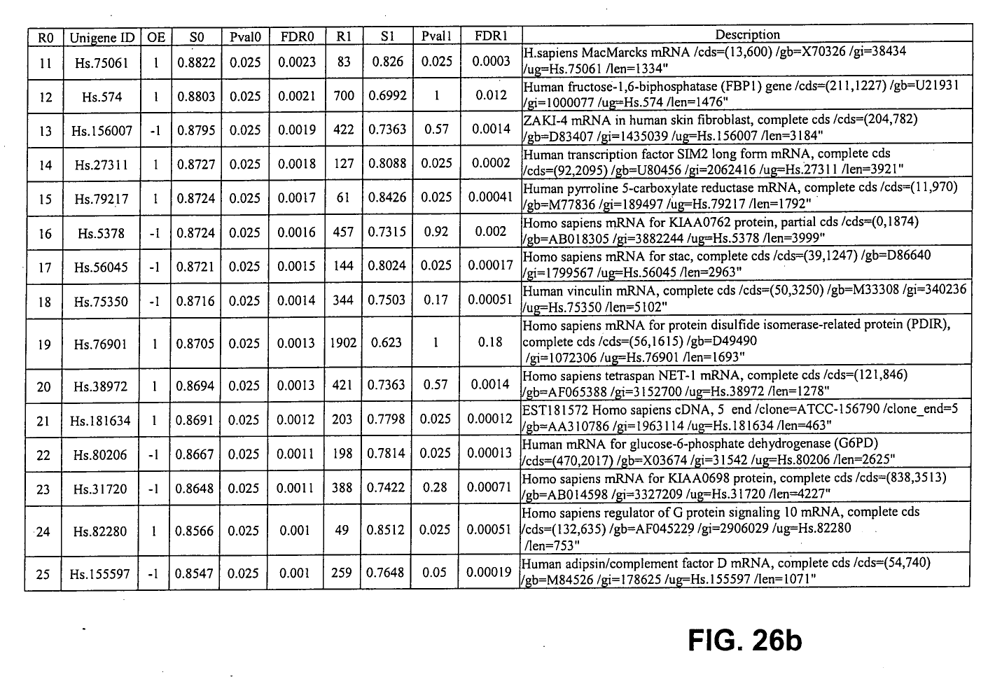

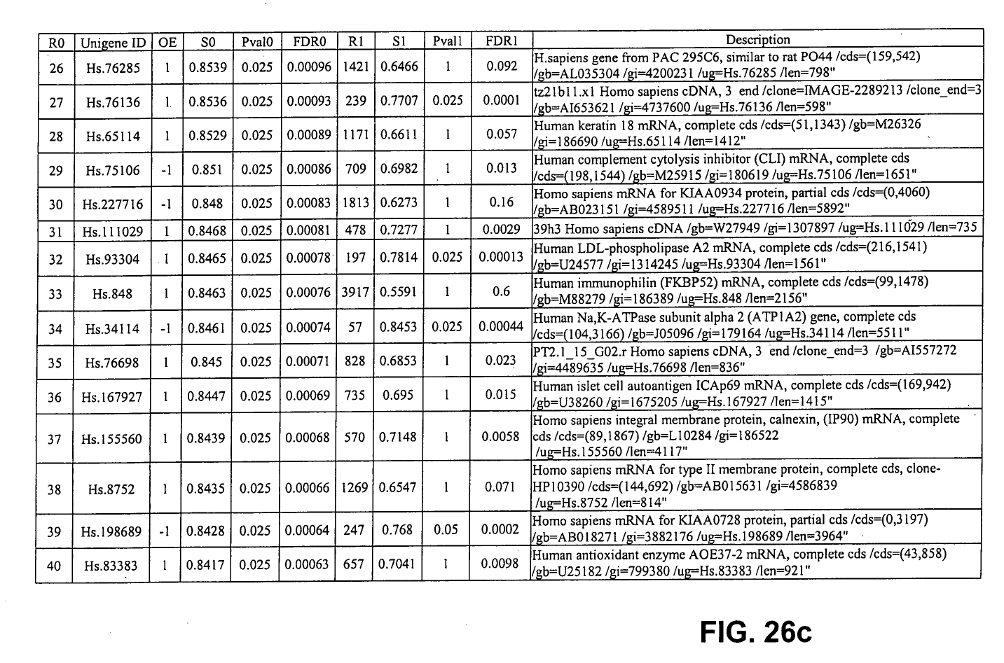

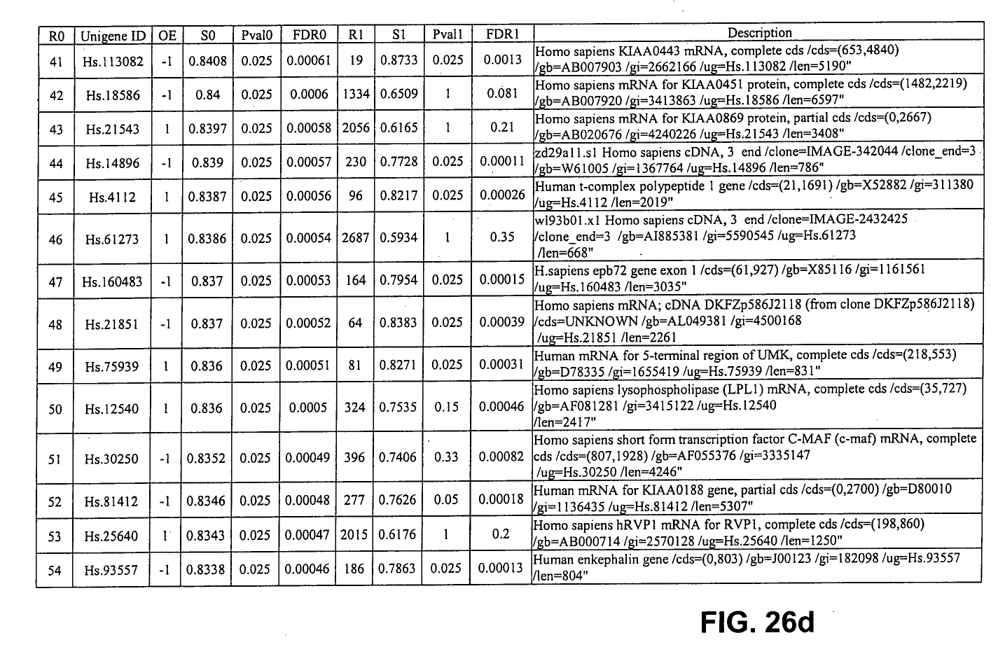

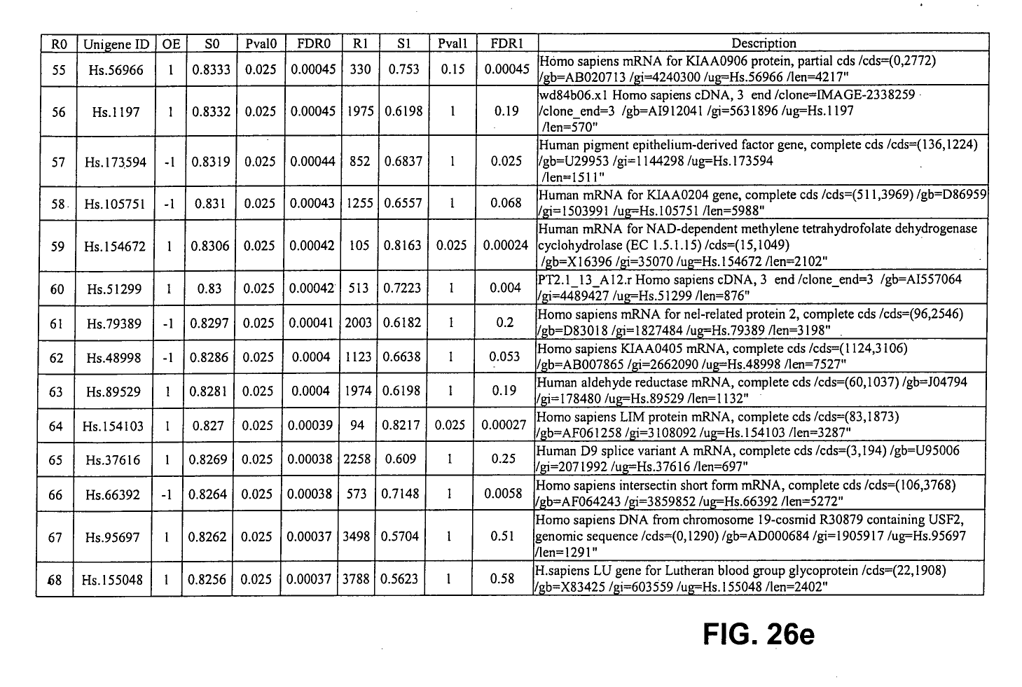

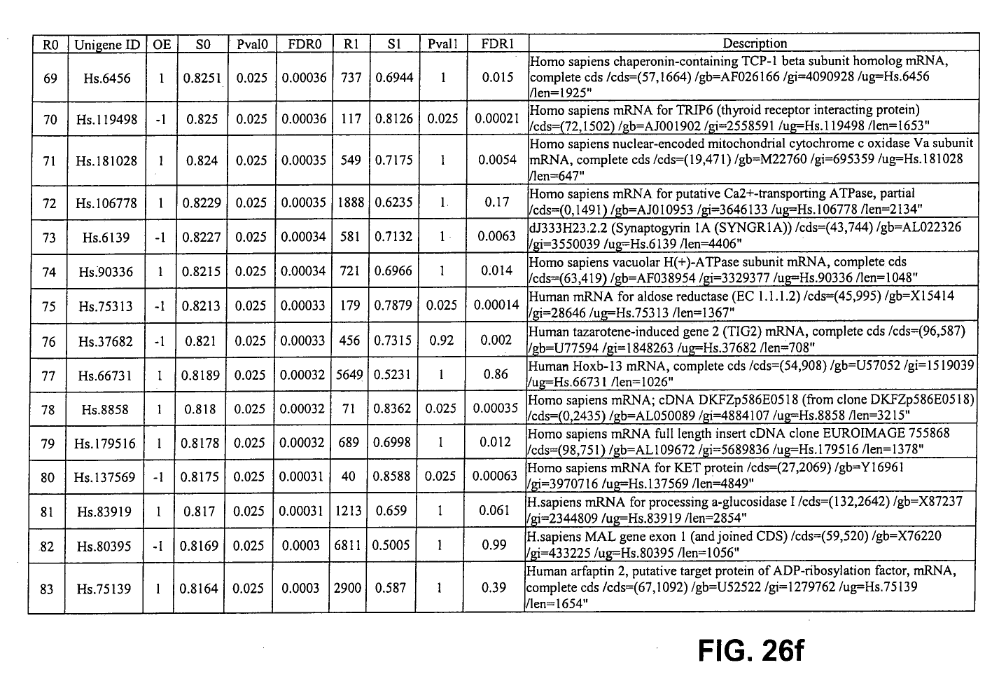

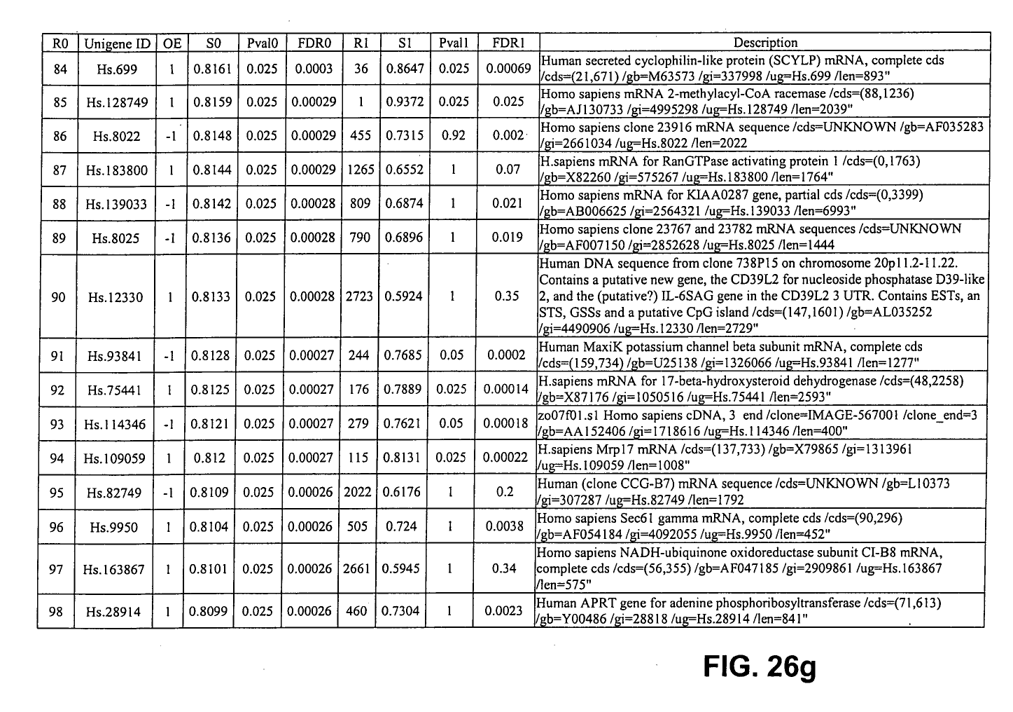

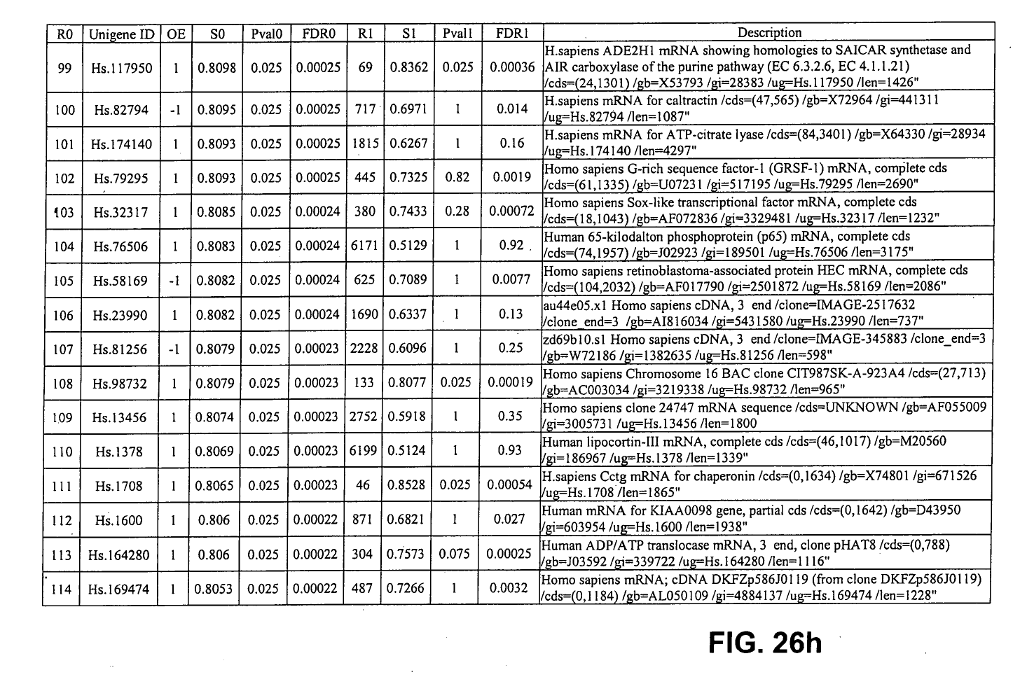

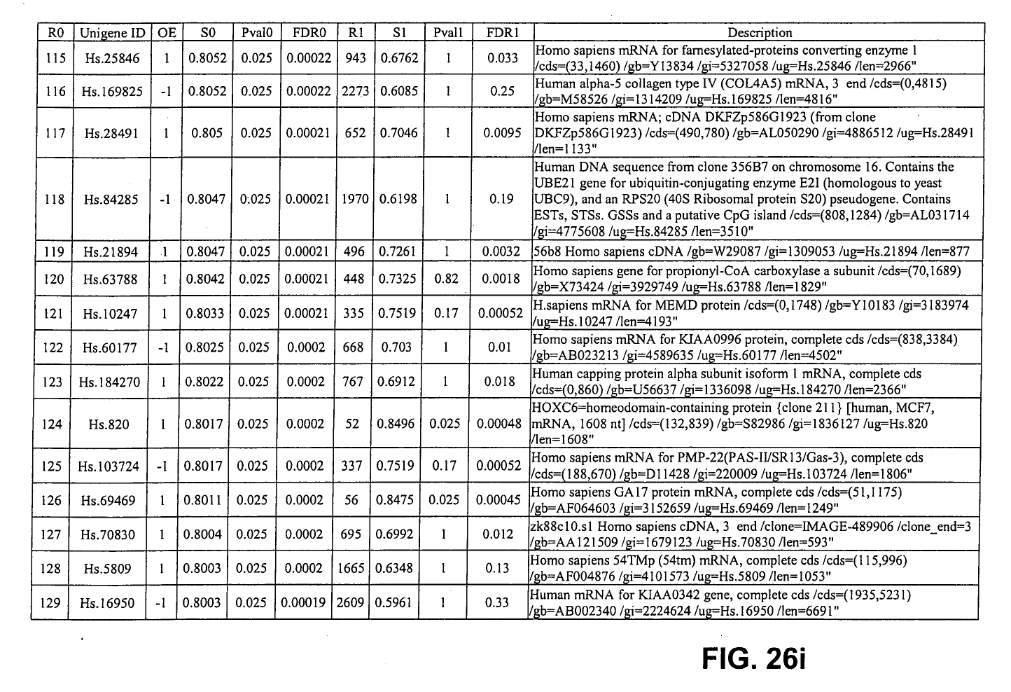

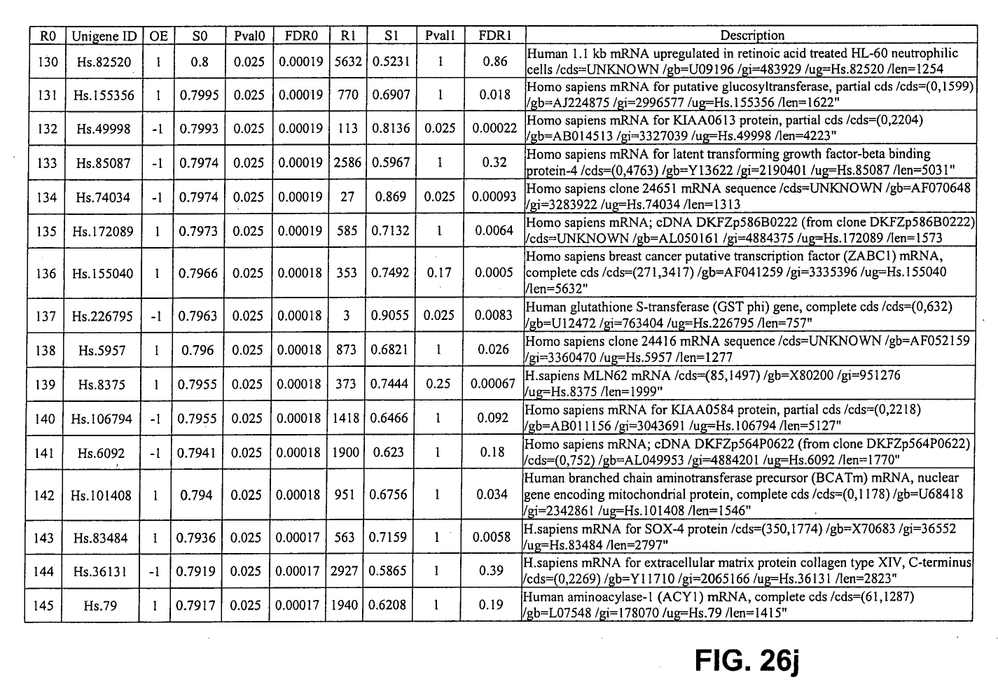

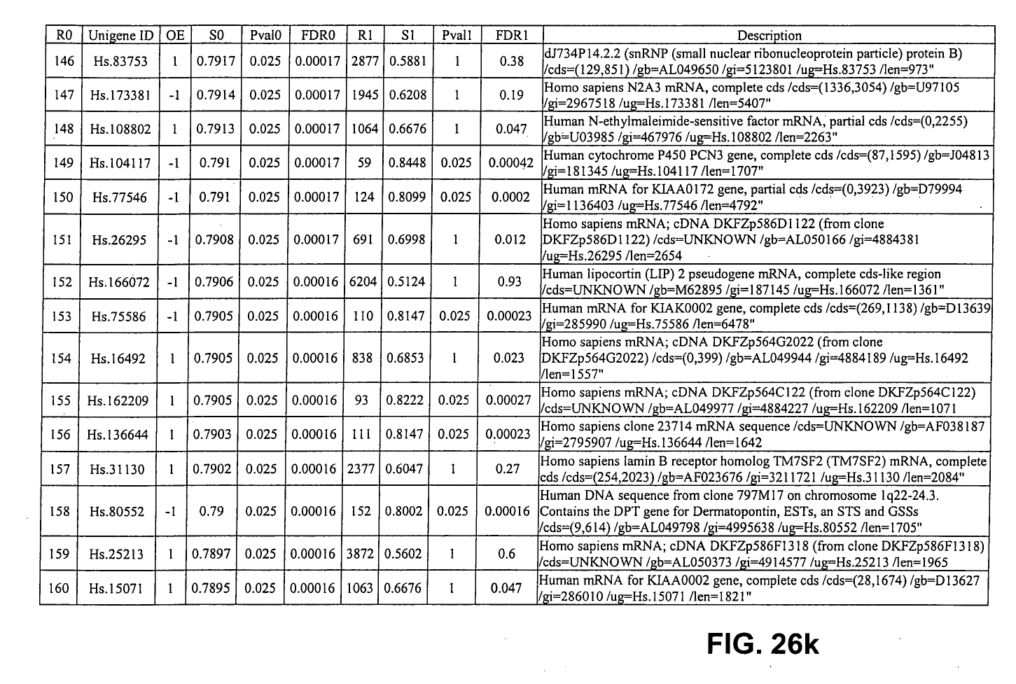

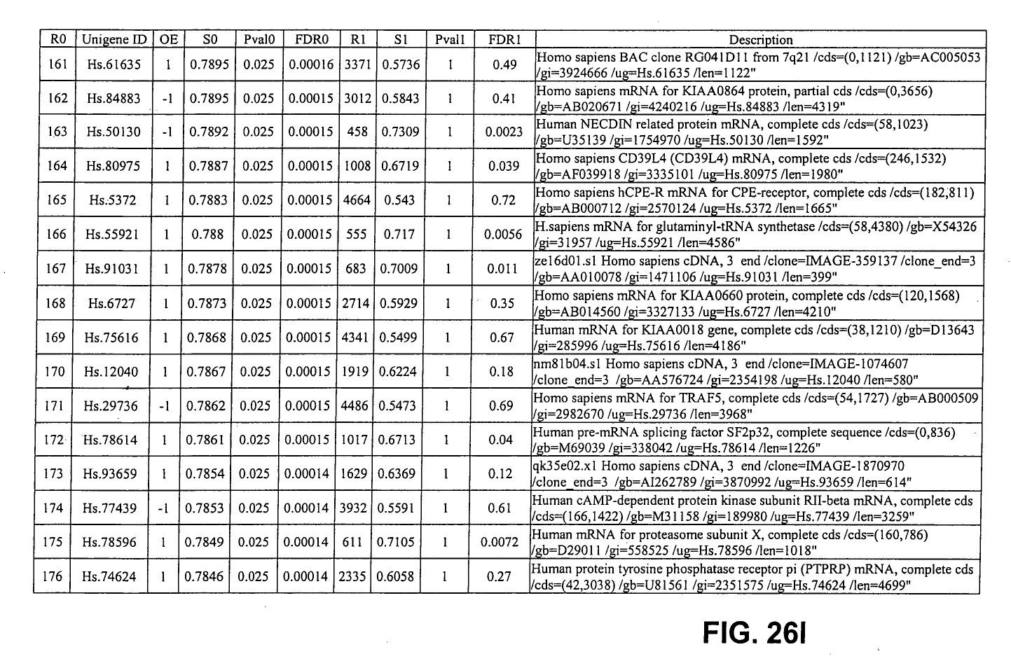

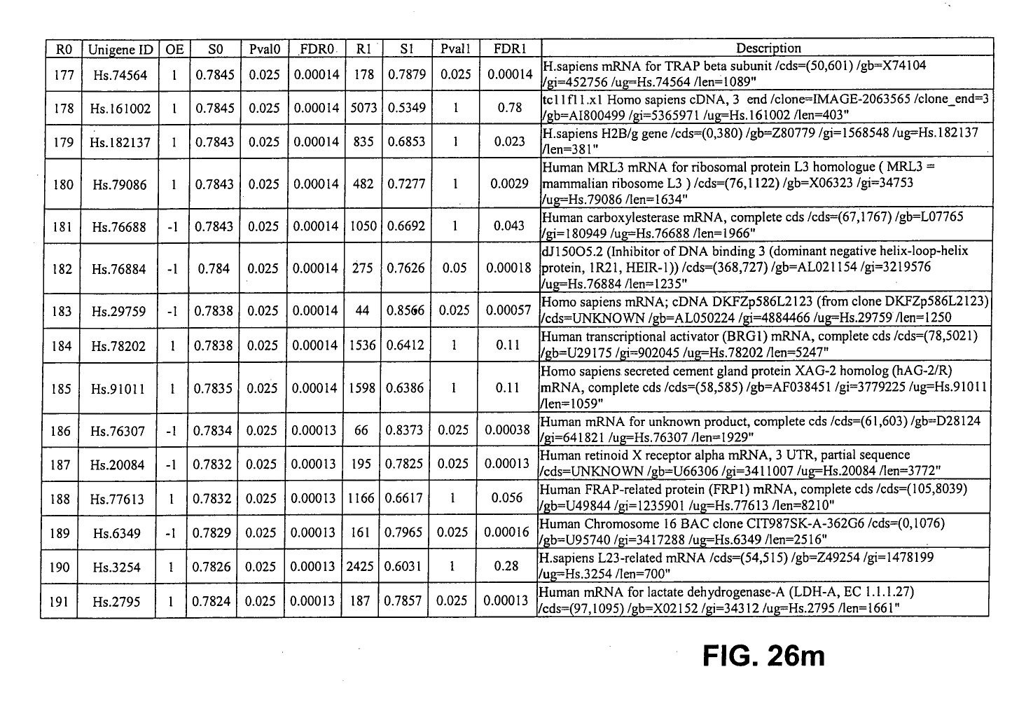

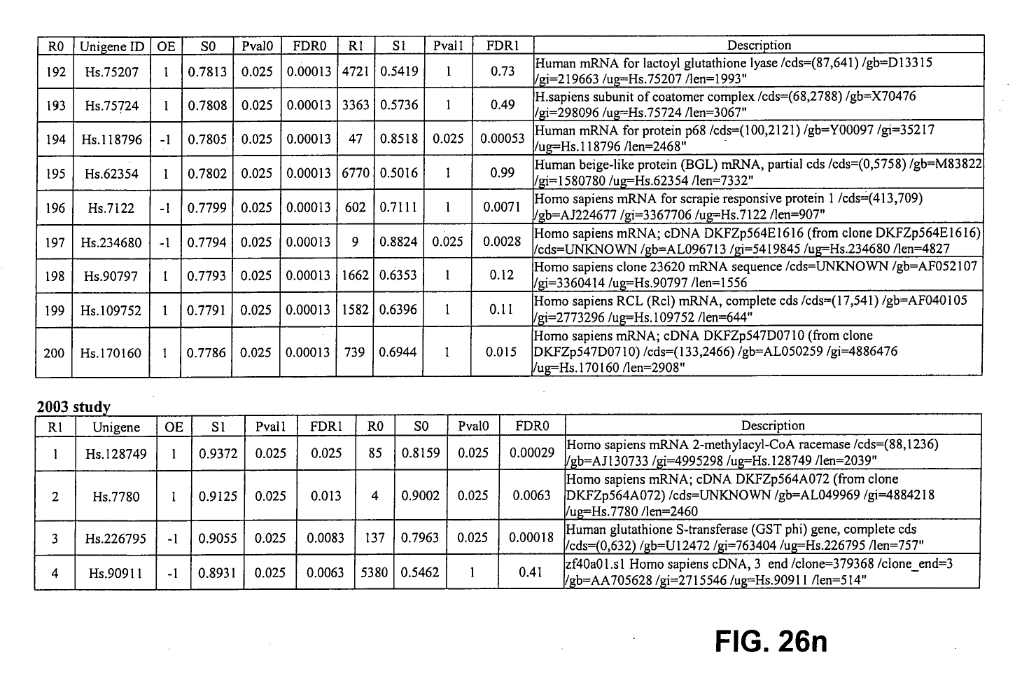

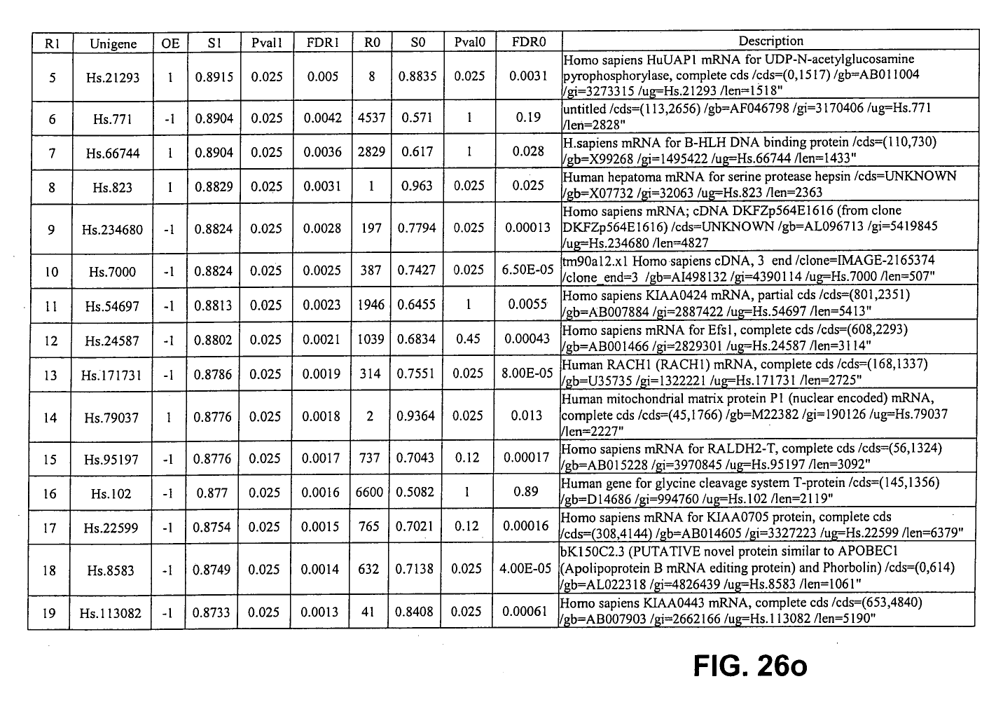

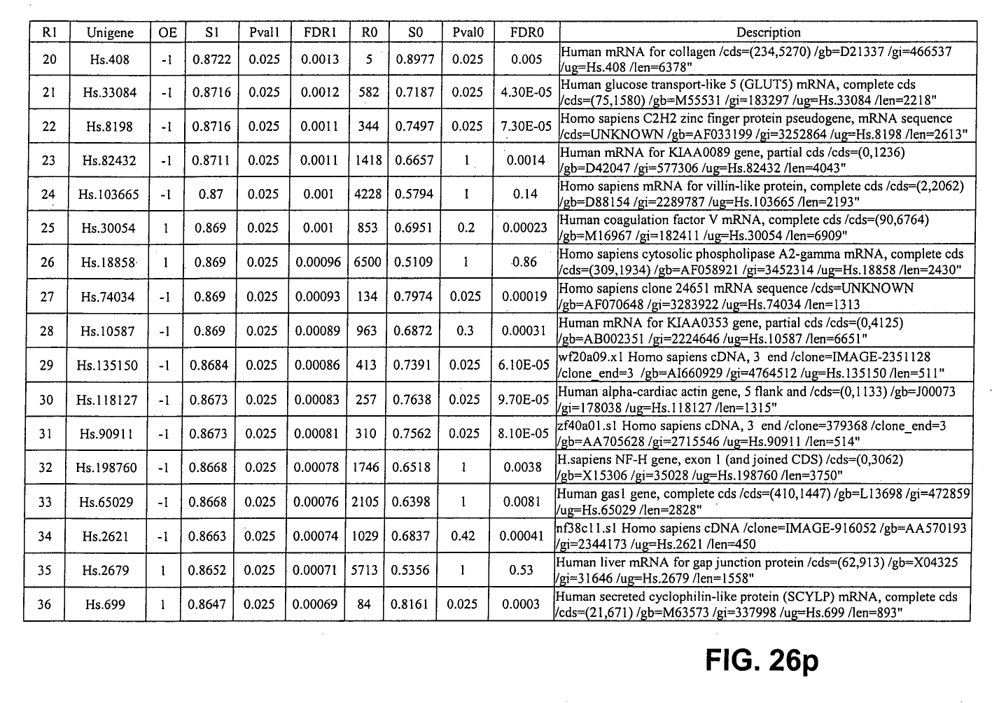

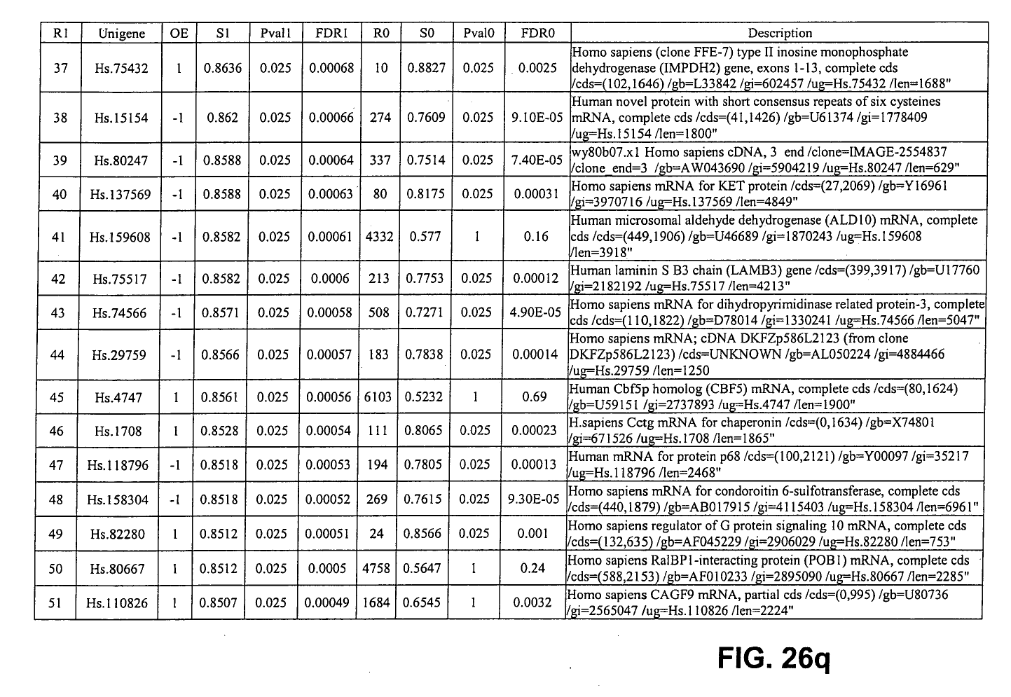

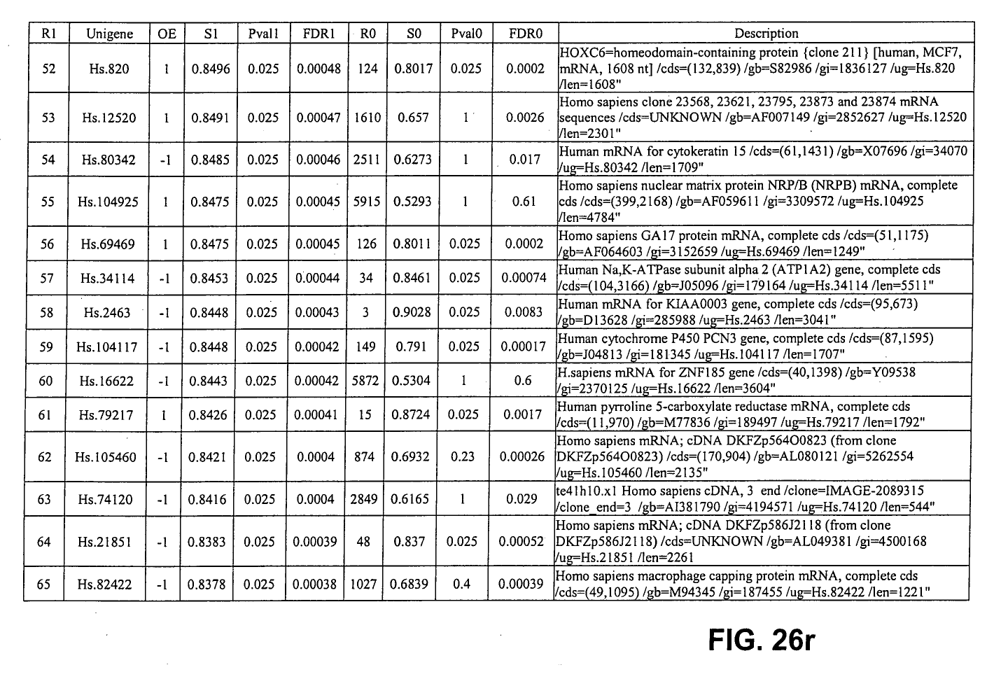

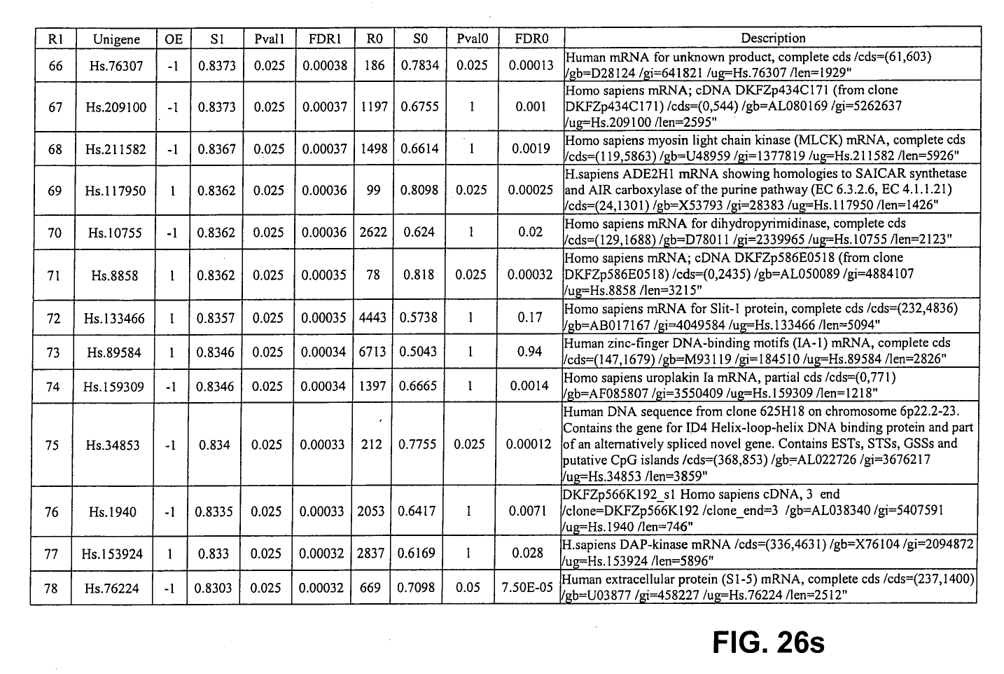

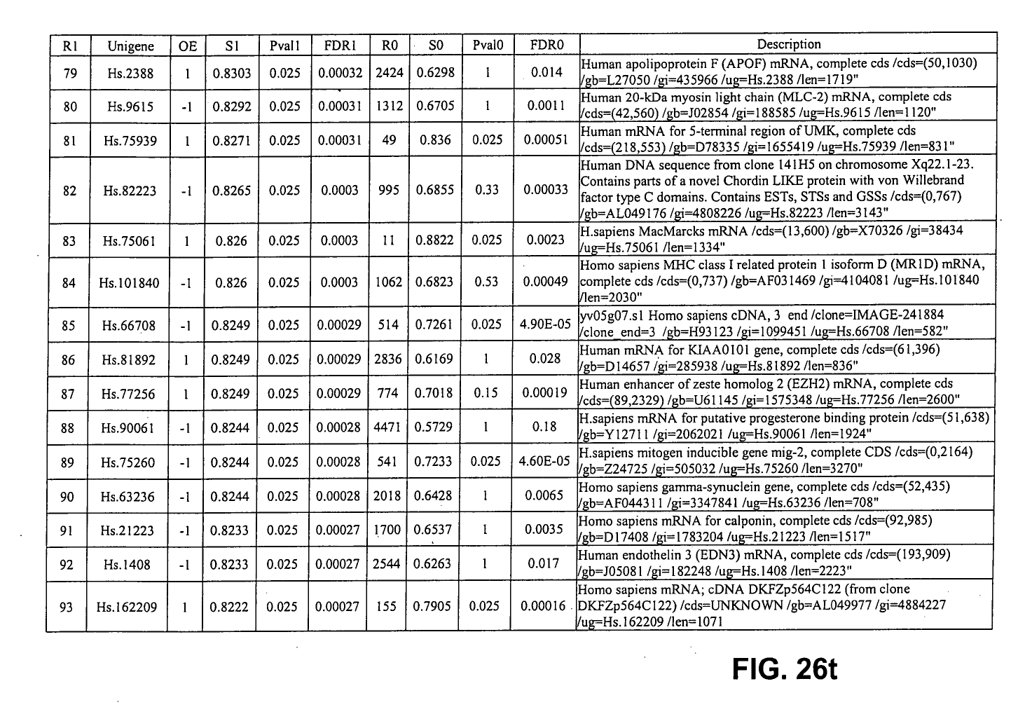

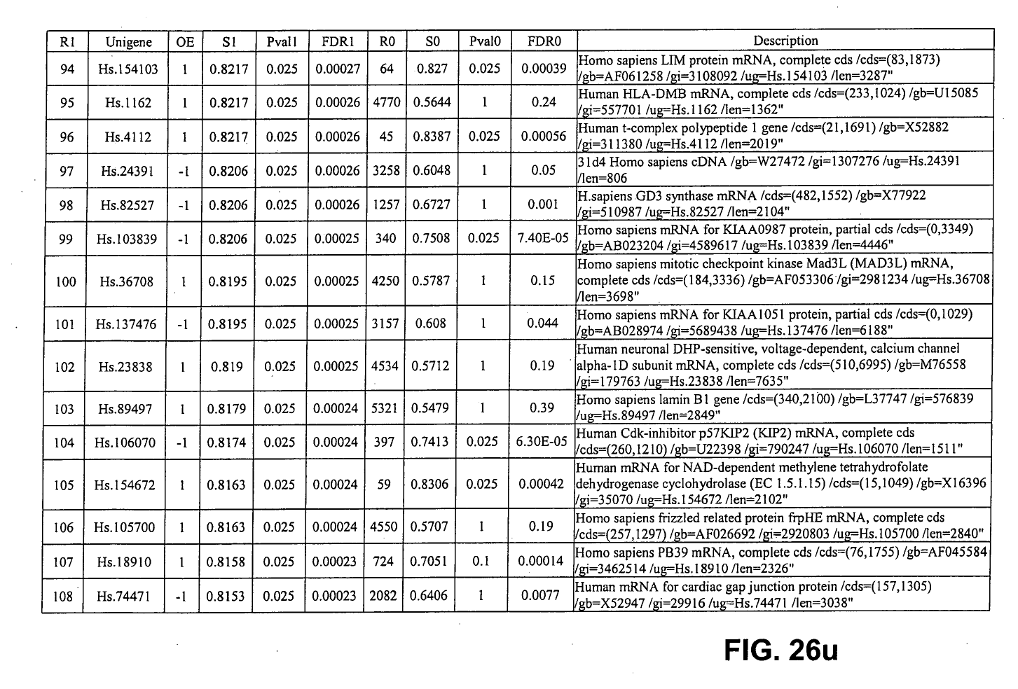

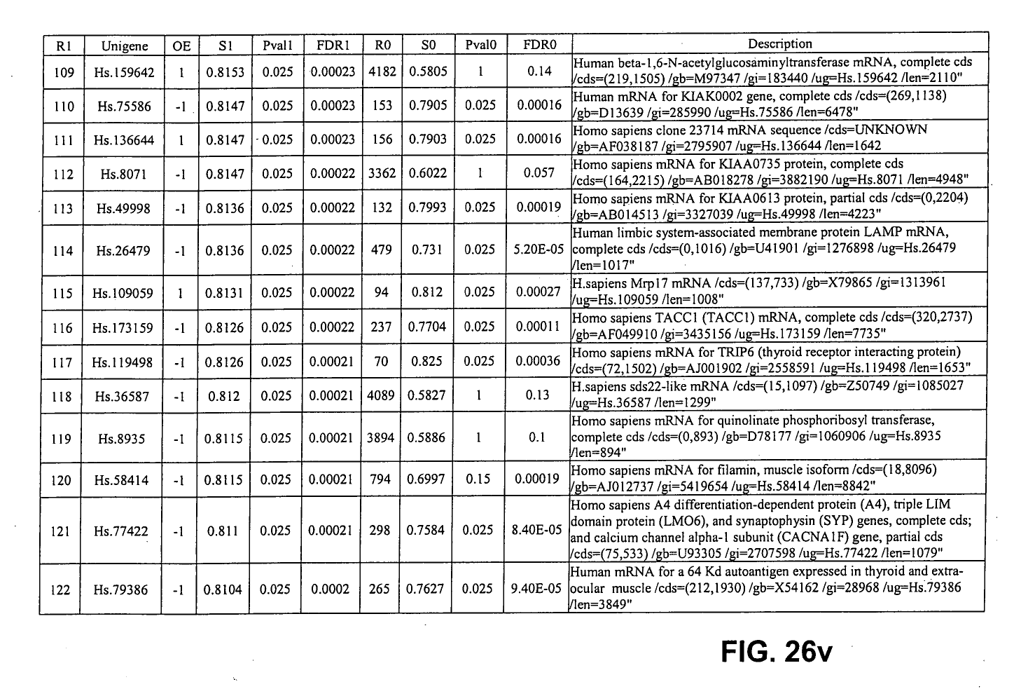

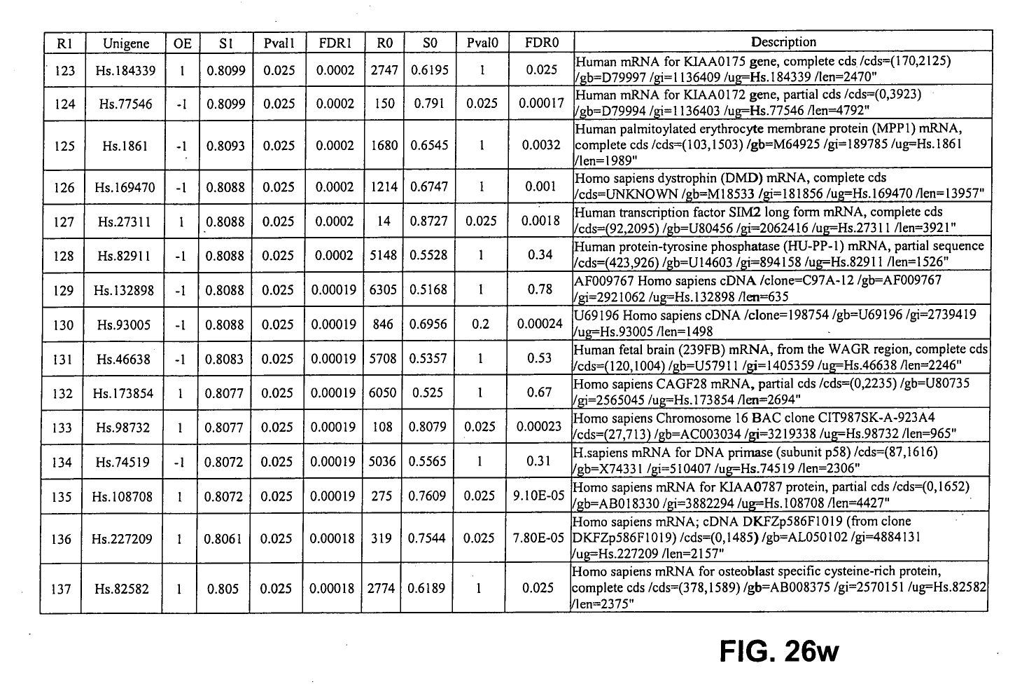

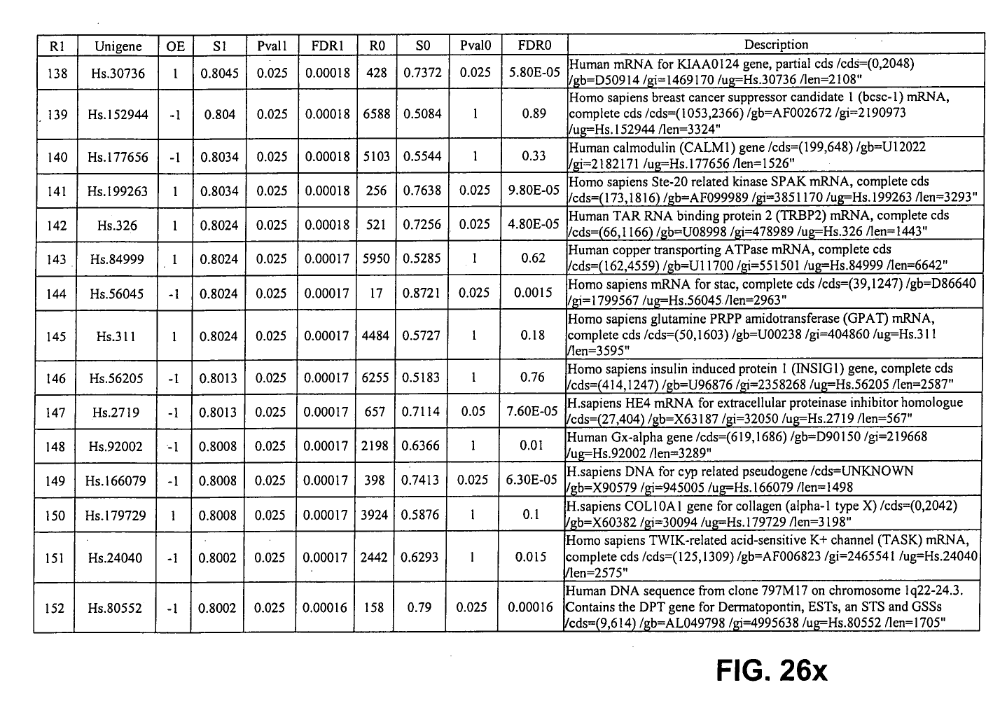

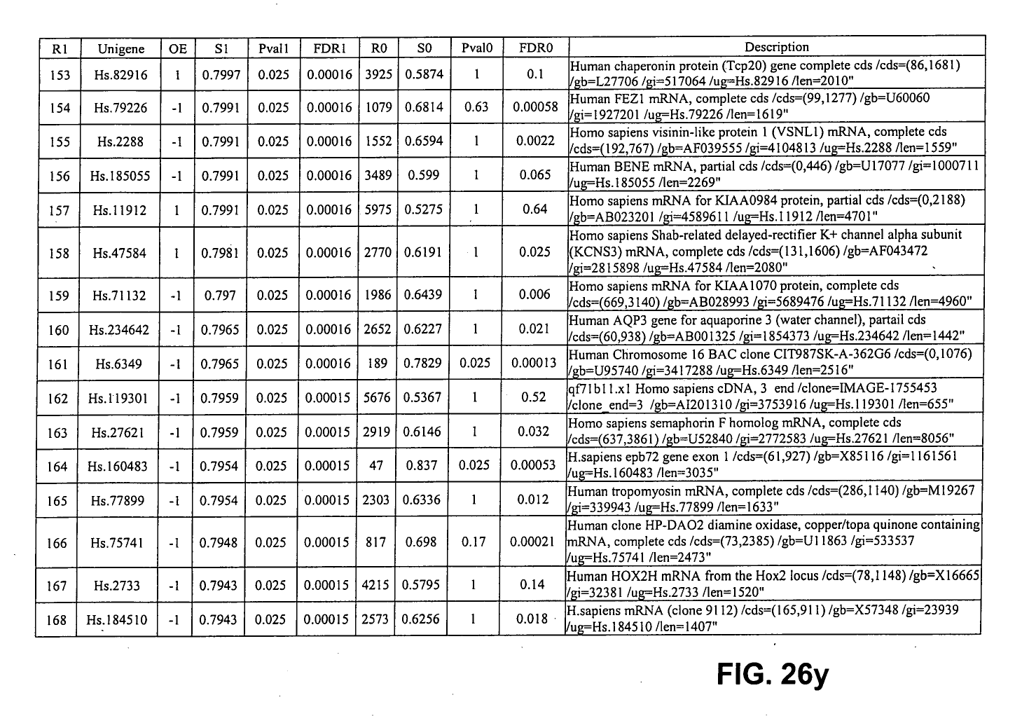

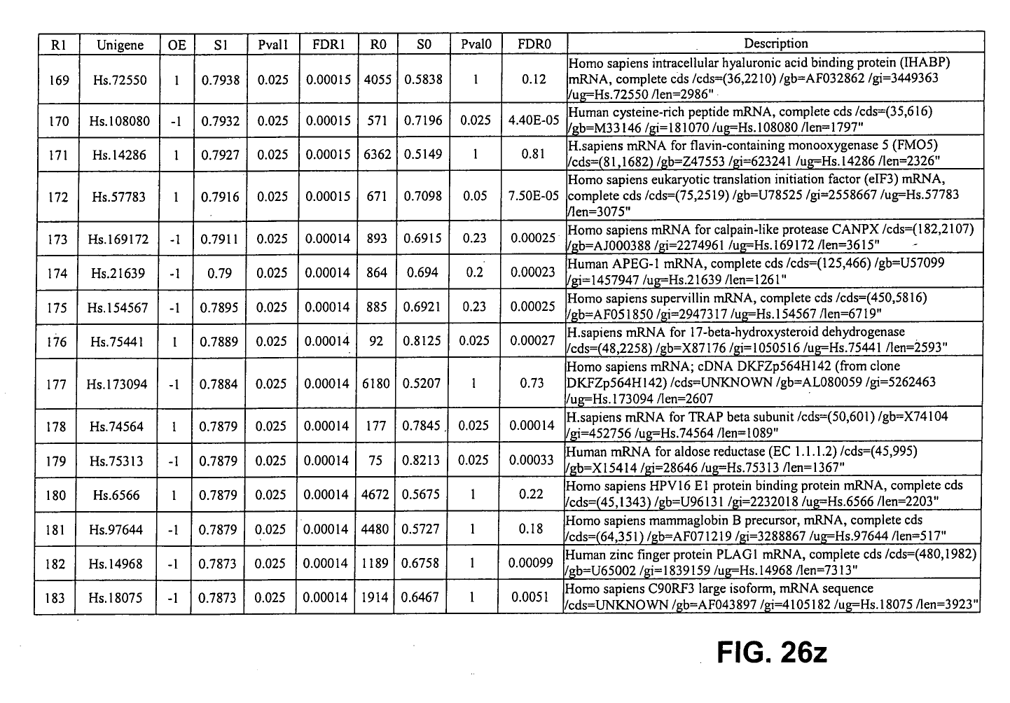

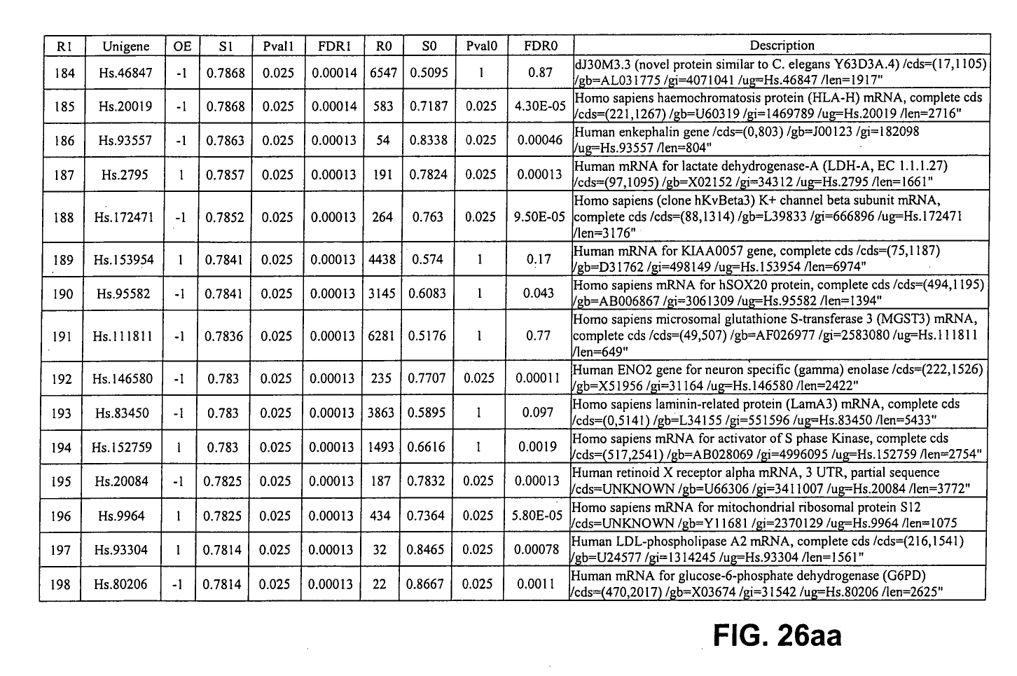

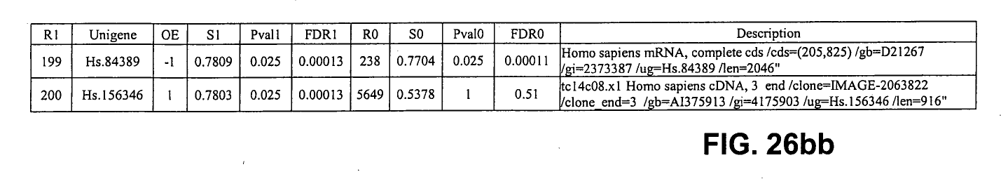

The top 200 genes in each study is presented in the tables in FIG. 26. The genes were ranked in 2 ways, using the data of the first study (study 0=Public data) and using the data of the second study (study 1=Stamey 2003).

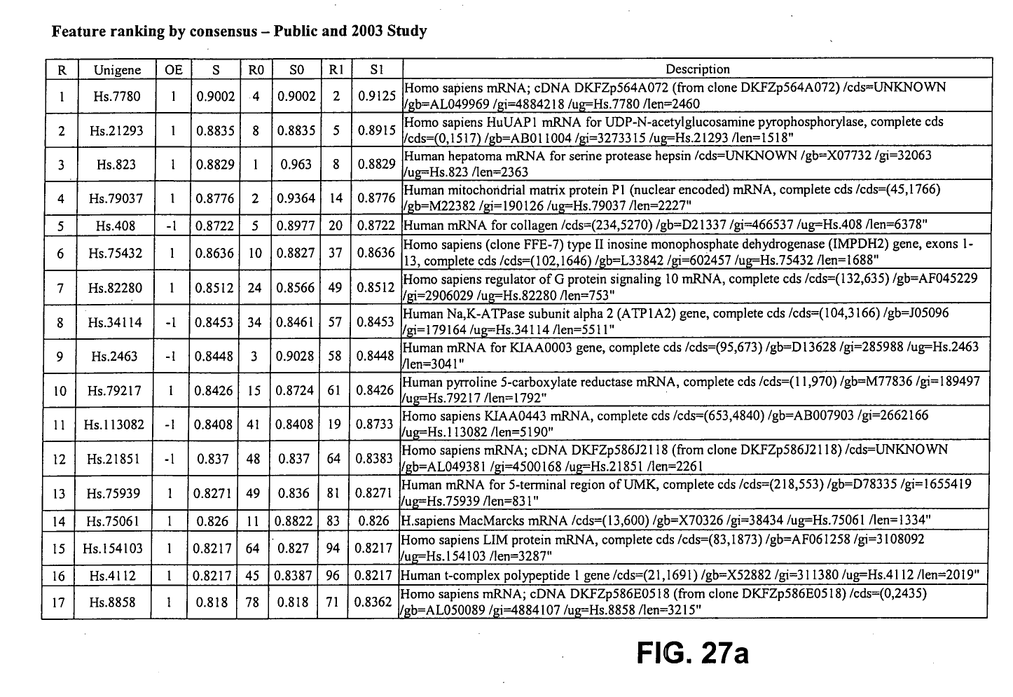

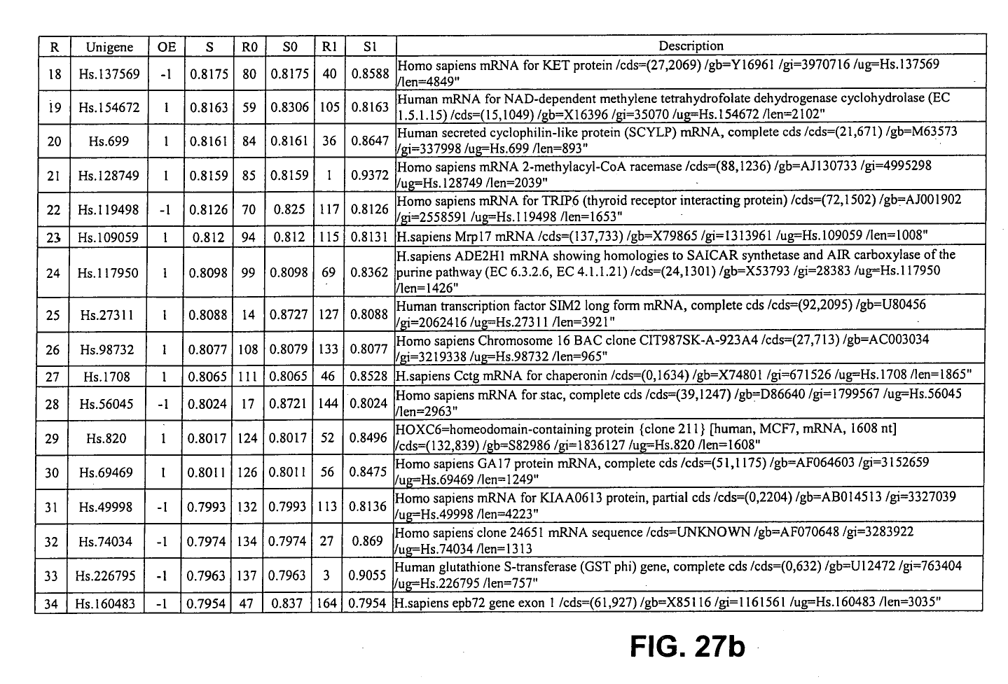

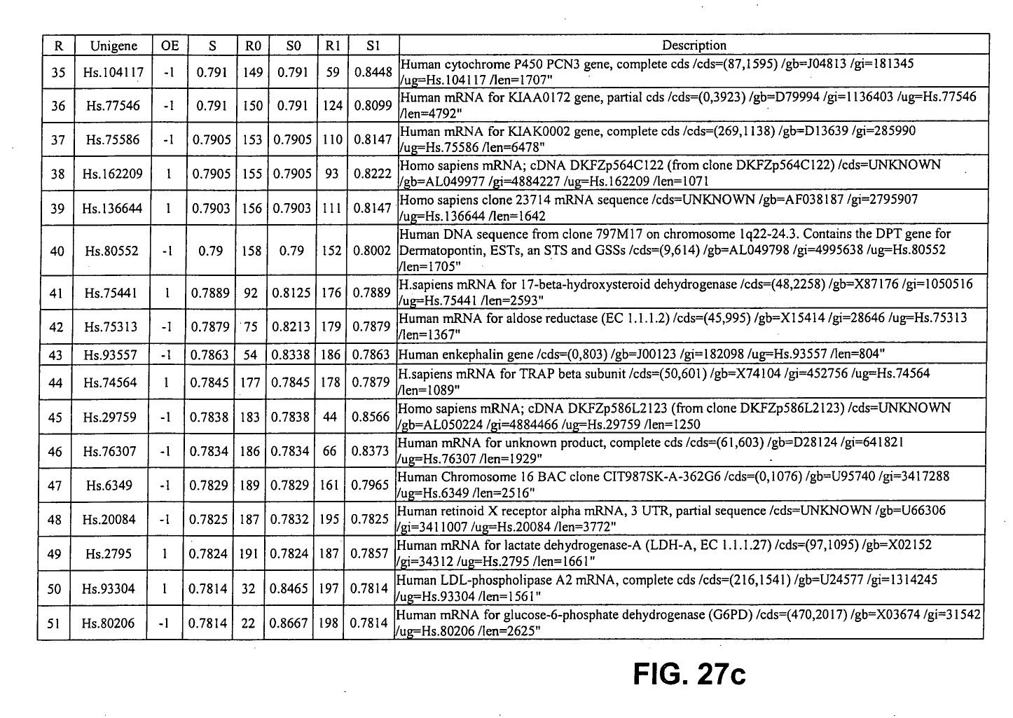

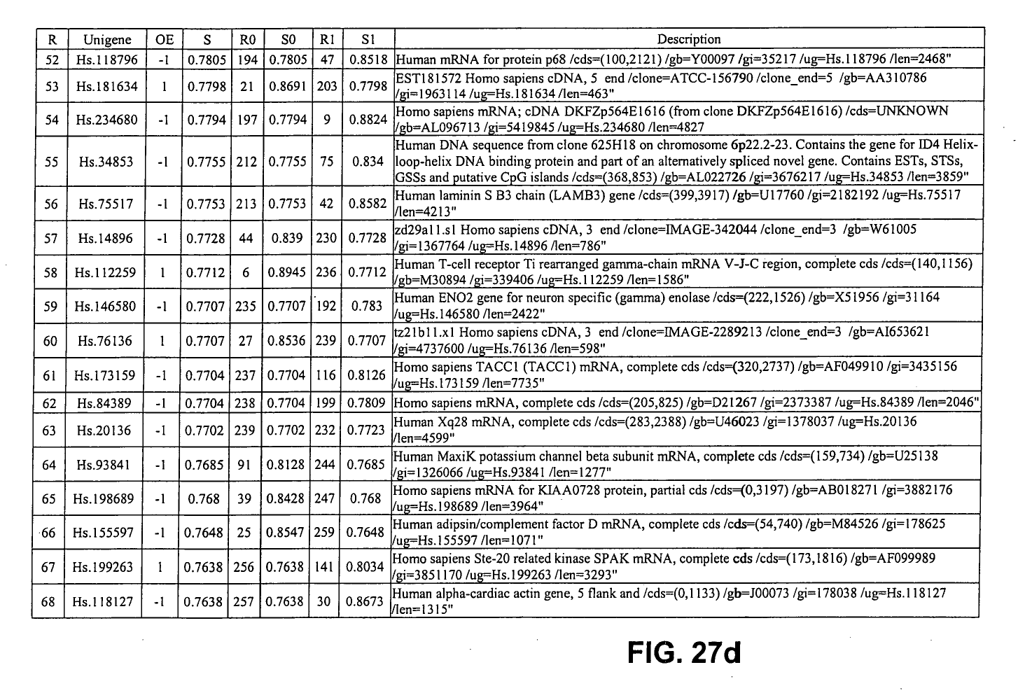

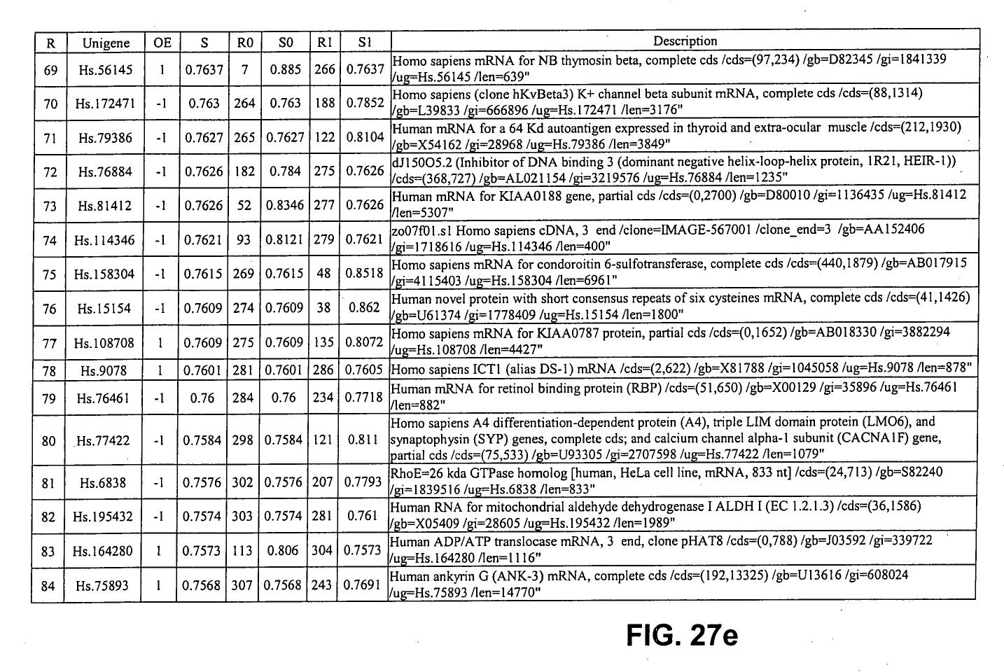

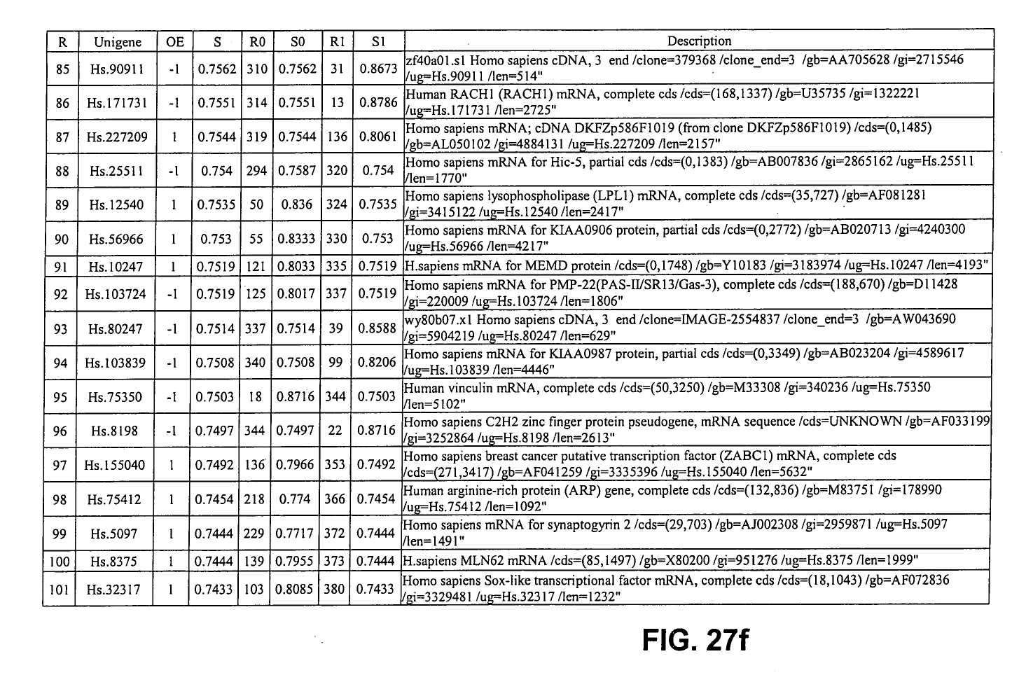

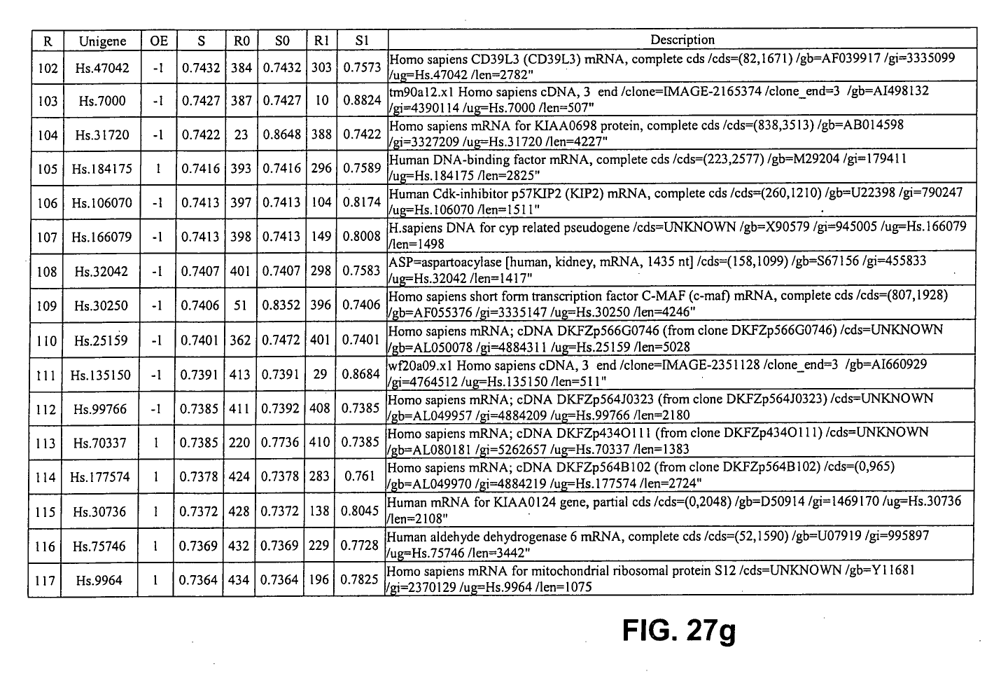

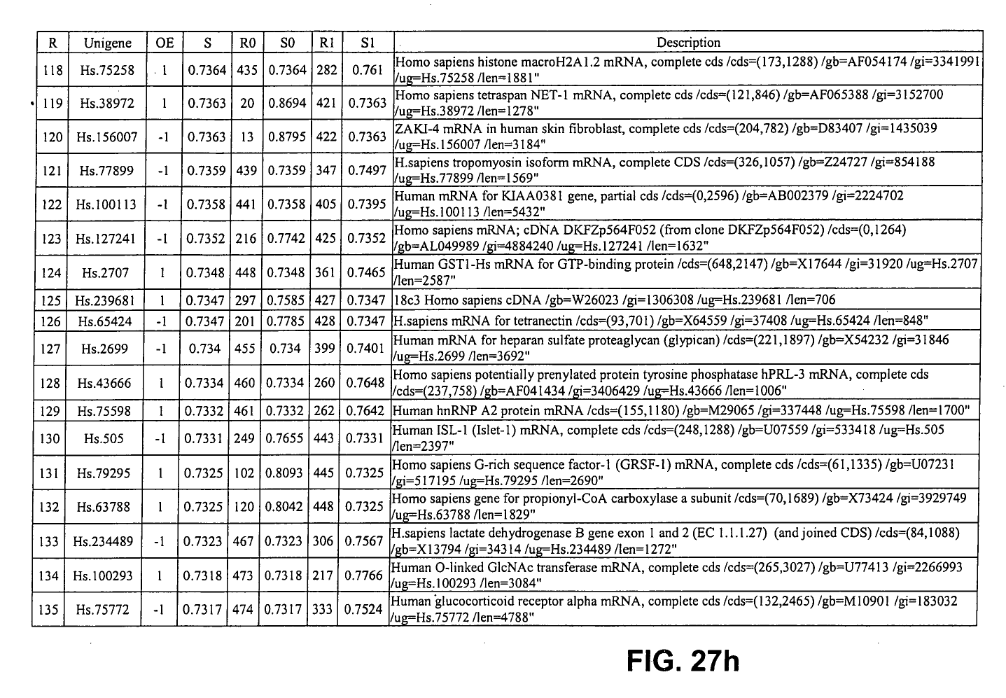

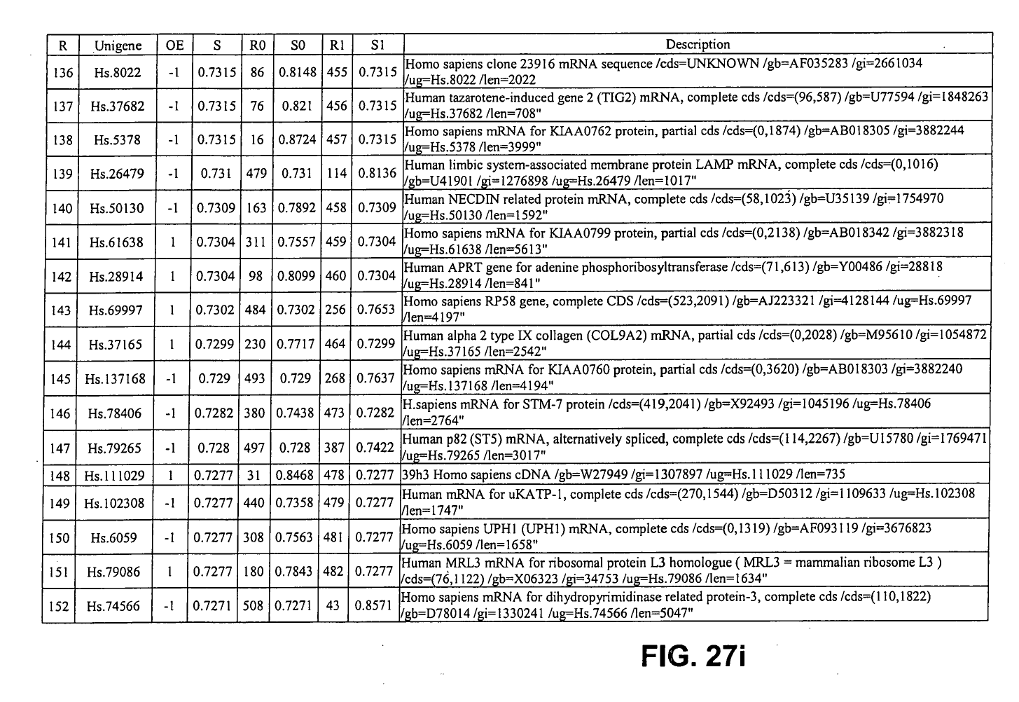

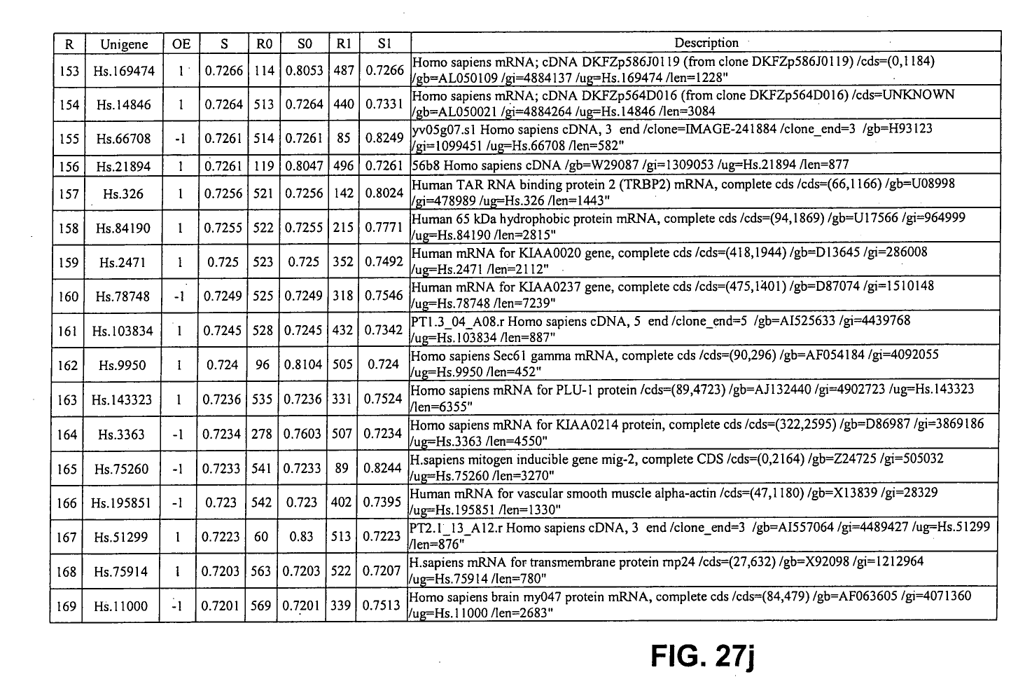

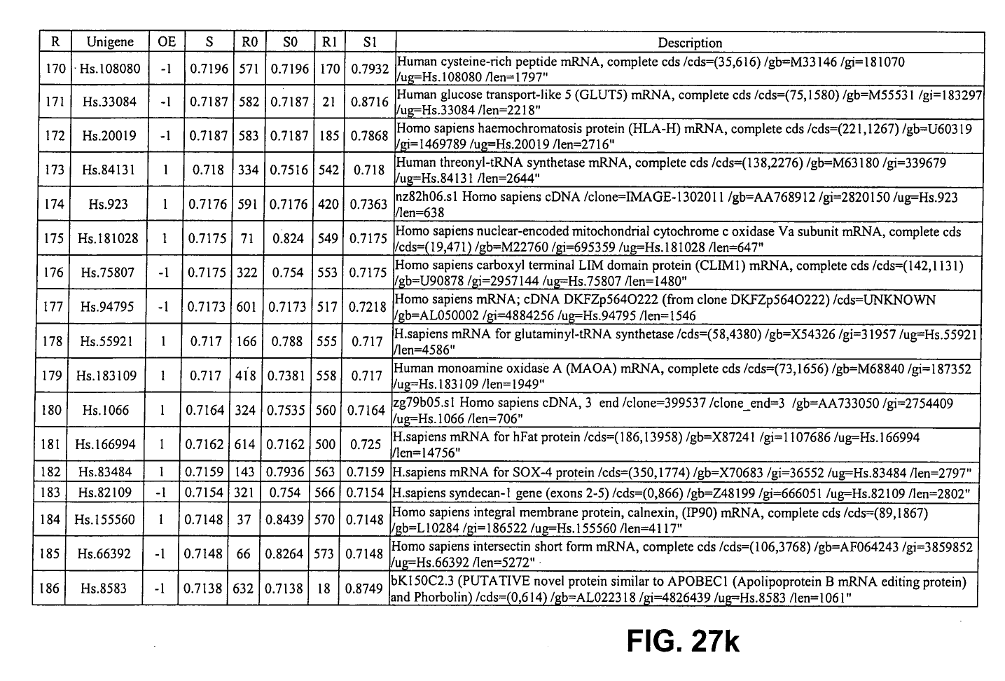

If the public data is ranked, the top ranking genes are more often top ranking in the Stamey data than if the two datasets are reversed. In the table in FIG. 27, genes are ranked according to their smallest score in the two datasets to obtain a consensus ranking. The feature ranking by consensus is between study 0 and study 1. Ranking is performed according to a score that is the minimum of score 0 and score 1.

As in the prior two-data set example, the data of one study is used for training and the data of the other study is using for testing. Approximately 80% accuracy can be achieved if one trains on the public data and tests on the Stamey data. Only 70% accuracy is obtained in the opposite case. This can be compared to the 90% accuracy obtained when training on one Stamey study and testing on the other in the prior example.

Better “cheating” results are obtained with the consensus features. This serves to validate the consensus features, but the performances cannot be used to predict the accuracy of a classifier on new data.

A SVM is trained using the two best features of study 1 and the samples of study 1 as training data (2003 Stamey data). The data consists of samples of study 0 (public data). A balanced accuracy of 23% is achieved.

Given the differences of distribution between datasets, it is natural that training on one and testing on the other does not yield very high accuracy. The more important question is whether one dataset collected in different conditions can be used to improve performance. For example, when a study is carried out with a new instrument, can old data be re-used to boost the performance?

In all the experiments, “old data” is data that presumably is from a previous study and “new data” is the data of interest. New data is split into a training set and a test set in various proportion to examine the influence of the number of new available samples (in the training data an even proportion is taken of each class). Each experiment is repeated 100 times for random data splits and the balanced success rate is averaged (balanced success rate=average of sensitivity and specificity). When feature selection is preformed, 10 features are selected. All the experiments are performed with the Golub classifier.

There are several ways of re-using “old data”. Features may be selected with the old data only, with the new data only, or a combination of both. Training may be performed with the old data only, with the new data only, or a combination of both. In this last case, a distinction between adapting all the parameters W and b using the “new data” or training W with the “old data” and adapting the bias b only with the “new data” is made.

In this example two sets of experiments, Case 1 and Case 2, were performed.
Case 1: “Old data”=Stamey, “New data”=public
Case 2: “Old data”=public, “New data”=Stamey

The results are different in the two cases, but some trends are common, depending on the amount of new data available.

It helps to use the old data for feature selection and/or training. The combination that does well in both cases is to perform both feature selection and training with the combined old and new data available for training. In case 2, using the new data for feature selection does not improve performance. In fact, performing both feature selection and training with the old data performs similarly in case 2. Training the bias only performs better in case 2 but worse in case 1. Hence, having a stronger influence of the old data helps only in the case when the old data is the public data (perhaps because there is more public data (164 samples, as oppose to only 87 Stamey samples and it is more diverse thus less biased.) The recommendation is to use the old data for feature selection; combine old and new data for training.

Using the “old data” for feature selection and the “new data” for training seems the best compromise in both cases. The recommendation is to use the old data for feature selection and the new data for training

As more “new data” becomes available, using “old data” becomes less necessary and may become harmful at some point. This may be explained by the fact that there is less old data available in case 1. The recommendation is to ignore the old data altogether.

Performing feature selection is a very data hungry operation that is prone to overfitting. Hence, using old data makes sense to help feature selection in the small and medium range of available new data. Because there is less Stamey 2003 data than public data the results are not symmetrical. Without the public data, the classification performances on the public test data are worse using the 10 selected features with Stamey data than without feature selection.

Once the dimensionality is reduced, training can be performed effectively with fewer examples. Hence using old data for training is not necessary and may be harmful when the number of available new data samples exceeds the number of features selected.

When the number of new data samples becomes of the order of the number of old data samples, using old data for training may become harmful.

The publicly available data are very useful because having more data reduces the chances of getting falsely significant genes for gene discovery and helps identifying better genes for classification. The top ten consensus genes are all very relevant to cancer and most of them particularly prostate cancer.

In Example 5, for the problem of tumor vs. normal separation, it was found that a 10-fold cross-validation on the Stamey data (i.e., training on 78 examples) yielded a balanced accuracy of 0.91 with 10 selected features (genes). Using only the publicly available data for selecting 10 genes and training, one gets 0.87 balanced accuracy on Stamey data. Combining the publicly available data and only 20 examples of the Stamey data matches the performance of 0.91 with 10 genes (on average over 100 trials.) If the two datasets as swapped and ten genes are selected and trained on the Stamey 2003 data, then tested on public data, the result is 0.81 balanced accuracy. Incorporating 20 samples of the public data in the training data, a balanced accuracy of 0.89 is obtained on the remainder of the data (on average over 100 trials.)

Normalizing datasets from different sources so that they look the same and can be merged for gene selection and classification is tricky. Using the described normalization scheme, one dataset is used for training and the other for testing, there is a loss of about 10% accuracy compared to training and testing on the same dataset. This could be corrected by calibration. When using a classification system with examples of a “new study”, training with a few samples of the “new study” in addition to the samples of the “old study” is sufficient to match the performances obtained by training with a large number of examples of the “new study” (see results of the classification accuracy item.)

Experimental artifacts may plague studies in which experimental conditions switch between normal and disease patients. Using several studies permits validatation of discoveries. Gene expression is a reliable means of classifying tissues across experimental conditions variations, including differences in sample preparation and microarrays (see results of the classification accuracy item.)

### EXAMPLE 8

### BPH Study

The training set was from Stanford University database from Prof. Stamey; U133A Affymetrix chip, labeled the 2003 dataset in previous example, consisted of the following:

| | |
|---|---|
| Total Number of tissues | 87 |
| BPH | 10 |
| Other | 77 |
| Number of genes | 22283 |

The test set was from Stanford University database from Prof. Stamey; HuGeneFL Affymetrix chip, the “2001 dataset”, and contained the following:

| | |
|---|---|
| Total Number of tissues | 67 |
| BPH | 18 |
| Other | 49 |
| Number of genes | 7129 |

The training data were normalized first by the expression of the reference housekeeping gene ACTB. The resulting matrix was used to compute fold change and average expression magnitude. For computing other statistics and performing machine learning experiments, both the training data and the test data separately underwent the following preprocessing: take the log to equalize the variances; standardize the columns and then the lines twice; take the tanh to squash the resulting values.

The genes were ranked by AUC (area under the ROC curve), as a single gene filter criterion. The corresponding p values (pval) and false discovery rates (FDR) were computed to assess the statistical significance of the findings. In the resulting table, the genes were ranked by p value using training data only. The false discovery rate was limited to 0.01. This resulted in 120 genes. The results are shown in the tables in the compact disk appended hereto containing the BPH results (Appendix 1) and Tumor results (Appendix 2).

The definitions of the statistics used in the ranking are provided in Table 37.

TABLE 37

| Statistic | Description |
|---|---|
| AUC | Area under the ROC curve of individual genes, using training tissues. The ROC curve |
| | (receiver operating characteristic) is a plot of the sensitivity (error rate of the “positive” |
| | class, i.e. the bph tissue error rate) v.s. the specificity (error rate of the “negative” class, |
| | here non-bph tissues. Insignificant genes have an AUC close to 0.5. Genes with an AUC |
| | closer to one are overexpressed in bph. Genes with an AUC closer to zero are |
| | underexpressed. |
| pval | Pvalue of the AUC, used as a test statistic to test the equality of the median of the two |
| | population (bph and non-bph.) The AUC is the Mann-Withney statistic. The test is |
| | equivalent to the Wilcoxon rank sum test. Small pvalues shed doubt on the null |
| | hypothesis of equality of the medians. Hence smaller values are better. To account to the |
| | multiple testing the pvalue may be Bonferroni corrected by multiplying it by the number |
| | of genes 7129. |
| FDR | False discovery rate of the AUC ranking. An estimate of the fraction of insignificant |
| | genes in the genes ranking higher than a given gene. It is equal the pvalue multiplied by |
| | the number of genes 7129 and divided by the rank. |
| Fisher | Fisher statistic characterizing the multiclass discriminative power for the histological |
| | classes (normal, BPH, dysplasia, grade 3, and grade 4.) The Fisher statistic is the ratio of |
| | the between-class variance to the within-class variance. Higher values indicate better |
| | discriminative power. The Fisher statistic can be interpreted as a signal to noise ratio. It |
| | is computed with training data only. |
| Pearson | Pearson correlation coefficient characterizing “disease progression”, with histological |
| | classes coded as 0 = normal, 1 = BPH, 2 = dysplasia, 3 = grade 3, and 4 = grade 4.) A value |
| | close to 1 indicates a good correlation with disease progression. |
| FC | Fold change computed as the ratio of the average bph expression values to the avarage |
| | of the other expression values. It is computed with training data only. A value near one |
| | indicates an insignificant gene. A large value indicates a gene overexpressed in bph; a |
| | small value an underexpressed gene. |
| Mag | Gene magnitude. The average of the largest class expression value (bph or other) |
| | relative to that of the ACTB housekeeping gene. It is computed with training data only. |
| tAUC | AUC of the genes matched by probe and or description in the test set. It is computed |
| | with test data only, hence not all genes have a tAUC. |

The 120 top ranking genes using the AUC criterion, satisfy FDR<=0.01, i.e. including less than 1% insignificant genes. Note that the expression values have undergone the preprocessing described above, including taking the log and standardizing the genes.

An investigation was performed to determine whether the genes are ranked similarly with training and test data. Because training and test data were processed by different arrays, this analysis was restricted to 2346 matched probes. This narrowed down the 120 genes previously selected with the AUC criterion to 23 genes. It was then investigated whether this selection corresponds to genes that also rank high when genes are ranked by the test data. Genes selected are found much faster than by chance. Additionally, 95% of the 23 genes selected with training data are similarly “oriented” (i.e. overexpressed or underexpressed in both datasets.

In some applications, it is important to select genes that not only have discriminative power, but are also salient, i.e. have a large fold change (FC) and a large average expression value of the most expressed category (Mag.) Some of the probes correspond to genes belonging to the same Unigene cluster. This adds confidence to the validity of these genes.

A predictive model is trained to make the separation BPH v.s. non-BPH using the available training data. Its performance is then assessed with the test data (consisting of samples collected at different times, processed independently and with a different microarray technology.) Because the arrays used to process the training and test samples are different, our machine learning analysis utilizes only the 2346 matched probes. To extend the validation to all the genes selected with the training data (including those that are not represented in the test arrays) the set of genes was narrowed down to those having a very low FDR on training data (FDR<=0.01.) In this way, the machine learning analysis indirectly validates all the selected genes.

As previously mentioned, the first step of this analysis was to restrict the gene set by filtering those genes with FDR<=0.01 in the AUC feature ranking obtained with training samples. The resulting 120 genes are narrowed down to 23 by “projecting” them on the 2346 probes common in training and test arrays.

Two feature selection strategies are investigated to further narrow down the gene selection: the univariate and multivariate methods. The univariate method, which consists in ranking genes according to their individual predictive power, is examplified by the AUC ranking. The multivariate method, which consists in selecting subsets of genes that together provide a good predictive power, is examplified by the recursive feature elimination (RFE) method. RFE consists in starting with all the genes and progressively eliminating the genes that are least predictive. (As explained above, we actually start with the set of top ranking AUC genes with FDR<=0.01.) We use RFE with a regularized kernel classifier analogous to a Support Vector Machine (SVM.)

For both methods (univariate and multivariate), the result is nested subsets of genes. Importantly, those genes are selected with training data only.

A predictive model (a classifier) is built by adjusting the model parameters with training data. The number of genes is varied by selecting gene subsets of increasing sizes following the previously obtained nested subset structure. The model is then tested with test data, using the genes matched by probe and description in the test arrays. The hyperparameters are adjusted by cross-validation using training data only. Hence, both feature selection and all the aspect of model training are performed on training data only.

As for feature selection, two different paradigms are followed: univariate and multivariate. The univariate strategy is examplified by the Naive Bayes classifier, which makes independence assumptions between input variables. The multivariate strategy is examplied by the regularized kernel classifier. Although one can use a multivariate feature selection with a univariate classifier and vive versa, to keep things simple, univariate feature selection and classifier methods were used together, and similarly for the multivariate approach.

Using training data only automatically identified 4 outliers which were removed from the rest of the analysis.

Performances were measured with the area under the ROC curve (AUC). The ROC curve plots sensitivity as a function of specificity. The optimal operatic point is application specific. The AUC provides a measure of accuracy independent of the choice of the operating point.

Both univariate and multivariate methods perform well. The error bars on test data are of the order of 0.04, and neither method outperforms the other significantly. There is an indication that the multivariate method (RFE/kernel classifier) might be better for small number of features. This can be explained by the fact that RFE removes feature redundancy. The top 10 genes for the univariate method (AUC criterion) are {Hs.56045, Hs.211933, Hs.101850, Hs.44481, Hs.155597, Hs.1869, Hs.151242, Hs.83429, Hs.245188, Hs.79226,} and those selected by the multivariate method (RFE) are {Hs.44481, Hs.83429, Hs.101850, Hs.2388, Hs.211933, Hs.56045, Hs.81874, Hs.153322, Hs.56145, Hs.83551,}. Note that the AUC-selected genes are different from the top genes in Appendix 1 (BPH results) for 2 reasons: 1) only the genes matched with test array probes are considered (corresponding to genes having a tAUC value in the table) and 2) a few outlier samples were removed and the ranking was redone.

### REFERENCES

The following references are herein incorporated in their entirety.
Alon, et al. (1999) Broad patterns of gene expression revealed by clustering analysis of tumor and normal colon cancer tissues probed by oligonucleotide arrays. PNAS vol. 96 pp. 6745-6750, June 1999, Cell Biology.
Eisen, M. B., et al. (1998) Cluster analysis and display of genome-wide expression patterns Proc. Natl. Acad. Sci. USA, Vol. 95, pp. 14863-14868, December 1998, Genetics.
Alizadeh, A. A., et al. (2000) Distinct types of diffuse large B-cell lymphoma identified by gene expression profiling. Nature, Vol. 403, Issue 3, February, 2000.
Brown, M. P. S., et al. (2000) Knowledge-based analysis of microarray gene expression data by using support vector machines. Proc. Natl. Acad. Sci. USA, Vol. 97, no. 1: 262-267, January, 2000.
Perou, C. M., et al., Distinctive gene expression patterns in human mammar epithelial cells and breast cancers, Proc. Natl. Acad. Sci. USA, Vol. 96, pp. 9212-9217, August 1999, Genetics
Ghina, C., et al., Altered Expression of Heterogeneous Nuclear Ribonucleoproteins and SR Factors in Human, Cancer Research, 58, 5818-5824, Dec. 15, 1998.
Duda, R. O., et al., Pattern classification and scene analysis. Wiley. 1973.
Golub, et al., Molecular Classification of Cancer: Class Discovery and Class Prediction by Gene Expression Monitoring. Science Vol 286, October 1999.
Guyon, I., et al., Structural risk minimization for character recognition. Advances in Neural Information Processing Systems 4 (NIPS 91), pages 471-479, San Mateo Calif., Morgan Kaufmann. 1992.
Guyon, I., et al., Discovering informative patterns and data cleaning. Advances in Knowledge Discovery and Data Mining, pages 181-203. MIT Press. 1996.
Vapnik, V. N., Statistical Learning Theory. Wiley Interscience.1998.
Guyon, I. et al., What size test set gives good error rate estimates? PAMI, 20 (1), pages 52-64, IEEE. 1998.
Boser, B. et al., A training algorithm for optimal margin classifiers. In Fifth Annual Workshop on Computational Learning Theory, pages 144-152, Pittsburgh, ACM. 1992.
Cristianini, N., et al., An introduction to support vector machines. Cambridge University Press.1999.
Kearns, M., et al., An experimental and theoretical comparison of model selection methods. Machine Learning 27: 7-50. 1997.
Shürmann, J., Pattern Classification. Wiley Interscience. 1996.
Mozer, T., et al., Angiostatin binds ATP synthase on the surface of human endothelial cells, PNAS, Vol. 96, Issue 6, 2811-2816, Mar. 16, 1999, Cell Biology.
Oliveira, E. C., Chronic Trypanosoma cruzi infection associated to colon cancer. An experimental study in rats. Resumo di Tese. Revista da Sociedade Brasileira de Medicina Tropical 32(1):81-82, January-February, 1999.
Karakiulakis, G., Increased Type IV Collagen-Degrading Activity in Metastases Originating from Primary Tumors of the Human Colon, Invasion and Metastasis, Vol. 17, No. 3, 158-168, 1997.
Aronson, Remodeling the Mammary Gland at the Termination of Breast Feeding: Role of a New Regulator Protein BRP39, The Beat, University of South Alabama College of Medicine, July, 1999.
Macalma, T., et al., Molecular characterization of human zyxin. Journal of Biological Chemistry. Vol. 271, Issue 49, 31470-31478, December, 1996.
Harlan, D. M., et al., The human myristoylated alanine-rich C kinase substrate (MARCKS) gene (MACS). Analysis of its gene product, promoter, and chromosomal localization. Journal of Biological Chemistry, Vol. 266, Issue 22, 14399-14405, August, 1991.
Thorsteinsdottir, U., et al., The oncoprotein E2A-Pbx1a collaborates with Hoxa9 to acutely transform primary bone marrow cells. Molecular Cell Biology, Vol. 19, Issue 9, 6355-66, September, 1999.
Osaka, M., et al., MSF (MLL septin-like fusion), a fusion partner gene of MLL, in a therapy-related acute myeloid leukemia with a t(11; 17)(q23;q25). Proc Natl Acad Sci USA. Vol. 96, Issue 11, 6428-33, May, 1999.
Walsh, J. H., Epidemiologic Evidence Underscores Role for Folate as Foiler of Colon Cancer. Gastroenterology News. Gastroenterology. 116:3-4, 1999.
Aerts, H., Chitotriosidase—New Biochemical Marker. Gauchers News, March, 1996.
Fodor, S. A., Massively Parallel Genomics. Science. 277:393-395, 1997.
Schölkopf, B., et al., Estimating the Support of a High-Dimensional Distribution, in proceeding of NIPS 1999.
[1] Singh D, et al., Gene expression correlates of clinical prostate cancer behavior Cancer Cell, 2:203-9, Mar. 1, 2002.
[2] Febbo P., et al., Use of expression analysis to predict outcome after radical prostatectomy, The Journal of Urology, Vol. 170, pp. S11-S20, December 2003. Delineation of prognostic biomarkers in prostate cancer. Dhanasekaran S M, Barrette T R, Ghosh D, Shah R, Varambally S, Kurachi K, Pienta K J, Rubin M A, Chinnaiyan A M. Nature. 2001 Aug. 23; 412(6849):822-6.
[3] Comprehensive gene expression analysis of prostate cancer reveals distinct transcriptional programs associated with metastatic disease. LaTulippe E, Satagopan J, Smith A, Scher H, Scardino P, Reuter V, Gerald W L. Cancer Res. 2002 Aug. 1; 62(15):4499-506.
[4] Gene expression analysis of prostate cancers. Luo J H, Yu Y P, Cieply K, Lin F, Deflavia P, Dhir R, Finkelstein S, Michalopoulos G, Becich M. Mol Carcinog. 2002 January; 33(1):25-35
[5] Expression profiling reveals hepsin overexpression in prostate cancer. Magee J A, Araki T, Patil S, Ehrig T, True L, Humphrey P A, Catalona W J, Watson M A, Milbrandt J. Cancer Res. 2001 Aug. 1; 61(15):5692-6.
[6] Analysis of gene expression identifies candidate markers and pharmacological targets in prostate cancer. Welsh J B, Sapinoso L M, Su A I, Kern S G, Wang-Rodriguez J, Moskaluk Calif., Frierson H F Jr, Hampton G M. Cancer Res. 2001 Aug. 15; 61(16):5974-8.
[7] Human prostate cancer and benign prostatic hyperplasia: molecular dissection by gene expression profiling. Luo J, Duggan D J, Chen Y, Sauvageot J, Ewing C M, Bittner M L, Trent J M, Isaacs W B. Cancer Res. 2001 Jun. 15; 61(12):4683-8.
[8] A molecular signature of metastasis in primary solid tumors. Ramaswamy S, Ross K N, Lander E S, Golub T R. Nat. Genet. 2003 January; 33(1):49-54. Epub 2002 Dec. 09.
[9] A compendium of gene expression in normal human tissues. Hsiao L L, Dangond F, Yoshida T, Hong R, Jensen R V, Misra J, Dillon W, Lee K F, Clark K E, Haverty P, Weng Z, Mutter G L, Frosch M P, Macdonald M E, Milford E L, Crum C P, Bueno R, Pratt R E, Mahadevappa M, Warrington J A, Stephanopoulos G, Stephanopoulos G, Gullans S R. Physiol Genomics. 2001 Dec. 21; 7(2):97-104.
[10] Molecular classification of human carcinomas by use of gene expression signatures. Su A l, Welsh J B, Sapinoso L M, Kern S G, Dimitrov P, Lapp H, Schultz P G, Powell S M, Moskaluk C A, Frierson H F Jr, Hampton G M. Cancer Res. 2001 Oct. 15; 61(20):7388-93.
[11] Gene expression analysis of prostate cancers. Jian-Hua Luo*, Yan Ping Yu, Kathleen Cieply, Fan Lin, Petrina Deflavia, Rajiv Dhir, Sydney Finkelstein, George Michalopoulos, Michael Becich.
[12] Transcriptional Programs Activated by Exposure of Human Prostate Cancer Cells to Androgen”, Samuel E. DePrimo, Maximilian Diehn, Joel B. Nelson, Robert E. Reiter, John Matese, Mike Fero, Robert Tibshirani, Patrick O. Brown, James D. Brooks. Genome Biology, 3(7) 2002
[13] A statistical method for identifying differential gene-gene co-expression patterns, Yinglei Lai, Baolin Wu, Liang Chen and Hongyu Zhao. Bioinformatics vol. 20 issue 17.
[14] Induction of the Cdk inhibitor p21 by LY83583 inhibits tumor cell proliferation in a p53-independent manner Dimitri Lodygin, Antje Menssen, and Heiko Hermeking, J. Clin. Invest. 110:1717-1727 (2002).
[15] Classification between normal and tumor tissues based on the pair-wise gene expression ratio. YeeLeng Yap, XueWu Zhang, M T Ling, XiangHong Wang, Y C Wong, and Antoine Danchin BMC Cancer. 2004; 4: 72.
[16] Kishino H, Waddell P J. Correspondence analysis of genes and tissue types and finding genetic links from microarray data. Genome Inform Ser Workshop Genome Inform 2000; 11: 83-95.
[17] Proteomic analysis of cancer-cell mitochondria. Mukesh Verma, Jacob Kagan, David Sidransky & Sudhir Srivastava, Nature Reviews Cancer 3, 789-795 (2003);
[18] Changes in collagen metabolism in prostate cancer: a host response that may alter progression. Burns-Cox N, Avery N C, Gingell J C, Bailey A J. J. Urol. 2001 November; 166(5): 1698-701.
[19] Differentiation of Human Prostate Cancer PC-3 Cells Induced by Inhibitors of Inosine 5′-Monophosphate Dehydrogenase. Daniel Florykl, Sandra L. Tollaksen2, Carol S. Giometti2 and Eliezer Hubermanl Cancer Research 64, 9049-9056, Dec. 15, 2004.
[20] Epithelial Na, K-ATPase expression is down-regulated in canine prostate cancer; a possible consequence of metabolic transformation in the process of prostate malignancy Ali Mobasheri, Richard Fox, lain Evans, Fay Cullingham, Pablo Martin-Vasallo and Christopher S Foster Cancer Cell International 2003, 3:8 Stamey, T. A., McNeal, J. E., Yemoto, C. M., Sigal, B. M, Johnstone, I. M. Biological determinants of cancer progression in men with prostate cancer. J. Amer. Med. Assoc., 281: 1395-4000, 1999.
Stamey, T. A., Warrington, J. A., Calwell, M. C., Chen, Z., Fan, Z., Mahadevappa, M. et al: Molecular genetic profiling of Gleason grade 4/5 cancers compared to benign prostate hyperplasia. J. Urol, 166:2171, 2001.
Stamey, T. A., Caldwell, M. C., Fan, Z., Zhang, Z., McNeal, J. E., Nolley, R. et al: Genetic profiling of Gleason grade 4/5 prostate cancer: which is the best prostatic control? J Urol, 170:2263, 2003.
Chen, Z., Fan, Z., McNeal, J. E., Nolley, R., Caldwell, M., Mahavappa, M., et al: Hepsin and mapsin are inversely expressed in laser capture microdissectioned prostate cancer. J Urol, 169:1316, 2003.
McNeal, J E: Prostate. In: Histology for Pathologists 2nd ed. Edited by Steven S. Sternberg, Philadelphia: Lippincott-Raven Publishers, chapt. 42, pp. 997-1017, 1997.
Phillip G. Febbo and William R. Sellers. Use of expression analysis to predict outcome after radical prostatectomy. The journal of urology, vol 170, pp 811-820, December 2003.
Phillip G. Febbo and William R. Sellers. Use of expression analysis to predict outcome after radical prostatectomy. The journal of urology, vol 170, pp 811-820, December 2003.
Stamey, T. A., Caldwell, M. C., Fan, Z., Zhang, Z., McNeal, J. E., Nolley, R., et al: Genetic profiling of Gleason grade 4/5 prostate cancer: which is the best prostatic control? J Uroll 170:2263, 2003.
Stamey, T. A., Caldwell, M. C., et al. Morphological, Clinical, and Genetic Profiling of Gleason Grade 4/5 Prostate Cancer. Unpublished technical report. Stanford University, 2004.
Chen, Z., Fan, Z., McNeal, J. E., Nolley, R., Caldwell, M. Mahadevappa, M., et al: Hepsin and Mapsin are inversely expressed in laser capture microdissected prostate cancer. J Urol, 169:1316, 2003.
Tibshirani, Hastie, Narasimhan and Chu (2002) Diagnosis of multiple cancer types by shrunken centroids of gene expression, PNAS 2002 99:6567-6572 (May 14).
Welsh, J. B., Sapinoso, L. M., Su, A. I., Kern, S. G., Wnag-Rodriguez, J., Moskaluk, C. A., et al: Analysis of gene expression identifies candidate markers and pharmacological targets in prostate cancer. Cancer Res. 61:5974, 2001. other available data Masanori Nogushi, Thomas A. Stamey, John E. McNeal, and Cheryl E. M. Yemoto, An analysis of 148 consecutive transition zone cancers: clinical and histological characteristics. The journal of urology, vol. 163, 1751-1755, June 2000.
G. Kramer, G. E. Steiner, P. Sokol, R. Mallone, G. Amann and M. Marberger, Loss of CD38 correlates with simultaneous up-regulation of human leukocyte antigen-DR in benign prostatic glands, but not in fetal or androgen-ablated glands, and is strongly related to gland atrophy. BJU International (March 2003), 91.4.
Beer T M, Evans A J, Hough K M, Lowe B A, McWilliams J E, Henner W D. Polymorphisms of GSTP1 and related genes and prostate cancer risk. Prostate Cancer Prostatic Dis. 2002; 5(1):22-7.
Jacques Lapointe, et al. Gene expression profiling identifies clinically relevant subtypes of prostate cancer. Proc Natl Acad Sci USA. 2004 Jan. 20; 101 (3): 811-816.
Caine G J, Blann A D, Stonelake P S, Ryan P, Lip G Y. Plasma angiopoietin-1, angiopoietin-2 and Tie-2 in breast and prostate cancer: a comparison with VEGF and Flt-1. Eur J Clin Invest. 2003 October; 33(10):883-90.
Y Tokugawa, I Kunishige, Y Kubota, K Shimoya, T Nobunaga, T Kimura, F Saji, Y Murata, N Eguchi, H Oda, Y Urade and O Hayaishi, Lipocalin-type prostaglandin D synthase in human male reproductive organs and seminal plasma. Biology of Reproduction, Vol 58, 600-607, 1998
Mukhtar H, Lee I P, Bend J R. Glutathione S-transferase activities in rat and mouse sperm and human semen. Biochem Biophys Res Commun. 1978 Aug. 14; 83(3): 1093-8.

1. A biomarker for screening, predicting, and monitoring prostate cancer volume comprising any combination of the genes identified by Unigene ID numbers of the table in FIG. 19.

2. A biomarker for screening, predicting, and monitoring prostate cancer comprising two or more genes selected from the group consisting of cDNA DKFZp564A072, GSTP1, HPN, TACSTD1, ANGPT1, PTGDS, RRAS, Ncoa4, Pak6-ESTs, Tmf1-ESTs (ARA160), 2010301M18Rik (Cyp2c19), Acpp, Adh1, Akr1b3 (aldose reductase), Aldh1a1 (ALDH1), Dhcr24 (seladin-1), Folh1 (PSMA), Gpx5, Klk4, Morf-pending, Myst1, Ngfa, Ppap2a, Ppap2b, Srd5a2, Tgm4 (hTGP), Tmprss2, Anxa7, Apoe, Cdhl, Enh-pending (Lim), Gstpl, Hpn (Hepsin), Olfr78 (PSGR), Pov1, Psca, Pten, Ptov1, Sparcl1 (HEVIN), Steap, Tnfrsf6 (FAS), C20orf1-Rik (FLS353), Fat, Fbxl11, Igf1, Igfbp5, Kcnmb1, Mta1, Mybl2, Oxr1 (C7), Ppap2b, Rab5a, Rap1a, and Sfrp4.

3. A method for distinguishing between benign prostate hyperplasia (BPH) and tumor in prostate tissue comprising screening for gene expression of ten or fewer genes selected from the group of genes identified by Unigene ID numbers of the table in FIG. 10, FIG. 24, FIG. 25 and Table 38.

4. The method of claim 3, wherein the gene expression is tested in serum.

5. The method of claim 3, wherein the gene expression is tested in biopsied prostate tissue.

6. The method of claim 3, wherein the gene expression is tested in semen.

7. A method for distinguishing between benign prostate hyperplasia (BPH) and tumor in prostate tissue comprising screening for gene expression of more than ten genes selected from the group of genes identified by Unigene ID numbers of FIG. 10, FIG. 24, FIG. 25 and Table 38.

8. The method of claim 7, wherein the gene expression is tested in serum.

9. The method of claim 7, wherein the gene expression is tested in biopsied prostate tissue.

10. The method of claim 7, wherein the gene expression is tested in semen.

11. A method for distinguishing between G3 and G4 prostate cancer tumors and non G3 and G4 tissue comprising screening for gene expression of 100 or fewer genes selected from the group of genes identified by Unigene ID numbers of the tables of FIG. 11, FIG. 20, FIG. 23, FIG. 26, FIG. 27 or Table 38.

12. The method of claim 11, wherein the gene expression is tested in serum.

13. The method of claim 11, wherein the gene expression is tested in biopsied prostate tissue.

14. The method of claim 11, wherein the gene expression is tested in semen.

15. A method for distinguishing between G3 and G4 prostate cancer tumors and non G3 and G4 tissue comprising screening for gene expression of 100 or more genes selected from the group of genes identified by Unigene ID numbers of the tables in FIG. 1, FIG. 20, FIG. 23, FIG. 26, FIG. 27 or Appendix 2.

16. The method of claim 15, wherein the gene expression is tested in serum.

17. The method of claim 15, wherein the gene expression is tested in biopsied prostate tissue.

18. The method of claim 15, wherein the gene expression is tested in semen.

19. A method for distinguishing between G4 prostate cancer tumors and non G4 tissue comprising screening for gene expression of 100 or fewer genes selected from the group of genes identified by Unigene ID numbers of the table of FIG. 12.

20. The method of claim 19, wherein the gene expression is tested in serum.

21. The method of claim 19, wherein the gene expression is tested in biopsied prostate tissue.

22. The method of claim 19, wherein the gene expression is tested in semen.
